(19) United States
(12) Patent Application Publication
(10) Pub. No.: US 2026/0256690 A1
(43) Pub. Date: Sep. 3, 2026

(54) COMPOSITIONS AND METHODS FOR IN UTERO DELIVERY

(71) Applicant: Yale University, New Haven, CT (US)

(72) Inventors: W. Mark Saltzman, New Haven, CT (US); Peter M. Glazer, Guilford, CT (US); Adele S. Ricciardi, New Haven, CT (US); David H. Stitelman, Branford, CT (US); James Stephen Farrelly, Milford, CT (US); Anthony Bianchi, Liverpool, NY (US); Alexandra S. Piotrowski-Daspit, Wetherfield, CT (US); Amy Kauffman, Cary, NY (US)

(21) Appl. No.: 19/029,285

(22) Filed: Jan. 17, 2025

Related U.S. Application Data
(63) Continuation of application No. 18/397,091, filed on Dec. 27, 2023, now Pat. No. 12,268,774.
Application No. 18/397,091 is a continuation of application No. 16/603,152, filed on Apr. 4, 2018, now abandoned.
(60) Provisional application No. 62/481,562, filed on Apr. 4, 2017.
(60) Provisional application No. 62/489,377, filed on Apr. 24, 2017.
(60) Provisional application No. 62/589,275, filed on Nov. 21, 2017.

(51) Int. Cl. A61K 9/00; A61K 9/16; A61K 9/51; A61K 38/18; A61K 47/60; A61K 48/00; A61P 7/00; A61P 11/00

(52) U.S. Cl. CPC A61K 9/0034; A61K 9/0019; A61K 9/1647; A61K 9/1652; A61K 9/5153; A61K 38/1825; A61K 47/60; A61K 48/0041; A61P 7/00; A61P 11/00

(57) ABSTRACT

Compositions and methods for fetal or in utero delivery of active agents are provided. The compositions are most typically administered intravenously via the vitelline vein, umbilical vein, or directly into the amniotic cavity of a pregnant mother. Fibroblast growth factor is to correct structural defects of neural tissue.

### CROSS-REFERENCE TO RELATED APPLICATIONS

This application is a continuation of U.S. Ser. No. 18/397,091 filed Dec. 27, 2023, which is a continuation of U.S. Ser. No. 16/603,152 filed Oct. 4, 2019, now abandoned, which is a National Phase application under 35 U.S.C. § 371 of PCT/US2018/026116, filed Apr. 4, 2018, which claims the benefit of and priority to U.S. Ser. No. 62/481,562, filed Apr. 4, 2017, U.S. Ser. No. 62/489,377, filed Apr. 24, 2017, and U.S. Ser. No. 62/589,275, filed Nov. 21, 2017, each of which is specifically incorporated by reference herein in its entirety.

### STATEMENT REGARDING FEDERALLY SPONSORED RESEARCH

This invention was made with government support under AI112443 and HL125892 awarded by the National Institutes of Health. The government has certain rights in the invention.

### FIELD OF THE INVENTION

The field of the invention is generally related to compositions and methods for in utero delivery of active agents, particularly those in which the active agent is encapsulated within, surrounded by, and/or dispersed in polymeric microparticles or nanoparticles.

### BACKGROUND OF THE INVENTION

Every year an estimated 8 million children are born worldwide with severe structural birth defects or genetic disorders. Structural birth defects are abnormalities in the structure of body parts such as cleft palate, heart defects, club foot, missing and abnormal limbs. Most structural defects develop in the earliest weeks of pregnancy when all of the organs and the skeleton are forming. Myelomeningocele (MMC), an open form of spina bifida, is both the most common and most severe form of neural tube defect associated with long-term survival. MMC occurs in up to 3000 live births in the United States each year, and this does not include the estimated 63% of MMC pregnancies in which the fetus terminated, either spontaneously or intentionally. Patients with MMC have a boney spinal defect but no effective skin or soft tissue covering to protect the spinal cord and meninges which herniate posteriorly through the defect. Despite improved multi-disciplinary care, the neonatal mortality rate for MMC patients remains as high as 10%, and only approximately half of those who survive will be able to function independently as adults. Fetal correction of MMC results in better outcomes, and some have proposed that stem cells at the MMC skin edges can be recruited with the aid of growth factors to close the MMC. Experiments with gelatin sponges impregnated with fibroblast growth factor (FGF) have been investigated in rat and sheep models of MMC.

Patients with the mildest type of neural tube defect, spina bifida occulta, have an open defect in the boney vertebral arches that normally protect the underlying spinal cord. Despite having this boney defect, these patients are usually neurologically normal and have no signs or symptoms except for a small dimple or tuft of hair overlying the defect.

Hemoglobinopathies are the most commonly inherited single-gene disorders, with a global carrier frequency of over 5%. Depending on the severity of the disease, children affected by β-thalassemia may require lifelong transfusions or bone marrow transplantation, which can lead to serious complications such as iron overload, sepsis, or graft-versus-host disease. Recent advances in non-invasive genetic testing allow for early gestation diagnosis of genetic disorders such as thalassemia, providing a window during which genetic correction could be achieved prior to birth.

In utero gene therapy thus far has focused on stem cell transplantation and viral-mediated gene delivery, approaches that do not allow for correction of a gene in its endogenous environment. However, at least one report describes the in utero delivery of oligonucleotides for the treatment of Duchenne Muscular Dystrophy (Cai et al., “In Utero Delivery of Oligodeoxynucleotides for Gene Correction”, Chapter 26, Francesca Storici (ed.), Gene Correction: Methods and Protocols, Methods in Molecular Biology, 1114 (2014)). Site-specific gene editing to correct disease-causing mutations can be administered to postnatal animals via the intravenous or inhalational administration of polymeric, biodegradable nanoparticles loaded with peptide nucleic acids (PNAs) and single-stranded donor DNAs. In a recent report, chitosan-DNA nanoparticles were used for in utero gene therapy (Yang et al. Journal of Surgical Research, 171:691-699 (2011)).

Improved compositions and methods for in utero therapeutics and diagnostics are needed, where, for example, diseases or disorders can be addressed prior to the point where irreparable harm is done to tissues and/or organ systems of the embryo or fetus.

Therefore, it is an object of the invention to provide compositions and methods for in utero delivery.

### SUMMARY OF THE INVENTION

Compositions and methods for in utero delivery of active agents allow therapeutic and diagnostic molecules to be delivered to an embryo or fetus in need thereof. In some embodiments, the methods deliver an effective amount of a composition to the cells of the embryo or fetus, without delivering an effective amount of the composition to the mother of the embryo or fetus.

The therapeutic or diagnostic active agent can be encapsulated, entrapped or complexed to biocompatible particles such as nano- or microparticles. The particles can be formulated for cellular internalization or to remain predominately extracellular, releasing their cargo in a paracrine-like fashion. In some embodiments, the particles do not cross the placenta. These nano- and microparticles can be engineered to attach to specific targets or to release their chemical payload at different rates. The active agent can also be administered to a subject in utero without particles.

Active agents for in utero delivery include, but are not limited to therapeutic, nutritional, diagnostic, or prophylactic agents, and gene editing compositions. The active agents can be small molecule active agents or biomacromolecules, such as proteins, polypeptides, sugars or polysaccharides, liporproteins or nucleic acids. Suitable small molecule active agents include organic and organometallic compounds. The small molecule active agents can be a hydrophilic, hydrophobic, or amphiphilic compound. The nucleic acids can be oligonucleotide drugs such as DNA, RNAs, antisense, aptamers, small interfering RNAs, ribozymes, external guide sequences for ribonuclease P, and triplex forming agents. In some embodiments, the methods are utilized to deliver active agents such as small molecules, peptides, nucleic acids, peptide nucleic acids (PNAs), locked nucleic acids (LNAs), morpholino oligomers (PMOs), etc., absent a gene editing composition. In some embodiments, the only active agent is a gene editing composition. Exemplary diagnostic agents include paramagnetic molecules, fluorescent compounds, magnetic molecules, and radionuclides, x-ray imaging agents, and contrast agents.

The methods typically include administering to an embryo or fetus, or the pregnant mother thereof, an effective amount of an active agent, optionally encapsulated or entrapped in or otherwise associated with particles. Gene editing methods typically include administering to an embryo or fetus, or the pregnant mother thereof, an effective amount a gene editing composition optionally encapsulated or entrapped in particles, alone or in combination with additional active agents. In some methods, active agent or particles including active agent are delivered in utero by injecting and/or infusing the particles into a vein or artery, such as such the vitelline vein or the umbilical vein, or into the amniotic sac of an embryo or fetus.

Particle compositions for extracellular and intracellular delivery of active agents include, but are not limited to, gene editing compositions are also provided and particularly advantageous for use with in utero applications. The particle are preferably made of biodegradable polymers such as polymers or copolymers of lactic acid, glycolic acid, degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), poly(amine-co-ester) polymers, or a combination of any two or more of the foregoing. The particles can be or include poly(lactic-co-glycolic acid) PLGA. In some embodiments, the particles include poly(lactic-co-glycolic acid) (PLGA), poly(beta-amino) ester (PBAE), or a combination thereof. The particles can be formed of or contain one or more poly(amine-co-ester), poly(amine-co-amide), poly(amine-co-ester-co-ortho ester) or a combination thereof. Particles can include or be formed of alginate, chitosan, poly(HEMA) or other acrylate polymers and copolymers. Particles can further include a targeting moiety, a cell penetrating peptide, or a combination thereof. The particles used in the compositions can be of single species or a mixture of two or more different species of particles.

The particles can include an active agent, such as a gene editing composition, suitable for treatment of and can be administered in an effective amount to treat a disease or disorder.

In some embodiments, the formulations and methods provided for the controlled local release of growth factors to cause skin or soft tissue to grow over the exposed spinal cord of the fetal or embryonic subject. The formulation can include biocompatible particles which preferentially binds to MMC defects in utero and effectively release therapeutic agents to induce at least partial skin or soft tissue coverage of the defect. The formulation can be delivered in a minimally invasive fashion through an intra-amniotic injection.

Exemplary diseases and disorders which can be treated include, but are not limited to, genetic disorders such as hemophilia, hemoglobinopathies, cystic fibrosis, xeroderma pigmentosum, muscular dystrophy, Type 2 diabetes, diseases of the liver such as Wilson's disease and hemochromatosis, or diseases of the central nervous system including Fredrich's ataxia, Huntington's disease, spinal muscular atrophy, tuberous sclerosis and lysosomal storage diseases. Gene editing compositions include, for example, triplex forming molecules, pseudocomplementary oligonucleotides, a CRISPR system, zinc finger nucleases (ZFN), transcription activator-like effector nucleases (TALEN), small fragment homologous replacement, and intron encoded meganucleases. A particularly preferred gene editing composition is triplex-forming peptide nucleic acids (PNAs) having one or more substitutions at the γ position of the backbone for increased DNA binding affinity.

Gene editing composition can include or be used in conjunction with or combination with a donor oligonucleotide. The donor oligonucleotide can facilitate genome modification. For example, the donor oligonucleotide can include a sequence corresponding to the target sequence or to the target gene. In some embodiments, the gene editing composition facilitates insertion of the donor oligonucleotide within the target sequence. The donor oligonucleotide can include a wildtype sequence of the target sequence. The donor oligonucleotide can include a corrective sequence. The insertion of the corrective sequence can result in a wildtype sequence at the target sequence or a target gene.

A gene editing composition typically modifies a target sequence within a genome. The gene editing composition can, for example, modify a target sequence within a genome by reducing or preventing expression of the target sequence. In some embodiments, the gene editing composition induces single-stranded or double-stranded breaks in the target sequence. In some embodiments, the gene editing composition induces formation of a triplex within the target sequence.

The target sequence can be, for example, within a fetal genome. In some embodiments, the gene editing composition does not modify a target sequence within a maternal genome. The target sequence can be present in the fetal genome and the maternal genome and the sequences can be identical. In some embodiments, the target sequence in the fetal genome and maternal genome are not identical, or the target sequence is completely absent from the maternal genome. The fetal genome and the maternal genome can be isolated, derived, or obtained from genetically-related individuals or from genetically un-related individuals. For example, the mother carrying the embryo or fetus can be a surrogate. In some embodiments, the fetus and the mother have the same disease or disorder or are at risk for developing the same disease or disorder. In other embodiments, the fetus has a disease or disorder or is at risk for developing a disease or disorder, and the mother neither has the disease or disorder nor has any risk of developing a disease or disorder. In some embodiments, the composition enters only fetal bodily fluids or tissues, and does not enter maternal bodily fluids or tissues. In some embodiments, the composition is administered to a fetus or to the mother once or more when the fetus is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, and/or 36 weeks of age.

In some embodiments, the fetal genome includes one or more mutations in a coding sequence or a non-coding sequence corresponding to a target gene that either indicates the fetus is at risk of developing a disease or disorder or that indicates that the fetus has a disease or disorder. The mutation can include, for example, a substitution, an insertion, a deletion, an indel, an inversion, a frameshift, or a transposition. The mutation can cause, for example, a transcriptional or translational truncation, altered transcriptional splicing, early termination of transcription or translation, variant transcriptional regulation or variant epigenetic regulation.

In some embodiments, a coding sequence or a non-coding sequence corresponding to the target gene includes the target sequence. A coding sequence corresponding to the target gene can include one or more exon(s) encoding a product of the target gene. A non-coding sequence corresponding to the target gene can include one or more transcriptional regulator(s), enhancer(s), superenhancer(s), intron(s), and/or regulatory RNAs that selectively bind a transcript of the target gene. In some embodiments, the one or more transcriptional regulator(s) includes a sequence encoding a promoter. In some embodiments, one or more regulatory RNAs that selectively bind a transcript of the target gene include one or more miRNA(s).

DNA-binding triplex forming molecules are also provided. The triplex forming molecules can be utilized in all manners of gene modification including those methods both with and without a potentiating agent. The triplex forming composition (also referred to herein as a triplex-forming molecule) typically includes a Hoogsteen binding peptide nucleic acid (PNA) segment and a Watson-Crick binding PNA segment, which in embodiments collectively totals no more than about 25, 50, 75, or 100 nucleobases, wherein the two segments can bind or hybridize to a target region having a polypurine stretch in a cell's genome to induce strand invasion, displacement, and formation of a triple-stranded molecule among the two PNA segments and the polypurine stretch. The Hoogsteen binding segment binds to the target duplex by Hoogsteen binding for a length of at least five nucleobases, and the Watson-Crick binding segment typically binds to the target duplex by Watson-Crick binding for a length of least five nucleobases. In some embodiments, the triplex forming molecule is a tail-clamp PNA oligomer.

In preferred embodiments, one or more of the PNA residue(s) (also referred to herein as ‘residue(s)’) of a PNA oligomer include at least one side chain modification at the gamma position of the backbone. The side chain modification at the γ position of the γPNA residue(s) of a PNA oligomer can be, for example, the side chain of an amino acid selected from the group consisting of alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. In some embodiments, the side chain modification at the γ position of the PNA residue or residues is a diethylene glycol moiety (“miniPEG”). In some embodiments, all of the peptide nucleic acid residues in the Hoogsteen-binding portion only, all of the peptide nucleic acid residues in the Watson-Crick-binding portion only, or all of the peptide nucleic residues in the PNA oligomer have at least one gamma modification of a backbone carbon. In some embodiments, alternating residues in the Hoogsteen-binding portion only, the Watson-Crick-binding portion only, or across the entire PNA oligomer have at least one gamma modification of a backbone carbon. Specific exemplary sequences are provided below.

In some embodiments, one or more of the cytosine nucleobases of the PNA oligomer is replaced with a clamp-G (9-(2-guanidinoethoxy) phenoxazine). In some embodiments, the Hoogsteen binding segment of the PNA oligomer has one or more chemically modified cytosine nucleobases selected from the group consisting of pseudocytosine, pseudoisocytosine, and 5-methylcytosine. The Watson-Crick binding segment preferably includes a nucleobase sequence of up to fifteen nucleobases that binds to the target duplex by Watson-Crick binding outside of the triplex. In preferred embodiments, the two segments are linked by a linker, for example, between 1 and 10 units of 8-amino-3,6-dioxaoctanoic acid.

Gene editing compositions such as PNA oligomers can include a targeting moiety, a cell penetrating peptide, or a combination thereof.

The methods can include administration of a gene modification potentiating agent. Exemplary potentiating agents include, but are not limited to, SCF, a CHK1 or ATR inhibitor, a DNA polymerase alpha inhibitor, a heat shock protein 90 inhibitor (HSP90i) or a combination of any two or more of the foregoing.

### BRIEF DESCRIPTION OF THE DRAWINGS

FIG. 1A is a schematic showing a strategy for targeted correction of a β-globin gene IVS2-654 (C->T) mutation in β-globin/GFP transgenic mice using triplex-forming tail clamp PNAs (tcPNAs) and donor DNAs. FIG. 1B is an illustration showing tcPNA and γtcPNA oligomers designed to bind to the homopurine regions within intron 2 of the human β-globin gene in the vicinity of the thalassemia-associated mutation IVS2-654 (C->T), and a scrambled control sequence. FIG. 1C is an illustration of the chemical structures of DNA (and RNA), unmodified (“classic”) PNA and (right-handed “R”) miniPEG gamma PNA (^{MP}γPNA) units. FIG. 1D is a bar graph showing gene correction of the IVS2-654 (C->T) mutation within the β-globin/GFP fusion gene in mouse bone marrow cells treated ex vivo with blank NPs and NPs containing donor DNA alone or in combination with tcPNA3. The % GFP+ cells among mouse bone marrow cells was determined by flow cytometry and indicates successful gene editing. Data are shown as mean±s.e., n=3; statistical analysis was performed with student's t-test, asterisk, p<0.05. FIG. 1E is a line graph showing release of total nucleic acids (PNAs in combination with donor DNA: γtcPNA4, tcPNA1, tcPNA2, tcPNA3 or γtcPNA4-Scr; or DNA donor alone) from PLGA nanoparticles during incubation at 37° C. in PBS. At 64 hrs, the residual nucleic acid in the NP pellet was extracted and the total nucleic acid load was calculated as a sum of absorbance obtained from the pellet and supernatant. FIG. 1F is a bar graph showing % GFP+ cells determined by flow cytometry among mouse bone marrow cells (from β-globin/GFP transgenic mice) after ex vivo treatment with PLGA NPs containing tcPNA1, γtcPNA4, or γtcPNA4-Scr plus donor DNAs. Replicates and statistics as above for FIG. 1D. FIG. 1G is a bar graph showing mouse total bone marrow cells were treated with either blank NPs or NPs containing γtcPNA4 and donor DNA and were plated for a colony-forming cell assay in methylcellulose medium with selected cytokines for growth of granulocyte/macrophage colonies (CFU-G, CFU-M and CFU-GM) or combined colonies (CFU-GEMM, granulocyte, erythroid, monocyte/macrophage, megakaryocyte. Numbers of each type of colony per 300,000 plated cells are shown. Data are shown as mean±s.d., n=3. FIG. 1H is a bar graph showing the results of a comet assay to measure DNA breaks in NP-treated bone marrow cells. Cells were treated with NPs containing either tcPNA1/donor DNA, γtcPNA4/donor DNA, or bleomycin/donor DNA, as indicated. DNA tail moment provides a measurement of the extent of breaks. Data are shown as mean s.e., n=3.

FIG. 2A is a bar graph showing % GFP expression in treated mouse bone marrow cells based on selected hematopoietic cell surface markers. Total bone marrow was treated with NPs containing either tcPNA1/donor DNA or γtcPNA4/donor DNA, and then the cells were stained using antibodies specific for the indicated markers and assayed by flow cytometry for marker and GFP expression. Data are shown as mean±s.e., n=3; statistical analysis was performed with student's t-test, asterisk, p<0.05. FIG. 2B is a bar graph showing % GFP expressing CD117 (c-Kit+) cells after ex vivo treatment with NPs carrying γtcPNAs and donor DNAs versus with blank NPs. Data are shown as mean±s.e., n=3; statistical analysis was performed with student's t-test, asterisk, p<0.05. FIG. 2C is a bar graph showing % GFP expressing CD117+ cells from β-globin/GFP transgenic mice after ex vivo treatment with NPs containing γtcPNA4/donor DNA with or without prior treatment with the c-Kit ligand, SCF. Data are shown as mean±s.e., n=3; statistical analysis was performed with student's t-test, asterisk, p<0.05. FIG. 2D is a bar graph showing % GFP expressing CD117+ cells isolated from β-globin/GFP transgenic mice after ex vivo treatment with NPs containing γtcPNA4/donor DNA in the presence or absence of selected c-Kit pathway kinase inhibitors: dasatinib (inhibits c-Kit), MEK162 (inhibits mitogen/extracellular signal-regulated kinase, MEK) and BKM120 (inhibits phosphatidylinositol-3-kinase, PI3K). Data are shown as mean±s.e., n=3; statistical analysis was performed with student's t-test, asterisk, p<0.05. FIGS. 2E and 2F are bar graphs showing qPCR determination of mRNA expression levels of BRCA2 (2E) and Rad51 (2F) in CD117− and CD117+ cells. FIG. 2G is a heat map showing up-regulated genes involved in DNA repair pathways in CD117+ cells with or without treatment with SCF; rows are clustered by Euclidean distance measure. FIG. 2H is a bar graph showing the results of a gene assay for homology-dependent repair (HDR) activity in the presence or absence of selected c-Kit pathway kinase inhibitors: dasatinib (inhibits c-Kit), MEK162 (inhibits mitogen/extracellular signal-regulated kinase, MEK) and BKM120 (inhibits phosphatidylinositol-3-kinase, PI3K). Inset shows a diagram of the luciferase reporter gene assay for repair of a nuclease-induced double-strand break by homology-dependent repair (HDR). Luciferase expression occurs only after homologous recombination and is scored as % reactivation of the DSB-damaged plasmid, normalized to a transfection control. FIG. 2I is a bar graph showing the results of an HDR assay in CD117+ cells with or without the addition of SCF. Data are shown as mean±s.e., n=3; statistical analysis was performed with student's t-test, asterisk, p<0.05. FIG. 2J is a bar graph showing the results of an HDR assay in DLD-1 cells either proficient or deficient in the homology dependent repair factor BRCA2 as a validation of the assay. Data are shown as mean±s.e., n=3; statistical analysis was performed with student's t-test, asterisk, p<0.05.

FIGS. 3A and 3B are dot plots showing frequencies of gene editing (GFP expression) in bone marrow (3A) and spleen (3B) cells from β-globin/GFP transgenic mice (6 mice per group) injected or not (as indicated) with 15.6 μg of SCF i.p. followed by a single treatment of 4 mg of NPs injected intravenously. Each group received either blank NPs or NPs containing γtcPNA4 and donor DNA, with or without SCF and were harvested and analyzed two days later. Each data point represents analysis of cells from a single mouse. Statistical analyses were performed using student's t-test: asterisk, p<0.05. FIG. 3C is a bar graph showing the results of deep-sequencing analysis to quantify the frequency of targeted gene editing (% modification frequency IVS2-654 (T->C)) in vivo in CD117+ cells from bone marrow and spleen of β-globin/GFP mice treated as described for FIGS. 3A and 3B. Error bars indicate standard error of proportions.

FIGS. 4A-4C are line graphs showing blood hemoglobin levels (g/dl) of thalassemic mice treated with blank NPs (4A), SCF plus scrambled gfpγtcPNA4-Scr/donor DNA (4B) NPs, or with SCF plus γtcPNA4/donor DNA (4C) NPs performed at the indicated times after treatment. Each line represents an individual mouse followed over time. FIG. 4D is a bar graph showing reticulocyte counts (% of total RBCs) calculated in blood smears from thalassemic mice treated with either blank NPs or with NPs containing γtcPNA4/donor DNA plus SCF on days 0 and 36 post treatment. FIG. 4E is a bar graphs showing the % gene modification (T->C) as determined by deep-sequencing analysis of genomic DNA from bone marrow cells after treatment of thalassemic mice with either blank NPs or with NPs containing and γtcPNA4/donor DNA plus SCF.

FIG. 5A is a flow diagram illustrating a GFP/beta globin gene correction assay. FIG. 5B is a bar graph showing gene correction of cells treated with nanoparticles containing tcPNA1 and donor DNA alone, or in combination with an ataxia telangiectasia and Rad3-related protein (ATR) pathway inhibitor (MIRIN, KU5593, VE-821, NU7441, LCA, or L189). FIG. 5C is a bar graph showing gene correction of cells treated with nanoparticles containing tcPNA1 and donor DNA alone, or in combination with a Checkpoint Kinase 1 inhibitor (Chk1i) (SB218075), a DNA polymerase alpha inhibitor (Aphi) (aphidicolin) or a polyADP ribose polymerase (PARPi) (AZD-2281 (olaparib)). FIG. 5D is a bar graph showing gene correction of control (blank), and cells treated with nanoparticles containing tcPNA1 and donor DNA alone, or in combination with a heat shock protein 90 inhibitor (HSP90i) (STA-9090 (ganetespib)).

FIG. 6 is an illustration of a Sickle Cell Disease mutation (GAG->GTG) in the human beta globin gene, relative to the ATG transcriptional start site and exemplary tcPNAs.

FIG. 7A is a bar graph showing the results of a MQAE (N-(Ethoxycarbonylmethyl)-6-Methoxyquinolinium Bromide) assay (delta(AFU)/(delta(Time (sec)) measuring chloride flux for negative control CFBE cells; CFBE cells treated with blank nanoparticles, PNA2-loaded nanoparticles, PNA2-loaded nanoparticles with an MPG peptide, γPNA2-loaded nanoparticles; and untreated positive control wildtype 16HBE14o-cells. FIG. 7B is a dot pot showing nasal potential difference (NPD) (pretreatment, after treatment with γPNA2-loaded nanoparticles, and after treatment with blank nanoparticles) measured using a non-invasive assay used to detect chloride potential differences in vivo.

FIG. 8 is an illustration of a mutation (G->A) in the CFTR gene (W1282X) relative to three exemplary tcPNAs.

FIG. 9 is an illustration of a mutation (G->T) in the CFTR gene (G542X) relative to three exemplary tcPNAs.

FIG. 10A is an illustration of Strategy for targeted correction of a β-globin gene containing SCD mutation (A->T) mutation and tcPNAs designed to bind to homopurine regions near the mutation. FIGS. 10B-10C are bar graphs showing hydrodynamic diameter of formulated PLGA nanoparticles measured using dynamic light scattering in PBS buffer (FIG. 10B) and zeta potential of formulated PLGA nanoparticles (FIG. 10C). Data in both graphs are presented as mean±s.e.m., n=3. FIGS. 10D-10E are bar graphs showing the results of deep-sequencing analysis to quantify the frequency of targeted gene editing in vivo in bone marrow cells of Berkley “Berk” mice (FIG. 10D) and Townes mice (FIG. 10E). Error bars indicate standard error of proportions.

FIGS. 11A-11D are graphs showing (FIG. 11A) survival to weaning (21 days) of IV and IA injected mice compared to untreated controls (untreated n=7 litters, IV n=4 litters, IA n=7 litters), data are mean s.e.m., statistical analysis by one-way ANOVA, p=0.5053; (FIG. 11B) long-term survival of IV and IA NP injected mice compared to untreated controls (n=15 females and n=15 males for each group), statistical analysis by Log-rank Mantel-Cox test, p=0.8490; weight of IV and IA NP injected female (FIG. 11C) and male (FIG. 11D) mice compared to untreated controls, (n=10 for each group), gray shaded region indicates the standard deviation of the control group. Data are shown as mean±s.d., statistical analysis by two-way ANOVA. FIG. 11E is a bar graph showing cytokine levels in plasma of E15.5 IV. For each cytokine bars represent, from left to right, untreated fetus compared to treated fetuses with PBS, blank NPs, or γtcPNA/DNA NP 48 h post treatment (n=3 for each group), data are mean s.e.m., statistical analysis by two-way ANOVA.

FIG. 12 is a graph showing cumulative release of nucleic acid from γPNA/DNA NPs over a period of 100 hours.

FIG. 13A is a bar graph showing blood hemoglobin levels of, from left to right for each time group of 6 and 10 weeks, untreated Hbb^{th-4}/Hbb⁺ mice; Hbb^{th-4}/Hbb⁺ mice treated with 300 mg/kg γtcPNA/DNA NP at E15.5; Hbb^{th-4}/Hbb⁺ mice treated with 400 mg/kg γtcPNA/DNA NP at E15.5; and wildtype B6 mice. Wild-type hemoglobin range is denoted by the gray shaded region between 11.0-15.1 g/dL, (n=6 for all groups), horizontal lines within the boxes indicate the mean, whiskers represent the range, statistical analysis by two-way ANOVA, **P<0.01, ***P<0.001, ****P<0.0001. FIG. 13B is gross images of spleens from untreated Hbb^{th-4}/Hbb⁺ mice, Hbb^{th-4}/Hbb⁺ mice 15-30 weeks after E15.5 12 mg/ml NP treatment and wild-type mice (untreated n=7, γPNA/DNA n=3, wild-type n=3).

FIG. 14A is a bar graph showing reticulotyes as percentages of total red blood cells (RBCs) of Hbb^{th-4}/Hbb⁺ mice 10 weeks after E15.5 IV γtcPNA/DNA NP treatment compared to untreated Hbb^{th-4}/Hbb⁺ and wild-type B6 mice, (n=6 for all groups), statistical analysis by two-way ANOVA, ****P<0.0001. FIG. 14B is a plot showing survival of Hbb^{th-4}/Hbb⁺ thalassemic mice treated in utero on day E15.5 by IV injection of γtcPNA/DNA NPs versus untreated age-matched controls (n=16 for both groups), with statistical analysis by Log-rank Mantel-Cox test, p=0.02. FIG. 14C is a bar graph showing ddPCR of genomic DNA from multiple tissues (collected after 15 weeks post-treatment) of mice treated on day E15.5 by IV injection of γtcPNA/DNA NPs versus untreated Hbb^{th-4}/Hbb⁺ controls, (n=3 for both groups). FIG. 14D-14E are plots showing how the expected fractional abundance of the wild-type allele (after QuantaSoft™ Software fit the fluorescence data after amplification to a Poisson distribution) was calculated using the ddPCR-quantified copies/μl of wild-type and beta-thal alleles in each control sample. The plots compare the ddPCR measured and expected fractional abundance of the wild-type allele in each sample. The observed correlation is linear. FIG. 14E is zoomed-in view of the lower range of FIG. 14D, indicated by a box on the plot of FIG. 14D. Error bars indicate the 95% confidence interval.

FIG. 15 is a bar graph showing long-term gene editing (9 months) in 654-eGFP mice (% gene editing in various tissue) either untreated, or treated with γPNA/DNA PLGA NPs either intravenously (vitelline vein) at E15.5 (15 μl of NPs resuspended at 9 mg/ml in 1×dPBS) or intra-amniotically at E16.5 (20 μl of NPs resuspended at 9 mg/ml in 1×dPBS), or treated with blank PLGA NPs.

FIG. 16A is a dot plot showing the presence of green fluorescent lung cells (indicating gene editing) detected by flow cytometry, in the 654-eGFP mouse model; nMFI=normalized mean fluorescent intensity (FIG. 16A). FIG. 16B is a bar graph showing confirmation of gene editing (% modification) by deep sequencing of e15.5 IV-treated lung 4 days post nanoparticle delivery. FIGS. 16C and 16D are representative 2D ddPCR plots of edited lungs four days post-intravenous in utero NP treatment. Looped dots labeled “empty” indicate empty droplets (no DNA template), looped dots labeled “beta-thal” indicate droplets loaded with unedited templates containing the beta-thalasemia 654-splice site mutation, looped dots labeled “edit” indicate droplets containing edited templates, and looped dots labeled “double positive” indicate droplets that contain both edited and un-edited templates. FIG. 16E is a dot plot showing the aggregate, quantitative ddPCR data. n=4 for all treatment groups.

FIGS. 17A-17E are 2D ddPCR plots. FIG. 17A illustrates the design of a ddPCR assay to detect murine CFTR gene editing. This 2D plot is the overlay of four samples (no template control (“no template”), ssDNA containing the F508del mutation (“F508del”), ssDNA containing the edited template (“edit”), and a sample containing both F508del and edited ssDNA (“double positive”)). FIG. 17B-17E are representative ddPCR 2D plots of no template control (17B), untreated gDNA (17C), gDNA extracted from the lung (17D) and nasal epithelium (17E) 8 months after E16.5 intra-amniotic treatment with PNA/DNA PLGA/PBAE/MPG NPs (20 μl of NPs resuspended to a concentration of 9 mg/ml in 1×dPBS). Looped dots labeled “no template” indicate empty droplets (no DNA template), looped dots labeled “F508del” indicate droplets containing the F508del mutation containing allele, looped dots labeled “edit” indicate droplets containing edited alleles, and looped dots labeled “double positive” indicate droplets that contain both edited and un-edited alleles. FIG. 17F is a dot plot showing the results of ddPCR of genomic DNA from multiple CF tissues 8 months (square), 15 months (circle) after E16.5 intra-amniotic NP delivery, and control CF gDNA. Each fetus was given 20 ul of NPs resuspended to a concentration of 9 mg/ml in 1×dPBS, (n=4).

FIG. 18 is a dot plot showing change in nasal potential difference (NPD) (mV) of untreated cystic fibrosis (CF) mice (black circles n=12), wildtype mice (n=7), and mice that received in utero nanoparticle (NP) treatment (n=4). NPD measurements were made four months after in utero NP treatment. Mice were treated in utero either intravenously with tcPNA/DNA PLGA NPs (black squares, n=2) or intra-amniotically with tcPNA/DNA PBAE/PLGA/MPG NPs (red diamonds, n=2). The gray shaded region indicates the range of wildtype NPD response.

FIG. 19 is a bar graph showing quantification of gene editing of the IVS2-654 mutation in bone marrow (BM) cells treated with PNA/DNA-loaded PLGA or 60% PDL PACE NPs after 48 h.

FIG. 20 is a graph showing the binding specificity (using defect-to-skin brightness ratio used as a surrogate for binding specificity) of fluorescent particles to the MMC defect for different particle compositions.

FIG. 21 is a graph showing the amount of rat/mouse basic fibroblast growth factor (bFGF) (pg/mL), obtained from ELISA, of non-injected MMC fetuses at different timepoints around the time of injection. Despite a decrease at E17.5, there appears to be an overall increase in bFGF levels toward full term, with a mean level of 67.3 pg/mL by E21.

FIG. 22 is a graph showing the amount of rat/mouse bFGF (pg/mL) in treated MMCs compared to control: 202.9+/−59 pg/mL vs 67.3+/−20.9 pg/mL, p=0.041.

FIG. 23 is a graph showing the amount of human bFGF in amniotic fluid injected with alginate particles delivering bFGF and stabilized with human serum albumin compared to control: 39.3+/−3.8 pg/mL vs 27.3+/−1.4 pg/mL, p=0.0046.

### DETAILED DESCRIPTION OF THE INVENTION

### I. Definitions

As used herein, “affinity tags” are defined herein as molecular species which form highly specific, non-covalent, physiochemical interactions with defined binding partners. Affinity tags which form highly specific, non-covalent, physiochemical interactions with one another are defined herein as “complementary”.

As used herein, “coupling agents” are defined herein as molecular entities which associate with polymeric particles and provide substrates that facilitate the modular assembly and disassembly of functional elements onto the particle. Coupling agents can be conjugated to affinity tags. Affinity tags allow for flexible assembly and disassembly of functional elements which are conjugated to affinity tags that form highly specific, noncovalent, physiochemical interactions with affinity tags conjugated to adaptor elements. Coupling agents can also be covalently coupled to functional elements in the absence of affinity tags.

As used herein, the term “isolated” describes a compound of interest (e.g., either a polynucleotide or a polypeptide) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein with respect to nucleic acids, the term “isolated” includes any non-naturally-occurring nucleic acid sequence, since such non-naturally-occurring sequences are not found in nature and do not have immediately contiguous sequences in a naturally-occurring genome.

As used herein, the term “host cell” refers to prokaryotic and eukaryotic cells into which a nucleic acid can be introduced.

As used herein, “transformed” and “transfected” encompass the introduction of a nucleic acid into a cell by one of a number of techniques known in the art.

As used herein, the phrase that a molecule “specifically binds” to a target refers to a binding reaction which is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Specific binding between two entities means an affinity of at least 10⁶, 10⁷, 10⁸, 10⁹, or 10¹⁰ M⁻¹. Affinities greater than 10⁸ M⁻¹ are preferred.

As used herein, “targeting molecule” is a substance which can direct a particle to a receptor site on a selected cell or tissue type, can serve as an attachment molecule, or serve to couple or attach another molecule. As used herein, “direct” refers to causing a molecule to preferentially attach to a selected cell or tissue type. This can be used to direct cellular materials, molecules, or drugs, as discussed below.

As used herein, the terms “antibody” or “immunoglobulin” are used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes a bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin. Exp. Immunol., 79:315-321 (1990); Kostelny, et al., J. Immunol., 148, 1547-1553 (1992).

As used herein, the terms “epitope” or “antigenic determinant” refer to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10, amino acids, in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke, et al., J. Inf Dis., 170:1110-19 (1994)), by antigen-dependent killing (cytotoxic T lymphocyte assay, Tigges, et al., J. Immunol., 156, 3901-3910) or by cytokine secretion.

As used herein, the term “small molecule,” as used herein, generally refers to an organic molecule that is less than about 2000 g/mol in molecular weight, less than about 1500 g/mol, less than about 1000 g/mol, less than about 800 g/mol, or less than about 500 g/mol. Small molecules are non-polymeric and/or non-oligomeric.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

The term “subject” or “patient” refers to any mammal who is the target of administration. Thus, the subject can be a human. The subject can be domesticated, agricultural, or wild animals. Domesticated animals include, for example, dogs, cats, rabbits, ferrets, guinea pigs, hamsters, pigs, monkeys or other primates, and gerbils. Agricultural animals include, for example, horses, cattle, pigs, sheep, rabbits, and goats. The term does not denote a particular age or sex. In some embodiments, the subject is an embryo or fetus.

### II. Particle Delivery Vehicles

Compositions and methods for in utero treatment of a fetus or embryo are provided. The methods can include injecting or infusing into the vitelline vein of a fetus or embryo, injecting or infusing into its amniotic sac (e.g., intraamniotic sac injection), or a combination thereof and a composition having an effective amount of an active agent. In some embodiments, the composition is the active agent. In some embodiments, an effective amount of the same or a different active agents are administered by injection or infusion into the vitelline vein and by injection or infusion into the amniotic sac. The active agent can be a therapeutic agent, a nutritional agent, a diagnostic agent, or a prophylactic agent. The active agent can be a small molecule, a protein, or a nucleic acid. The active agent can be an antisense agent or a gene editing composition.

The compositions can include a biodegradable or bioerodible material in which the active agent is embedded or encapsulated. Any of the active agents including, but not limited to, therapeutic, nutritional, diagnostic, prophylactic agents, etc., can be, but need not necessarily be, delivered to the target cells using a particle-based delivery vehicle. Thus, the active agent can be encapsulated and/or entrapped and/or dispersed in a particle(s). Compositions can include a plurality of particles having an active agent encapsulated and/or entrapped and/or dispersed therein, in a pharmaceutically-acceptable carrier, and formulated for infusion or injection into a vitelline artery or a vitelline vein or for intraamniotic sac injection.

The particles can be capable of controlled release of the active agent. The particles can be microparticle(s) and/or nanoparticle(s). The particles can include one or more polymers. One or more of the polymers can be a synthetic polymer. The particle or particles can be formed by, for example, single emulsion technique or double emulsion technique or nanoprecipitation.

In some embodiments, some of the compositions are packaged in particles and some are not. For example, a gene editing technology and/or donor oligonucleotide can be incorporated into particles while a co-administered potentiating factor is not. In some embodiments, a gene editing technology and/or donor oligonucleotide and a potentiating factor are both packaged in particles. Different compositions can be packaged in the same particles or different particles. For example, two or more active agents can be mixed and packaged together. In some embodiments, the different compositions are packaged separately into separate particles wherein the particles are similarly or identically composed and/or manufactured. In some embodiments, the different compositions are packaged separately into separate particles wherein the particles are differentially composed and/or manufactured.

The delivery vehicles can be nanoscale compositions, for example, 0.5 nm up to, but not including, about 1 micron. In some embodiments, and for some uses, the particles can be smaller, or larger. Thus, the particles can be microparticles, supraparticles, etc. For example, particle compositions can be between about 1 micron to about 1000 microns. Such compositions can be referred to as microparticulate compositions.

Nanoparticles generally refers to particles in the range of less than 0.5 nm up to, but not including 1,000 nm. In some embodiments, the nanoparticles have a diameter between 500 nm to less than 0.5 nm, or between 50 and 500 nm, or between 50 and 300 nm. Cellular internalization of polymeric particles can highly dependent upon their size, with nanoparticulate polymeric particles being internalized by cells with much higher efficiency than micoparticulate polymeric particles. For example, Desai, et al. have demonstrated that about 2.5 times more nanoparticles that are 100 nm in diameter are taken up by cultured Caco-2 cells as compared to microparticles having a diameter on 1 μM (Desai, et al., Pharm. Res., 14:1568-73 (1997)). Nanoparticles also have a greater ability to diffuse deeper into tissues in vivo.

In some embodiments, particularly those in which the particles need not be internalized by cells, the particles can be microparticles. Microparticle generally refers to a particle having a diameter, from about 1 micron to about 100 microns. The particles can also be from about 1 to about 50 microns, or from about 1 to about 30 microns, or from about 1 micron to about 10 microns. The microparticles can have any shape. Microparticles having a spherical shape may be referred to as “microspheres.”

Supraparticles are particles having a diameter above about 100 μm in size. For example, supraparticle may have a diameter of about 100 μm to about 1,000 μm in size.

The particles can have a mean particle size. Mean particle size generally refers to the statistical mean particle size (diameter) of the particles in the composition. Two populations can be said to have a substantially equivalent mean particle size when the statistical mean particle size of the first population of particles is within 20% of the statistical mean particle size of the second population of particles; more preferably within 15%, most preferably within 10%.

The weight average molecular weight can vary for a given polymer but is generally from about 1000 Daltons to 1,000,000 Daltons, 1000 Daltons to 500,000 Dalton, 1000 Daltons to 250,000 Daltons, 1000 Daltons to 100,000 Daltons, 5,000 Daltons to 100,000 Daltons, 5,000 Daltons to 75,000 Daltons, 5,000 Daltons to 50,000 Daltons, or 5,000 Daltons to 25,000 Daltons.

### A. Polymer

Particles are can be formed of one or more polymers. Exemplary polymers are discussed below. Copolymers such as random, block, or graft copolymers, or blends of the polymers listed below can also be used.

Functional groups on the polymer can be capped to alter the properties of the polymer and/or modify (e.g., decrease or increase) the reactivity of the functional group. For example, the carboxyl termini of carboxylic acid contain polymers, such as lactide- and glycolide-containing polymers, may optionally be capped, e.g., by esterification, and the hydroxyl termini may optionally be capped, e.g. by etherification or esterification.

Copolymers of PEG or derivatives thereof with any of the polymers described below may be used to make the polymeric particles. In certain embodiments, the PEG or derivatives may be located in the interior positions of the copolymer. Alternatively, the PEG or derivatives may locate near or at the terminal positions of the copolymer. For example, one or more of the polymers above can be terminated with a block of polyethylene glycol. In some embodiments, the core polymer is a blend of pegylated polymer and non-pegylated polymer, wherein the base polymer is the same (e.g., PLGA and PLGA-PEG) or different (e.g., PLGA-PEG and PLA). In certain embodiments, the microparticles or nanoparticles are formed under conditions that allow regions of PEG to phase separate or otherwise locate to the surface of the particles. The surface-localized PEG regions alone may perform the function of, or include, the surface-altering agent. In particular embodiments, the particles are prepared from one or more polymers terminated with blocks of polyethylene glycol as the surface-altering material.

### Release

In certain embodiments, the particles in amniotic space release therapeutic agents over at least 3 days, 5 days, 1 week, 2 weeks, 3 weeks, 1 month, 2 months, 3 months, or longer.

### Surface Charge

In certain embodiments, the particles possess a ζ-potential of between about 50 mV and about −50 mV, between about 30 mV and about −30 mV, or between about 10 mV and about −10 mV.

Some charges on the surface of the particles may facilitate binding with the MMC defect in amniotic space or binding to nucleic acids. Thus, the charge of the particles can be selected based on the active agent to be delivered, the disease to be treated, or a combination thereof.

In some embodiments, it may be desirable for the particles to have a negative charge, for example when delivering proteins such as growth factors. Thus, some or all of the polymers forming the particle can have a terminal moiety that imparts a negative charge to the particle. In some embodiments, the negatively charged moiety is a chemical modification to the polymer itself that imparts a negative charge to the polymer. In some embodiments, the negatively charge moiety is a separate, negatively-charged component that is conjugated to the polymer.

The terminal moiety that imparts a negative charge to the particles can be an acidic group or an anionic group. Examples of acidic groups include, but are not limited to, carboxylic acids, protonated sulfates, protonated sulfonates, protonated phosphates, singly- or doubly protonated phosphonates, and singly- or doubly protonated hydroxamates. The corresponding salts of these acidic groups form anionic groups such as carboxylates, sulfates, sulfonates, singly- or doubly deprotonated phosphates, singly- or doubly deprotonated phosphonates, and hydroxamates.

In some embodiments, the particles may be used as nucleic acid carriers. In these embodiments, the particles can be formed of one or more cationic polymers which complex with one or more nucleic acids which are negatively charged.

The cationic polymer can be any synthetic or natural polymer bearing at least two positive charges per molecule and having sufficient charge density and molecular size to bind to nucleic acid under physiological conditions (i.e., pH and salt conditions encountered within the body or within cells). In certain embodiments, the polycationic polymer contains one or more amine residues.

Suitable cationic polymers include, for example, polyethylene imine (PEI), polyallylamine, polyvinylamine, polyvinylpyridine, aminoacetalized poly(vinyl alcohol), acrylic or methacrylic polymers (for example, poly(N,N-dimethylaminoethylmethacrylate)) bearing one or more amine residues, polyamino acids such as polyornithine, polyarginine, and polylysine, protamine, cationic polysaccharides such as chitosan, DEAE-cellulose, and DEAE-dextran, and polyamidoamine dendrimers (cationic dendrimer), as well as copolymers and blends thereof. In some embodiments, the polycationic polymer is poly(amine-co-ester), poly(amine-co-amide) polymer, or poly(amine-co-ester-co-ortho ester).

Cationic polymers can be either linear or branched, can be either homopolymers or copolymers, and when containing amino acids can have either L or D configuration, and can have any mixture of these features. Preferably, the cationic polymer molecule is sufficiently flexible to allow it to form a compact complex with one or more nucleic acid molecules.

In some embodiments, the cationic polymer has a molecular weight of between about 5,000 Daltons and about 100,000 Daltons, more preferably between about 5,000 and about 50,000 Daltons, most preferably between about 10,000 and about 35,000 Daltons.

In particular embodiments, the particles include a hydrophobic polymer, poly(amine-co-ester), poly(amine-co-amide) polymer, or poly(amine-co-ester-co-ortho ester), and optionally, but a shell of, for example, PEG. The core-shell particles can be formed by a co-block polymer. Exemplary polymers are provided below.

### 1. Exemplary Polymers Hydrophobic Polymers

The polymer that forms the core of the particle may be any biodegradable or non-biodegradable synthetic or natural polymer. In a preferred embodiment, the polymer is a biodegradable polymer.

Particles are ideal materials for the fabrication of gene editing delivery vehicles: 1) control over the size range of fabrication, down to 100 nm or less, an important feature for passing through biological barriers; 2) reproducible biodegradability without the addition of enzymes or cofactors; 3) capability for sustained release of encapsulated, protected nucleic acids over a period in the range of days to months by varying factors such as the monomer ratios or polymer size, for example, the ratio of lactide to glycolide monomer units in poly(lactide-co-glycolide) (PLGA); 4) well-understood fabrication methodologies that offer flexibility over the range of parameters that can be used for fabrication, including choices of the polymer material, solvent, stabilizer, and scale of production; and 5) control over surface properties facilitating the introduction of modular functionalities into the surface.

Any number of biocompatible polymers can be used to prepare the particles. In one embodiment, the biocompatible polymer(s) is biodegradable. In another embodiment, the particles are non-degradable. In other embodiments, the particles are a mixture of degradable and non-degradable particles.

Examples of preferred biodegradable polymers include synthetic polymers that degrade by hydrolysis such as poly(hydroxy acids), such as polymers and copolymers of lactic acid and glycolic acid, other degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), and poly(amine-co-ester) polymers, such as those described in Zhou, et al., Nature Materials, 11(1):82-90 (2011), Tietjen, et al. Nature Communications, 8:191 (2017) doi:10.1038/s41467-017-00297-x, and WO 2013/082529, U.S. Published Application No. 2014/0342003, and PCT/US2015/061375.

Preferred natural polymers include alginate and other polysaccharides, collagen, albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Exemplary polymers include, but are not limited to, cyclodextrin-containing polymers, in particular cationic cyclodextrin-containing polymers, such as those described in U.S. Pat. No. 6,509,323,

In some embodiments, non-biodegradable polymers can be used, especially hydrophobic polymers. Examples of preferred non-biodegradable polymers include ethylene vinyl acetate, poly(meth) acrylic acid, copolymers of maleic anhydride with other unsaturated polymerizable monomers, poly(butadiene maleic anhydride), polyamides, copolymers and mixtures thereof, and dextran, cellulose and derivatives thereof.

Other suitable biodegradable and non-biodegradable polymers include, but are not limited to, polyanhydrides, polyamides, polycarbonates, polyalkylenes, polyalkylenes such as polyethylene and polypropylene, polyalkylene glycols such as poly(ethylene glycol) (PEG), polyalkylene oxides (PEO), polyalkylene terephthalates such as poly(ethylene terephthalate) and ethylene vinyl acetate polymer (EVA), polyvinyl alcohols, polyvinyl ethers, polyvinyl esters such as poly(vinyl acetate), polyethylene, polypropylene, poly(vinyl acetate), poly vinyl chloride, polystyrene, polyvinyl halides such as poly(vinyl chloride) (PVC), polyvinylpyrrolidone, polysiloxanes, polyvinylpyrrolidone, polymers of acrylic and methacrylic esters, polysiloxanes, polyurethanes and copolymers thereof, modified celluloses, alkyl cellulose, hydroxyalkyl celluloses, cellulose ethers, cellulose esters, nitro celluloses, hydroxypropylcellulose, cellulose acetate, cellulose propionate, cellulose acetate butyrate, cellulose acetate phthalate, carboxyethyl cellulose, cellulose triacetate, cellulose sulfate sodium salt, and polyacrylates such as poly(methyl methacrylate), poly(ethylmethacrylate), Poly(2-hydroxyethyl methacrylate) (pHEMA), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate). These materials may be used alone, as physical mixtures (blends), or as co-polymers.

The polymer may be a bioadhesive polymer that is hydrophilic or hydrophobic. Hydrophilic polymers include CARBOPOL™ (a high molecular weight, crosslinked, acrylic acid-based polymers such as those manufactured by NOVEON™), polycarbophil, cellulose esters, and dextran. polymers of acrylic acids, include, but are not limited to, poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”).

Release rate controlling polymers may be included in the polymer matrix or in the coating on the formulation. Examples of rate controlling polymers that may be used are hydroxypropylmethylcellulose (HPMC) with viscosities of either 5, 50, 100 or 4000 cps or blends of the different viscosities, ethylcellulose, methylmethacrylates, such as EUDRAGIT® RS100, EUDRAGIT® RL100, EUDRAGIT® NE 30D (supplied by Rohm America). Gastrosoluble polymers, such as EUDRAGIT® E100 or enteric polymers such as EUDRAGIT® L100-55D, L100 and S100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

These polymers can be obtained from sources such as Sigma Chemical Co., St. Louis, MO; Polysciences, Warrenton, PA; Aldrich, Milwaukee, WI; Fluka, Ronkonkoma, NY; and BioRad, Richmond, CA, or can be synthesized from monomers obtained from these or other suppliers using standard techniques.

In certain embodiments, the hydrophobic polymer is an aliphatic polyester. In preferred embodiments, the hydrophobic polymer is polyhydroxyester such as poly(lactic acid), poly(glycolic acid), or poly(lactic acid-co-glycolic acid).

Other polymers include, but are not limited to, polyalkyl cyanoacralate, polyamino acids such as poly-L-lysine (PLL), poly(valeric acid), and poly-L-glutamic acid, hydroxypropyl methacrylate (HPMA), polyorthoesters, poly(ester amides), poly(ester ethers), polydioxanone and its copolymers, polyhydroxyalkanoates, polypropylene fumarate, polyoxymethylene, poly(butyric acid), trimethylene carbonate, and polyphosphazenes.

The particles can be designed to release molecules to be encapsulated or attached over a period of days to weeks. Factors that affect the duration of release include pH of the surrounding medium (higher rate of release at pH 5 and below due to acid catalyzed hydrolysis of PLGA) and polymer composition. Aliphatic polyesters differ in hydrophobicity and that in turn affects the degradation rate. The hydrophobic poly (lactic acid) (PLA), more hydrophilic poly (glycolic acid) PGA and their copolymers, poly (lactide-co-glycolide) (PLGA) have different release rates. The degradation rate of these polymers, and often the corresponding drug release rate, can vary from days (PGA) to months (PLA) and is easily manipulated by varying the ratio of PLA to PGA.

In some preferred embodiments, the particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(8-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. For example, particles can also contain one or more polymer conjugates containing end-to-end linkages between the polymer and a targeting moiety or a detectable label. For example, a modified polymer can be a PLGA-PEG-peptide block polymer.

The in vivo stability/release of the particles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

A shell can also be formed of or contain a hyperbranched polymer (HP) with hydroxyl groups, such as a hyperbranched polyglycerol (HPG), hyperbranched peptides (HPP), hyperbranched oligonucleotides (HON), hyperbranched polysaccharides (HPS), and hyperbranched polyunsaturated or saturated fatty acids (HPF). The HP can be covalently bound to the one or more materials that form the core such that the hydrophilic HP is oriented towards the outside of the particles and the hydrophobic material oriented to form the core.

The HP coating can be modified to adjust the properties of the particles. For example, unmodified HP coatings impart stealth properties to the particles which resist non-specific protein absorption and are referred to as nonbioadhesive nanoparticles (NNPs). Alternatively, the hydroxyl groups on the HP coating can be chemically modified to form functional groups that react with functional groups on tissue or otherwise interact with tissue to adhere the particles to the tissue, cells, or extracellular materials, such as proteins. Such functional groups include, but are not limited to, aldehydes, amines, and O-substituted oximes. Particles with an HP coating chemically modified to form functional groups are referred to as bioadhesive nanoparticles (BNPs). The chemically modified HP coating of BNPs forms a bioadhesive corona of the particle surrounding the hydrophobic material forming the core. See, for example, WO 2015/172149, WO 2015/172153, WO 2016/183209, and U.S. Published Applications 2017/0000737 and 2017/0266119.

Particles can be formed of polymers fabricated from polylactides (PLA) and copolymers of lactide and glycolide (PLGA). These have established commercial use in humans and have a long safety record (Jiang, et al., Adv. Drug Deliv. Rev., 57(3):391-410); Aguado and Lambert, Immunobiology, 184(2-3):113-25 (1992); Bramwell, et al., Adv. Drug Deliv. Rev., 57(9):1247-65 (2005)). These polymers have been used to encapsulate siRNA (Yuan, et al., Jour. Nanosocience and Nanotechnology, 6:2821-8 (2006); Braden, et al., Jour. Biomed. Nanotechnology, 3:148-59 (2007); Khan, et al., Jour. Drug Target, 12:393-404 (2004); Woodrow, et al., Nature Materials, 8:526-533 (2009)). Murata, et al., J. Control. Release, 126(3):246-54 (2008) showed inhibition of tumor growth after intratumoral injection of PLGA microspheres encapsulating siRNA targeted against vascular endothelial growth factor (VEGF). However, these microspheres were too large to be endocytosed (35-45 μm) (Conner and Schmid, Nature, 422(6927):37-44 (2003)) and required release of the anti-VEGF siRNA extracellularly as a polyplex with either polyarginine or PEI before they could be internalized by the cell. These microparticles may have limited applications because of the toxicity of the polycations and the size of the particles. Nanoparticles (100-300 nm) of PLGA can penetrate deep into tissue and are easily internalized by many cells (Conner and Schmid, Nature, 422(6927):37-44 (2003)).

Exemplary particles are described in U.S. Pat. Nos. 4,883,666, 5,114,719, 5,601,835, 7,534,448, 7,534,449, 7,550,154, and 8,889,117, and U.S. Published Application Nos. 2009/0269397, 2009/0239789, 2010/0151436, 2011/0008451, 2011/0268810, 2014/0342003, 2015/0118311, 2015/0125384, 2015/0073041, Hubbell, et al., Science, 337:303-305 (2012), Cheng, et al., Biomaterials, 32:6194-6203 (2011), Rodriguez, et al., Science, 339:971-975 (2013), Hrkach, et al., Sci Transl Med., 4:128ra139 (2012), McNeer, et al., Mol Ther., 19:172-180 (2011), McNeer, et al., Gene Ther., 20:658-659 (2013), Babar, et al., Proc Natl Acad Sci USA, 109:E1695-E1704 (2012), Fields, et al., J Control Release 164:41-48 (2012), and Fields, et al., Advanced Healthcare Materials, 361-366 (2015).

### 2. Poly(Amine-Co-Esters), Poly(Amine-Co-Amides), and Poly(Amine-Co-Ester-Co-Ortho Esters)

The core of the particles can be formed of or contain one or more poly(amine-co-ester), poly(amine-co-amide), poly(amine-co-ester-co-ortho ester) or a combination thereof. In some embodiments, the particles are polyplexes. In some embodiments, the content of a hydrophobic monomer in the polymer is increased relative the content of the same hydrophobic monomer when used to form polyplexes. Increasing the content of a hydrophobic monomer in the polymer forms a polymer that can form solid core particles in the presence of nucleic acids. Unlike polyplexes, these particles are stable for long periods of time during incubation in buffered water, or serum, or upon administration (e.g., injection) into animals. They also provide for a sustained release of nucleic acids which leads to long term activity. In some aspects, the molecular weight of the polymer is less than 5 kDa, 7.5 kDa, 10 kDa, 20 kDa, or 25 kDa. In some forms the molecular weight of the polymer is between about 1 kDa and about 25 kDa, between about 1 kDa and about 10 kDa, between about 1 kDa and about 7.5 kDa.

The polymers can have the general formula:

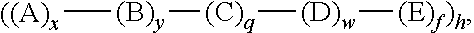

wherein A, B, C, D, and E independently include monomeric units derived from lactones (such as pentadecalactone), a polyfunctional molecule (such as N-methyldiethanolamine), a diacid or diester (such as diethylsebacate), an ortho ester, or polyalkylene oxide (such as polyethylene glycol). In some aspects, the polymers include at least a lactone, a polyfunctional molecule, and a diacid or diester monomeric units. In some aspects, the polymers include at least a lactone, a polyfunctional molecule, an ortho ester, and a diacid or diester monomeric units. In general, the polyfunctional molecule contains one or more cations, one or more positively ionizable atoms, or combinations thereof. The one or more cations are formed from the protonation of a basic nitrogen atom, or from quaternary nitrogen atoms.

In general, x, y, q, w, and f are independently integers from 0-1000, with the proviso that the sum (x+y+q+w+f) is greater than one. h is an integer from 1 to 1000.

In some forms, the percent composition of the lactone can be between about 30% and about 100%, calculated as the mole percentage of lactone unit vs. (lactone unit+diester/diacid). Expressed in terms of molar ratio, the lactone unit vs. (lactone unit+diester/diacid) content is between about 0.3 and about 1. Preferably, the number of carbon atoms in the lactone unit is between about 10 and about 24. In some embodiments, the number of carbon atoms in the lactone unit is between about 12 and about 16. In some embodiments, the number of carbon atoms in the lactone unit is 12 (dodecalactone), 15 (pentadecalactone), or 16 (hexadecalactone).

The molecular weight of the lactone unit in the polymer, the lactone unit's content of the polymer, or both, influences the formation of solid core particles.

Suitable polymers as well as particles and polyplexes formed therefrom are disclosed in WO 2013/082529, WO 2016/183217, U.S. Published Application No. 2016/0251477, U.S. Published Application No. 2015/0073041, U.S. Published Application No. 2014/0073041, and U.S. Pat. No. 9,272,043, each of which is specifically incorporated by reference in entirety.

For example, in some embodiments, the polymer includes a structure that has the formula:

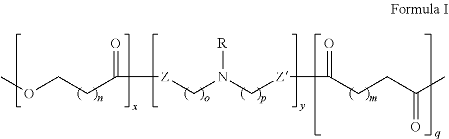

wherein n is an integer from 1-30, m, o, and p are independently an integer from 1-20, x, y, and q are independently integers from 1-1000, Z and Z′ are independently O or NR′, wherein R and R′ are independently hydrogen, substituted or unsubstituted alkyl, or substituted or unsubstituted aryl. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc. In particular embodiments, the values of x, y, and q are such that the weight average molecular weight of the polymer is greater than 5,000 Daltons. In some aspects, the molecular weight of the polymer is less than 5 kDa, 7.5 kDa, 10 kDa, 20 kDa, or 25 kDa. In some forms the molecular weight of the polymer is between about 1 kDa and about 25 kDa, between about 1 kDa and about 10 kDa, between about 1 kDa and about 7.5 kDa. The polymer can be prepared from one or more lactones, one or more amine-diols, triamines, or hydroxy diamines, and one or more diacids or diesters. In those embodiments where two or more different lactone, diacid or diester, and/or triamine, amine-diol, or hydroxy diamine monomers are used, the values of n, o, p, and/or m can be the same or different.

In some forms, the percent composition of the lactone unit is between about 30% and about 100%, calculated lactone unit vs. (lactone unit+diester/diacid). Expressed in terms of a molar ratio, the lactone unit vs. (lactone unit+diester/diacid) content is between about 0.3 and about 1, i.e., x/(x+q) is between about 0.3 and about 1. Preferably, the number of carbon atoms in the lactone unit is between about 10 and about 24, more preferably the number of carbon atoms in the lactone unit is between about 12 and about 16. Most preferably, the number of carbon atoms in the lactone unit is 12 (dodecalactone), 15 (pentadecalactone), or 16 (hexadecalactone).

In some embodiments, Z and Z′ are O. In some embodiments, Z is O and Z′ is NR′, or Z is NR′ and Z′ is O, wherein R′ is hydrogen, substituted or unsubstituted alkyl, or substituted or unsubstituted aryl. Examples of R′ include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

In some embodiments, Z and Z′ are O and n is an integer from 1-24, such 4, 10, 13, or 14.

In some embodiments, Z and Z′ are O, n is an integer from 1-24, such 4, 10, 13, or 14, and m is an integer from 1-10, such as 4, 5, 6, 7, or 8.

In some embodiments, Z and Z′ are O, n is an integer from 1-24, such 4, 10, 13, or 14, m is an integer from 1-10, such as 4, 5, 6, 7, or 8, and o and p are the same integer from 1-6, such 2, 3, or 4.

In some embodiments, Z and Z′ are O, n is an integer from 1-24, such 4, 10, 13, or 14, m is an integer from 1-10, such as 4, 5, 6, 7, or 8, and R is alkyl, such as methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, or aryl, such as phenyl, naphthalyl, anthracenyl, phenanthryl, chrysenyl, pyrenyl, tolyl, or xylyl.

In certain embodiments, n is 14 (e.g., pentadecalactone, PDL), m is 7 (e.g., diethylsebacate, DES), o and p are 2 (e.g., N-methyldiethanolamine, MDEA). In certain embodiments, n, m, o, and p are as defined above, and PEG is incorporated as a monomer.

In particular embodiments, the values of x, y, and q are such that the weight average molecular weight of the polymer is greater than 5,000 Daltons.

The polymer can be prepared from one or more substituted or unsubstituted lactones, one or more substituted or unsubstituted amine-diols (Z and Z′=O), triamines (Z and Z′═NR′), or hydroxy-diamines (Z=O, and Z′=NR′, or vice versa) and one or more substituted or unsubstituted diacids or diesters. In those embodiments where two or more different lactone, diacid or diester, and/or triamine, amine-diol, or hydroxy diamine monomers are used, than the values of n, o, p, and/or m can be the same or different.

The monomer units can be substituted at one or more positions with one or more substituents. Exemplary substituents include, but are not limited to, alkyl groups, cyclic alkyl groups, alkene groups, cyclic alkene groups, alkynes, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

The polymer is preferably biocompatible. Readily available lactones of various ring sizes are known to possess low toxicity: for example, polyesters prepared from small lactones, such as poly(caprolactone) and poly(p-dioxanone) are commercially available biomaterials which have been used in clinical applications. Large (e.g., C₁₆-C₂₄) lactones and their polyester derivatives are natural products that have been identified in living organisms, such as bees. Lactones containing ring carbon atoms between 16 and 24 are specifically contemplated and disclosed.

In other embodiments, the polymer is biocompatible and biodegradable. The nucleic acid(s) encapsulated by and/or associated with the particles can be released through different mechanisms, including diffusion and degradation of the polymeric matrix. The rate of release can be controlled by varying the monomer composition of the polymer and thus the rate of degradation. For example, if simple hydrolysis is the primary mechanism of degradation, increasing the hydrophobicity of the polymer may slow the rate of degradation and therefore increase the time period of release. In all case, the polymer composition is selected such that an effective amount of nucleic acid(s) is released to achieve the desired purpose/outcome.

The polymers can further include one or more blocks of an alkylene oxide, such as polyethylene oxide, polypropylene oxide, and/or polyethylene oxide-co-polypropylene oxide. The structure of a PEG-containing polymer is shown below:

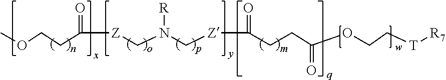

wherein n is an integer from 1-30, m, o, and p are independently an integer from 1-20, x, y, q, and w are independently integers from 1-1000, Z and Z′ are independently O or NR′, wherein R and R′ are independently hydrogen, substituted or unsubstituted alkyl, or substituted or unsubstituted aryl, wherein T is oxygen or is absent, and wherein R₇ is hydrogen, alkyl, substituted alkyl, aryl, substituted alkyl, cycloalkyl, substituted cycloalkyl, maleimide, amine, thiol, N-hydroxysuccinimide ester, azide, acrylate, methacrylate, alkyne, hydroxide, or isocynate. In particular embodiments, the values of x, y, q, and w are such that the weight average molecular weight of the polymer is greater than 5,000 Daltons. In some aspects, the molecular weight of the polymer is less than 5 kDa, 7.5 kDa, 10 kDa, 20 kDa, or 25 kDa. In some forms the molecular weight of the polymer is between about 1 kDa and about 25 kDa, between about 1 kDa and about 10 kDa, between about 1 kDa and about 7.5 kDa. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

The structure of a PEG-containing copolymer is shown below:

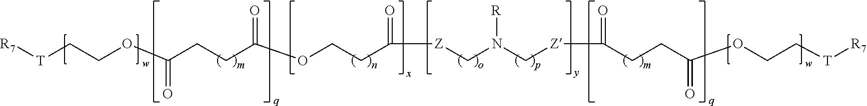

wherein n is an integer from 1-30, m, o, and p are independently an integer from 1-20, x, y, q, and w are independently integers from 1-1000, Z and Z′ are independently O or NR′, wherein R and R′ are independently hydrogen, substituted or unsubstituted alkyl, or substituted or unsubstituted aryl, wherein T is oxygen or is absent, and wherein R₇ is hydrogen, alkyl, substituted alkyl, aryl, substituted alkyl, cycloalkyl, substituted cycloalkyl, maleimide, amine, thiol, N-hydroxysuccinimide ester, azide, acrylate, methacrylate, alkyne, hydroxide, or isocynate. In particular embodiments, the values of x, y, q, and w are such that the weight average molecular weight of the polymer is greater than 5,000 Daltons. In some aspects, the molecular weight of the polymer is less than 5 kDa, 7.5 kDa, 10 kDa, 20 kDa, or 25 kDa. In some forms the molecular weight of the polymer is between about 1 kDa and about 25 kDa, between about 1 kDa and about 10 kDa, between about 1 kDa and about 7.5 kDa. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

The blocks of polyalkylene oxide can located at the termini of the polymer (i.e., by reacting PEG having one hydroxy group blocked, for example, with a methoxy group), within the polymer backbone (i.e., neither of the hydroxyl groups are blocked), or combinations thereof.

In particular embodiments, the synthetic polymer includes polymers or copolymers of lactic acid, glycolic acid, degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), poly(amine-co-ester) polymers, or a combination of any two or more of the foregoing. In specific embodiments, the particles are nanoparticles formed of PLGA poly(lactic-co-glycolic acid) (PLGA), a blend of PLGA and poly(beta-amino) esters (PBAEs) (e.g., about 5 and about 25 percent PBAE (wt %)), or poly(amine-co-ester) (PACE). In some embodiments, these particles are utilized for intracellular delivery of gene editing compositions such as peptide nucleic acids alone or in combination with donor oligonucleotides.

In other particular embodiments, the particles are formed of hydrogel type materials such as alginate, chitosan, poly(HEMA) and other acrylate polymers and copolymers. In specific embodiments, the particles are microparticles formed of alginate. In some embodiments, these particles are utilized for extracellular delivery (e.g., paracrine delivery) of a growth factor such a FGF.

### B. Polycations

In some embodiments, the nucleic acids are complexed to polycations to increase the encapsulation efficiency of the nucleic acids into the particles. The term “polycation” refers to a compound having a positive charge, preferably at least 2 positive charges, at a selected pH, preferably physiological pH. Polycationic moieties have between about 2 to about 15 positive charges, preferably between about 2 to about 12 positive charges, and more preferably between about 2 to about 8 positive charges at selected pH values.

Many polycations are known in the art. Suitable constituents of polycations include basic amino acids and their derivatives such as arginine, asparagine, glutamine, lysine and histidine; cationic dendrimers; and amino polysaccharides. Suitable polycations can be linear, such as linear tetralysine, branched or dendrimeric in structure.

Exemplary polycations include, but are not limited to, synthetic polycations based on acrylamide and 2-acrylamido-2-methylpropanetrimethylamine, poly(N-ethyl-4-vinylpyridine) or similar quartemized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, and polypeptides such as protamine, the histone polypeptides, polylysine, polyarginine and polyornithine.

In one embodiment, the polycation is a polyamine. Polyamines are compounds having two or more primary amine groups. In a preferred embodiment, the polyamine is a naturally occurring polyamine that is produced in prokaryotic or eukaryotic cells. Naturally occurring polyamines represent compounds with cations that are found at regularly-spaced intervals and are therefore particularly suitable for complexing with nucleic acids. Polyamines play a major role in very basic genetic processes such as DNA synthesis and gene expression. Polyamines are integral to cell migration, proliferation and differentiation in plants and animals. The metabolic levels of polyamines and amino acid precursors are critical and hence biosynthesis and degradation are tightly regulated. Suitable naturally occurring polyamines include, but are not limited to, spermine, spermidine, cadaverine and putrescine. In a preferred embodiment, the polyamine is spermidine.

In another embodiment, the polycation is a cyclic polyamine. Cyclic polyamines are known in the art and are described, for example, in U.S. Pat. No. 5,698,546, WO 1993/012096 and WO 2002/010142. Exemplary cyclic polyamines include, but are not limited to, cyclen.

Spermine and spermidine are derivatives of putrescine (1,4-diaminobutane), which is produced from L-ornithine by action of ODC (ornithine decarboxylase). L-ornithine is the product of L-arginine degradation by arginase. Spermidine is a triamine structure that is produced by spermidine synthase (SpdS) which catalyzes monoalkylation of putrescine (1,4-diaminobutane) with decarboxylated S-adenosylmethionine (dcAdoMet) 3-aminopropyl donor. The formal alkylation of both amino groups of putrescine with the 3-aminopropyl donor yields the symmetrical tetraamine spermine. The biosynthesis of spermine proceeds to spermidine by the effect of spermine synthase (SpmS) in the presence of dcAdoMet. The 3-aminopropyl donor (dcAdoMet) is derived from S-adenosylmethionine by sequential transformation of L-methionine by methionine adenosyltransferase followed by decarboxylation by AdoMetDC (S-adenosylmethionine decarboxylase). Hence, putrescine, spermidine and spermine are metabolites derived from the amino acids L-arginine (L-ornithine, putrescine) and L-methionine (dcAdoMet, aminopropyl donor).

In some embodiments, the particles themselves are a polycation (e.g., a blend of PLGA and poly(beta amino ester).

### C. Coupling Agents or Ligands

The external surface of the polymeric particles may be modified by conjugating to, or incorporating into, the surface of the particle a coupling agent or ligand.

In a preferred embodiment, the coupling agent is present in high density on the surface of the particle. As used herein, “high density” refers to polymeric particles having a high density of ligands or coupling agents, which is preferably in the range of 1,000 to 10,000,000, more preferably 10,000-1,000,000 ligands per square micron of particle surface area. This can be measured by fluorescence staining of dissolved particles and calibrating this fluorescence to a known amount of free fluorescent molecules in solution.

Coupling agents associate with the polymeric particles and provide substrates that facilitate the modular assembly and disassembly of functional elements to the particles. Coupling agents or ligands may associate with particles through a variety of interactions including, but not limited to, hydrophobic interactions, electrostatic interactions and covalent coupling.

In a preferred embodiment, the coupling agents are molecules that match the polymer phase hydrophile-lipophile balance. Hydrophile-lipophile balances range from 1 to 15. Molecules with a low hydrophile-lipophile balance are more lipid loving and thus tend to make a water in oil emulsion while those with a high hydrophile-lipophile balance are more hydrophilic and tend to make an oil in water emulsion. Fatty acids and lipids have a low hydrophile-lipophile balance below 10.

Any amphiphilic polymer with a hydrophile-lipophile balance in the range 1-10, more preferably between 1 and 6, most preferably between 1 and up to 5, can be used as a coupling agent. Examples of coupling agents which may associate with polymeric particles via hydrophobic interactions include, but are not limited to, fatty acids, hydrophobic or amphipathic peptides or proteins, and polymers. These classes of coupling agents may also be used in any combination or ratio. In a preferred embodiment, the association of adaptor elements with particles facilitates a prolonged presentation of functional elements, which can last for several weeks.

Coupling agents can also be attached to polymeric particles through covalent interactions through various functional groups. Functionality refers to conjugation of a molecule to the surface of the particle via a functional chemical group (carboxylic acids, aldehydes, amines, sulfhydryls and hydroxyls) present on the surface of the particle and present on the molecule to be attached.

Functionality may be introduced into the particles in two ways. The first is during the preparation of the particles, for example during the emulsion preparation of particles by incorporation of stabilizers with functional chemical groups. Suitable stabilizers include hydrophobic or amphipathic molecules that associate with the outer surface of the particles.

A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This second class also includes a process whereby amphiphilic molecules such as fatty acids, lipids or functional stabilizers may be passively adsorbed and adhered to the particle surface, thereby introducing functional end groups for tethering to ligands.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a molecule such as a protein. The reaction is an N-nucleophilic substitution and results in a stable N-alkylcarbamate linkage of the molecule to the polymer. The “coupling” of the molecule to the “activated” polymer matrix is maximal in the pH range of 9-10 and normally requires at least 24 hrs. The resulting molecule-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of molecules in a totally aqueous environment at the physiological pH of 7.0. Briefly, EDAC and sulfo-NHS form an activated ester with the carboxylic acid groups of the polymer which react with the amine end of a molecule to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the molecule-polymer complex.

By using either of these protocols it is possible to “activate” almost all polymers containing either hydroxyl or carboxyl groups in a suitable solvent system that will not dissolve the polymer matrix.

A useful coupling procedure for attaching molecules with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of molecules and polymers with double bonds, including the use of UV crosslinking, may be used for attachment of molecules to the polymer.

In one embodiment, coupling agents can be conjugated to affinity tags. Affinity tags are any molecular species which form highly specific, noncovalent, physiochemical interactions with defined binding partners. Affinity tags which form highly specific, noncovalent, physiochemical interactions with one another are defined herein as “complementary”. Suitable affinity tag pairs are well known in the art and include epitope/antibody, biotin/avidin, biotin/streptavidin, biotin/neutravidin, glutathione-S-transferase/glutathione, maltose binding protein/amylase and maltose binding protein/maltose. Examples of suitable epitopes which may be used for epitope/antibody binding pairs include, but are not limited to, HA, FLAG, c-Myc, glutatione-S-transferase, His₆, GFP, DIG, biotin and avidin. Antibodies (both monoclonal and polyclonal and antigen-binding fragments thereof) which bind to these epitopes are well known in the art.

Affinity tags that are conjugated to coupling agents allow for highly flexible, modular assembly and disassembly of functional elements which are conjugated to affinity tags which form highly specific, noncovalent, physiochemical interactions with complementary affinity tags which are conjugated to coupling agents. Adaptor elements may be conjugated with a single species of affinity tag or with any combination of affinity tag species in any ratio. The ability to vary the number of species of affinity tags and their ratios conjugated to adaptor elements allows for exquisite control over the number of functional elements which may be attached to the particles and their ratios.

In another embodiment, coupling agents are coupled directly to functional elements in the absence of affinity tags, such as through direct covalent interactions. Coupling agents can be covalently coupled to at least one species of functional element. Coupling agents can be covalently coupled to a single species of functional element or with any combination of species of functional elements in any ratio.

In a preferred embodiment, coupling agents are conjugated to at least one affinity tag that provides for assembly and disassembly of modular functional elements which are conjugated to complementary affinity tags. In a more preferred embodiment, coupling agents are fatty acids that are conjugated with at least one affinity tag. In a particularly preferred embodiment, the coupling agents are fatty acids conjugated with avidin or streptavidin. Avidin/streptavidin-conjugated fatty acids allow for the attachment of a wide variety of biotin-conjugated functional elements.

The coupling agents are preferably provided on, or in the surface of, particles at a high density. This high density of coupling agents allows for coupling of the polymeric particles to a variety of species of functional elements while still allowing for the functional elements to be present in high enough numbers to be efficacious.

### 1. Fatty Acids

The coupling agents may include fatty acids. Fatty acids may be of any acyl chain length and may be saturated or unsaturated. In a particularly preferred embodiment, the fatty acid is palmitic acid. Other suitable fatty acids include, but are not limited to, saturated fatty acids such as butyric, caproic, caprylic, capric, lauric, myristic, stearic, arachidic and behenic acid. Still other suitable fatty acids include, but are not limited to, unsaturated fatty acids such as oleic, linoleic, alpha-linolenic, arachidonic, eicosapentaenoic, docosahexaenoic and erucic acid.

### 2. Hydrophobic or Amphipathic Peptides

The coupling agents may include hydrophobic or amphipathic peptides. Preferred peptides should be sufficiently hydrophobic to preferentially associate with the polymeric particle over the aqueous environment. Amphipathic polypeptides useful as adaptor elements may be mostly hydrophobic on one end and mostly hydrophilic on the other end. Such amphipathic peptides may associate with polymeric particles through the hydrophobic end of the peptide and be conjugated on the hydrophilic end to a functional group.

### 3. Hydrophobic Polymers

Coupling agents may include hydrophobic polymers. Examples of hydrophobic polymers include, but are not limited to, polyanhydrides, poly(ortho)esters, and polyesters such as polycaprolactone.

### III. Therapeutic, Prophylactic, and Diagnostic Agent

The methods typically include in utero delivery of one or more therapeutic prophylactic, or diagnostic agents. Thus, in some embodiments, the particles have encapsulated therein, dispersed therein, complexed thereto and/or covalently or non-covalently associated with the surface one or more therapeutic prophylactic, or diagnostic agents. The therapeutic prophylactic, or diagnostic agent can be a small molecule, protein, polysaccharide or saccharide, nucleic acid molecule and/or lipid.

### A. Growth Factors

In some embodiments, the agent is a growth factor. Growth factors typically act as signaling molecules between cells. Examples are cytokines and hormones that bind to specific receptors on the surface of their target cells. They often promote cell differentiation and maturation, which varies between growth factors. For example, bone morphogenetic proteins stimulate bone cell differentiation, while fibroblast growth factors and vascular endothelial growth factors stimulate blood vessel differentiation. Exemplary growth factors include, but are not limited to, Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Colony-stimulating factors, Macrophage colony-stimulating factor (m-CSF), Granulocyte colony-stimulating factor (G-CSF), Granulocyte macrophage colony-stimulating factor (GM-CSF), Epidermal growth factor (EGF), Ephrins, Erythropoietin (EPO), Fibroblast growth factors (FGF, and FGF1-23), Foetal Bovine Somatotrophin (FBS), GDNF family of ligands, Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factors, Insulin-like growth factor-1 (IGF-1), Insulin-like growth factor-2 (IGF-2), Interleukins (e.g., IL-1, 2, 3, 4, 5, 6, 7), Keratinocyte growth factor (KGF), Migration-stimulating factor (MSF), Macrophage-stimulating protein (MSP), also known as hepatocyte growth factor-like protein (HGFLP), Myostatin (GDF-8), Neuregulins (e.g, Neurgeulin 1-4), Neurotrophins, Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factors, Transforming growth factor alpha (TGF-α), Transforming growth factor beta (TGF-β), Tumor necrosis factor-alpha (TNF-α), Vascular endothelial growth factor (VEGF), and Wnt signaling pathway modulators.

Basic Fibroblast Growth Factor (bFGF or FGF2)

In some embodiment, the growth factor is an FGF. The mammalian fibroblast growth factor (FGF) family consists of 22 structurally similar polypeptides that are present in most multicellular organisms and exert diverse biological effects. Members of the FGF family are active in many embryological and adult physiological processes, and FGFs have been shown to play a part in many developmental, neoplastic, metabolic, and neurological diseases. FGFs can work via paracrine, intracrine, and endocrine signaling pathways. One of the prototypical FGFs, FGF2 or “basic FGF” (bFGF), is an 18 kDa polypeptide with 155 amino acids that works primarily by paracrine signaling and plays an important role in all four phases of wound healing: hemostasis; inflammation; proliferation; remodeling. bFGF is a potent mitogen that stimulates the migration, proliferation, and differentiation of cells of mesenchymal and neurectodermal origin, such as keratinocytes, fibroblasts, melanocytes, and endothelial cells. For the purpose of treating fetal MMC, it the ability of bFGF to stimulate growth and proliferation of keratinocytes and fibroblasts, vital participants in new skin formation, that makes it a logical therapeutic candidate.

Studies have proven the ability of bFGF to induce new skin and blood vessel formation in rats and sheep. One group in particular has conducted multiple experiments with gelatin sponges impregnated with human recombinant bFGF in rat and sheep models of MMC. Human bFGF is 97% identical to mouse and rat bFGF, and despite the 3% of difference in amino acid sequence, the successful use of human bFGF in rodents in prior studies indicates that there is sufficient cross-reactivity between human and rat bFGF in vivo. This group showed that new vessel and skin growth occurred in the immediate area where BFGF scaffolds or sponges were applied, which is consistent with bFGF's function as a paracrine signaling molecule. Alginate microparticles have been successfully used for encapsulation and controlled delivery of growth factor protein. The Examples below show that intra-amniotic injection of various biodegradable and biocompatible microparticles is safe for the delivery of fluorescent dyes and nucleotides, and FGF delivered in this manner can be used to treat MMC.

### B. Gene Editing

In some embodiments, the active agent is one or more nucleic acids. The nucleic acid can alter, correct, or replace an endogenous nucleic acid sequence. The nucleic acid can be used to, for example, treat cancers, diseases and disorders, and correct defects in genes.

Gene therapy is a technique for correcting defective genes responsible for disease development. Researchers may use one of several approaches for correcting faulty genes:

A normal gene may be inserted into a nonspecific location within the genome to replace a nonfunctional gene. This approach is most common.

An abnormal gene can be swapped for a normal gene through homologous recombination.

The abnormal gene can be repaired through selective reverse mutation, which returns the gene to its normal function.

The regulation (the degree to which a gene is turned on or off) of a particular gene could be altered.

The nucleic acid can be a DNA, RNA, a chemically modified nucleic acid, or combinations thereof. Gene editing technologies are discussed in more detail below.

### C. Small Molecule Compounds

In some embodiments, the active agent is one or more small molecules. Exemplary classes of small molecule therapeutic agents include, but are not limited to, analgesics, anti-inflammatory drugs, antipyretics, antidepressants, antiepileptics, antiopsychotic agents, neuroprotective agents, anti-proliferatives, such as anti-cancer agent, anti-infectious agents, such as antibacterial agents and antifungal agents, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics.

### D. Diagnostic Agents

In some embodiments, the active agent is one or diagnostic agents. Exemplary diagnostic materials include paramagnetic molecules, fluorescent compounds, magnetic molecules, and radionuclides. Suitable diagnostic agents include, but are not limited to, x-ray imaging agents and contrast media. Radionuclides also can be used as imaging agents. Examples of other suitable contrast agents include gases or gas emitting compounds, which are radioopaque. Nanoparticles can further include agents useful for determining the location of administered particles. Agents useful for this purpose include fluorescent tags, radionuclides and contrast agents.

For those embodiments where the one or more therapeutic, prophylactic, and/or diagnostic agents are encapsulated within a polymeric nanoparticle and/or associated with the surface of the nanoparticle, the percent drug loading is from about 1% to about 80%, from about 1% to about 50%, preferably from about 1% to about 40% by weight, more preferably from about 1% to about 20% by weight, most preferably from about 1% to about 10% by weight. The ranges above are inclusive of all values from 1% to 80%. For those embodiments where the agent is associated with the surface of the particle, the percent loading may be higher since the amount of drug is not limited by the methods of encapsulation. In some embodiments, the agent to be delivered may be encapsulated within a nanoparticle and associated with the surface of the particle. Nutraceuticals can also be incorporated. These may be vitamins, supplements such as calcium or biotin, or natural ingredients such as plant extracts or phytohormones.

### E. Excipient

Excipients may be included in the particle formulations to enhance the stability, solubility, and/or controlled release manner of encapsulated therapeutic agents. An exemplary excipient to stabilize protein agents is human serum albumin. In other forms, trehalose may be used as a stabilizing agent for proteins.

### IV. Gene Editing Technology

In some embodiments, the therapeutic, prophylactic or diagnostic agent is, or encodes, a gene editing technology. Gene editing technologies can be used alone or in combination with a potentiating agent and/or other active agents. Exemplary gene editing technologies include, but are not limited to, triplex-forming, pseudocomplementary oligonucleotides, CRISPR/Cas, zinc finger nucleases, and TALENs, each of which are discussed in more detail below. Some gene editing technologies are used in combination with a donor oligonucleotide. In some embodiments, the gene editing technology is the donor oligonucleotide, which can be used alone to modify genes. Strategies include, but are not limited to, small fragment homologous replacement (e.g., polynucleotide small DNA fragments (SDFs)), single-stranded oligodeoxynucleotide-mediated gene modification (e.g., ssODN/SSOs) and other described in Sargent, Oligonucleotides, 21(2): 55-75 (2011)), and elsewhere. Other suitable gene editing technologies include, but are not limited to intron encoded meganucleases that are engineered to change their target specificity. See, e.g., Arnould, et al., Protein Eng. Des. Sel., 24(1-2):27-31 (2011)).

In some embodiments, the gene editing composition does not modify a target sequence within a maternal genome. In some embodiments, the target sequence of the fetal or embryonic genome and the target sequence of maternal genome are identical. In some embodiments they are not identical. The fetal or embryonic genome and the maternal genome can be isolated, derived, or obtained from genetically-related or genetically un-related individuals. The fetal or embryonic genome can include one or more mutations in a coding sequence or a non-coding sequence corresponding to a target gene that either indicates the fetus or embryo is at risk of developing a disease or disorder or that indicates that the fetus has a disease or disorder. The coding sequence or a non-coding sequence corresponding to the target gene can include the target sequence. The coding sequence corresponding to the target gene can include one or more exon(s) encoding a product of the target gene. The non-coding sequence corresponding to the target gene can include one or more transcriptional regulator(s), enhancer(s), superenhancer(s), intron(s), and regulatory RNAs that selectively bind a transcript of the target gene. The one or more transcriptional regulator(s) can include a sequence encoding a promoter. The one or more regulatory RNAs that selectively bind a transcript of the target gene comprise one or more miRNA(s). The mutation can include a substitution, an insertion, a deletion, an indel, an inversion, a frameshift, or a transposition. The mutation can be a transcriptional or translational truncation, altered transcriptional splicing, early termination of transcription or translation, variant transcriptional regulation or variant epigenetic regulation.

In some embodiments, the gene editing composition modifies a target sequence within a genome by reducing or preventing expression of the target sequence. The gene editing composition can induce single-stranded or double-stranded breaks in the target sequence. The gene editing composition can induce formation of a triplex within the target sequence.

Gene editing compositions include CRISPR systems, zinc finger nucleases (ZFN), transcription activator-like effector nucleases (TALEN), small fragment homologous replacement. The gene editing composition can be a triplex forming composition. The gene editing composition can be a pseudocomplementary oligonucleotide or PNA oligomer.

### A. Triplex-Forming Molecules (TFMs)

### 1. Compositions

Compositions containing “triplex-forming molecules,” that bind to duplex DNA in a sequence-specific manner to form a triple-stranded structure include, but are not limited to, triplex-forming oligonucleotides (TFOs), peptide nucleic acids (PNA), and “tail clamp” PNA (tcPNA). The triplex-forming molecules can be used to induce site-specific homologous recombination in mammalian cells when combined with donor DNA molecules. The donor DNA molecules can contain mutated nucleic acids relative to the target DNA sequence. This is useful to activate, inactivate, or otherwise alter the function of a polypeptide or protein encoded by the targeted duplex DNA. Triplex-forming molecules include triplex-forming oligonucleotides and peptide nucleic acids (PNAs). Triplex forming molecules are described in U.S. Pat. Nos. 5,962,426, 6,303,376, 7,078,389, 7,279,463, 8,658,608, U.S. Published Application Nos. 2003/0148352, 2010/0172882, 2011/0268810, 2011/0262406, 2011/0293585, and published PCT application numbers WO 1995/001364, WO 1996/040898, WO 1996/039195, WO 2003/052071, WO 2008/086529, WO 2010/123983, WO 2011/053989, WO 2011/133802, WO 2011/13380, Rogers, et al., Proc Natl Acad Sci USA, 99:16695-16700 (2002), Majumdar, et al., Nature Genetics, 20:212-214 (1998), Chin, et al., Proc Natl Acad Sci USA, 105:13514-13519 (2008), and Schleifman, et al., Chem Biol., 18:1189-1198 (2011). As discussed in more detail below, triplex forming molecules are typically single-stranded oligonucleotides that bind to polypyrimidine:polypurine target motif in a double stranded nucleic acid molecule to form a triple-stranded nucleic acid molecule. The single-stranded oligonucleotide/oligomer typically includes a sequence substantially complementary to the polypurine strand of the polypyrimidine:polypurine target motif.

a. Triplex-Forming Oligonucleotides (TFOs)

Triplex-forming oligonucleotides (TFOs) are defined as oligonucleotides which bind as third strands to duplex DNA in a sequence specific manner. The oligonucleotides are synthetic or isolated nucleic acid molecules which selectively bind to or hybridize with a predetermined target sequence, target region, or target site within or adjacent to a human gene so as to form a triple-stranded structure.

Preferably, the oligonucleotide is a single-stranded nucleic acid molecule between 7 and 40 nucleotides in length, most preferably 10 to 20 nucleotides in length for in vitro mutagenesis and 20 to 30 nucleotides in length for in vivo mutagenesis. The nucleobase (sometimes referred to herein simply as “base”) composition may be homopurine or homopyrimidine. Alternatively, the nucleobase composition may be polypurine or polypyrimidine. However, other compositions are also useful.

The oligonucleotides are preferably generated using known DNA synthesis procedures. In one embodiment, oligonucleotides are generated synthetically. Oligonucleotides can also be chemically modified using standard methods that are well known in the art.

The nucleobase sequence of the oligonucleotides/oligomer is selected based on the sequence of the target sequence, the physical constraints imposed by the need to achieve binding of the oligonucleotide/oligomer within the major groove of the target region, and the need to have a low dissociation constant (K_{d}) for the oligo/target sequence complex. The oligonucleotides/oligomers have a nucleobase composition which is conducive to triple-helix formation and is generated based on one of the known structural motifs for third strand binding (e.g. Hoogsteen binding). The most stable complexes are formed on polypurine:polypyrimidine elements, which are relatively abundant in mammalian genomes. Triplex formation by TFOs can occur with the third strand oriented either parallel or anti-parallel to the purine strand of the nucleic acid duplex. In the anti-parallel, purine motif, the triplets are G.G:C and A.A:T, whereas in the parallel pyrimidine motif, the canonical triplets are C⁺.G:C and T.A:T. The triplex structures can be stabilized by one, two or three Hoogsteen hydrogen bonds (depending on the nucleobase) between the bases in the TFO strand and the purine strand in the duplex. A review of base compositions and binding properties for third strand binding oligonucleotides and/or peptide nucleic acids is provided in, for example, U.S. Pat. No. 5,422,251, Bentin et al., Nucl. Acids Res., 34(20): 5790-5799 (2006), and Hansen et al., Nucl. Acids Res., 37(13): 4498-4507 (2009).

Preferably, the oligonucleotide/oligomer binds to or hybridizes to the target sequence under conditions of high stringency and specificity. Most preferably, the oligonucleotides/oligomers bind in a sequence-specific manner within the major groove of duplex DNA. Reaction conditions for in vitro triple helix formation of an oligonucleotide/oligomer to a double stranded nucleic acid sequence vary from oligo to oligo, depending on factors such as polymer length, the number of G:C and A:T base pairs, and the composition of the buffer utilized in the hybridization reaction. An oligonucleotide substantially complementary, based on the third strand binding code, to the target region of the double-stranded nucleic acid molecule is preferred.

As used herein, a triplex forming molecule is said to be substantially complementary to a target region when the oligonucleotide has a nucleobase composition which allows for the formation of a triple-helix with the target region. As such, an oligonucleotide/oligomer can be substantially complementary to a target region even when there are non-complementary bases present in the oligonucleotide/oligomer. As stated above, there are a variety of structural motifs available which can be used to determine the nucleobase sequence of a substantially complementary oligonucleotide/oligomer.

b. Peptide Nucleic Acids (PNA)

In another embodiment, the triplex-forming molecules are peptide nucleic acids (PNAs). Peptide nucleic acids can be considered polymeric molecules in which the sugar phosphate backbone of an oligonucleotide has been replaced in its entirety by repeating substituted or unsubstituted N-(2-aminoethyl)-glycine residues that are linked by amide bonds. The various nucleobases are linked to the backbone by methylene carbonyl linkages. PNAs maintain spacing of the nucleobases in a manner that is similar to that of an oligonucleotides (DNA or RNA), but because the sugar phosphate backbone has been replaced, classic (unsubstituted) PNAs are achiral and neutrally charged molecules. Peptide nucleic acids are composed of peptide nucleic acid residues (sometimes referred to as ‘residues’). The nucleobases can be any of the standard bases (uracil, thymine, cytosine, adenine and guanine) or any of the modified heterocyclic nucleobases described below.

PNAs can bind to DNA via Watson-Crick hydrogen bonds, but with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be two separate PNA molecules (see Bentin et al., Nucl. Acids Res., 34(20): 5790-5799 (2006) and Hansen et al., Nucl. Acids Res., 37(13): 4498-4507 (2009)), or two PNA molecules linked together by a linker of sufficient flexibility to form a single bis-PNA molecule (See: U.S. Pat. No. 6,441,130). In both cases, the PNA molecule(s) forms a triplex “clamp” with one of the strands of the target duplex while displacing the other strand of the duplex target. In this structure, one strand forms Watson-Crick base pairs with the DNA strand in the anti-parallel orientation (the Watson-Crick binding portion), whereas the other strand forms Hoogsteen base pairs to the DNA strand in the parallel orientation (the Hoogsteen binding portion). A homopurine strand allows formation of a stable PNA/DNA/PNA triplex. PNA clamps can form at shorter homopurine sequences than those required by triplex-forming oligonucleotides (TFOs) and also do so with greater stability.

Suitable molecules for use in linkers of bis-PNA molecules include, but are not limited to, 8-amino-3,6-dioxaoctanoic acid, referred to as an 0-linker, and 6-aminohexanoic acid. Poly(ethylene) glycol monomers can also be used in bis-PNA linkers. A bis-PNA linker can contain multiple linker residues in any combination of two or more of the foregoing.

PNAs can also include other positively charged moieties to increase the solubility of the PNA and increase the affinity of the PNA for duplex DNA. Commonly used positively charged moieties include the amino acids lysine and arginine (e.g., as additional substituents attached to the C- or N-terminus of the PNA oligomer (or a segment thereof) or as a side-chain modification of the backbone (see Huang et al., Arch. Pharm. Res. 35(3): 517-522 (2012) and Jain et al., JOC, 79(20): 9567-9577 (2014)), although other positively charged moieties may also be useful (See for Example: U.S. Pat. No. 6,326,479). In some embodiments, the PNA oligomer can have one or more ‘miniPEG’ side chain modifications of the backbone (see, for example, U.S. Pat. No. 9,193,758 and Sahu et al., JOC, 76: 5614-5627 (2011)).

Peptide nucleic acids are unnatural synthetic polyamides, prepared using known methodologies, generally as adapted from peptide synthesis processes.

c. Tail Clamp Peptide Nucleic Acids (tcPNA)

Although polypurine:polypyrimidine stretches do exist in mammalian genomes, it is desirable to target triplex formation in the absence of this requirement. In some embodiments such as PNA, triplex-forming molecules include a “tail” added to the end of the Watson-Crick binding portion. Adding additional nucleobases, known as a “tail” or “tail clamp”, to the Watson-Crick binding portion that bind to the target strand outside the triple helix further reduces the requirement for a polypurine:polypyrimidine stretch and increases the number of potential target sites. The tail is most typically added to the end of the Watson-Crick binding sequence furthest from the linker. This molecule therefore mediates a mode of binding to DNA that encompasses both triplex and duplex formation (Kaihatsu, et al., Biochemistry, 42(47):13996-4003 (2003); Bentin, et al., Biochemistry, 42(47):13987-95 (2003)). For example, if the triplex-forming molecules are tail clamp PNA (tcPNA), the PNA/DNA/PNA triple helix portion and the PNA/DNA duplex portion both produce displacement of the pyrimidine-rich strand, creating an altered helical structure that strongly provokes the nucleotide excision repair pathway and activating the site for recombination with a donor DNA molecule (Rogers, et al., Proc. Natl. Acad. Sci. U.S.A., 99(26):16695-700 (2002)).

Tails added to clamp PNAs (sometimes referred to as bis-PNAs) form tail-clamp PNAs (referred to as tcPNAs) that have been described by Kaihatsu, et al., Biochemistry, 42(47):13996-4003 (2003); Bentin, et al., Biochemistry, 42(47):13987-95 (2003). tcPNAs are known to bind to DNA more efficiently due to low dissociation constants. The addition of the tail also increases binding specificity and binding stringency of the triplex-forming molecules to the target duplex. It has also been found that the addition of a tail to clamp PNA improves the frequency of recombination of the donor oligonucleotide at the target site compared to PNA without the tail.

In some embodiments a PNA tail clamp system comprises:
a) optionally, a positively charged region having a positively charged amino acid subunit, e.g., a lysine subunit;
b) a first region comprising a plurality of PNA subunits having Hoogsteen homology with a target sequence;
c) a second region comprising a plurality of PNA subunits having Watson Crick homology binding with the target sequence;
d) a third region comprising a plurality of PNA subunits having Watson Crick homology binding with a tail target sequence;
e) optionally, a second positively charged region having a positively charged amino acid subunit, e.g., a lysine subunit.

In some embodiments, a linker is disposed between b) and c). In some embodiments, one or more PNA monomers of the tail claim is modified as disclosed herein.

d. PNA Modifications

PNAs can also include other positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues can be added to a bis-PNA linker or can be added to the carboxy or the N-terminus of a PNA strand. Common modifications to PNA are discussed in Sugiyama and Kittaka, Molecules, 18:287-310 (2013)) and Sahu, et al., J. Org. Chem., 76, 5614-5627 (2011), each of which are specifically incorporated by reference in their entireties, and include, but are not limited to, incorporation of charged amino acid residues, such as lysine at the termini or in the interior part of the oligomer; inclusion of polar groups in the backbone, carboxymethylene bridge, and in the nucleobases; chiral PNAs bearing substituents on the original N-(2-aminoethyl)glycine backbone; replacement of the original aminoethylglycyl backbone skeleton with a negatively-charged scaffold; conjugation of high molecular weight polyethylene glycol (PEG) to one of the termini; fusion of PNA to DNA to generate a chimeric oligomer, redesign of the backbone architecture, conjugation of PNA to DNA or RNA. These modifications improve solubility but often result in reduced binding affinity and/or sequence specificity. In some embodiments, the some or all of the PNA residues are modified at the gamma position in the polyamide backbone (γPNAs) as illustrated below (wherein “B” is a nucleobase and “R” is a substitution at the gamma position).

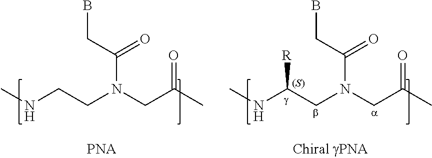

Substitution at the gamma position creates chirality and provides helical pre-organization to the PNA oligomer, yielding substantially increased binding affinity to the target DNA (Rapireddy, et al., Biochemistry, 50(19):3913-8 (2011), He et al., “The Structure of a γ-modified peptide nucleic acid duplex”, Mol. BioSyst. 6:1619-1629 (2010); and Sahu et al., “Synthesis and Characterization of Conformationally Preorganized, (R)-Diethylene Glycol-Containing γ-Peptide Nucleic Acids with Superior Hybridization Properties and Water Solubility”, J. Org. Chem, 76:5614-5627) (2011)). Other advantageous properties can be conferred depending on the chemical nature of the specific substitution at the gamma position (the “R” group in the illustration of the Chiral γPNA, above).

One class of γ substitution, is miniPEG, but other residues and side chains can be considered, and even mixed substitutions can be used to tune the properties of the oligomers. “MiniPEG” and “MP” refers to diethylene glycol. MiniPEG-containing γPNAs are conformationally preorganized PNAs that exhibit superior hybridization properties and water solubility as compared to the original PNA design and other chiral γPNAs. Sahu et al., describes γPNAs prepared from L-amino acids that adopt a right-handed helix, and γPNAs prepared from D-amino acids that adopt a left-handed helix. Only the right-handed helical γPNAs hybridize to DNA or RNA with high affinity and sequence selectivity. In the most preferred embodiments, some or all of the PNA residues are miniPEG-containing γPNAs (Sahu, et al., J. Org. Chem., 76, 5614-5627 (2011). In some embodiments, tcPNAs are prepared wherein every other PNA residue on the Watson-Crick binding side of the linker is a miniPEG-containing γPNA. Accordingly, for these embodiments, the tail clamp side of the PNA has alternating classic PNA and miniPEG-containing γPNA residues.

In some embodiments PNA-mediated gene editing are achieved via additional or alternative γ substitutions or other PNA chemical modifications including but limited to those introduced above and below. Examples of γ substitution with other side chains include that of alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. The “derivatives thereof” herein are defined as those chemical moieties that are covalently attached to these amino acid side chains, for instance, to that of serine, cysteine, threonine, tyrosine, aspartic acid, glutamic acid, asparagine, glutamine, lysine, and arginine.

In addition to γPNAs showing consistently improved gene editing potency the level of off-target effects in the genome remains extremely low. This is in keeping with the lack of any intrinsic nuclease activity in the PNAs (in contrast to ZFNs or CRISPR/Cas9 or TALENS), and reflects the mechanism of triplex-induced gene editing, which acts by creating an altered helix at the target-binding site that engages endogenous high fidelity DNA repair pathways. As discussed above, the SCF/c-Kit pathway also stimulates these same pathways, providing for enhanced gene editing without increasing off-target risk or cellular toxicity.

Additionally, any of the triplex forming sequences can be modified to include guanidine-G-clamp (“G-clamp”) PNA residues(s) to enhance PNA binding, wherein the G-clamp is linked to the backbone as any other nucleobase would be. γPNAs with substitution of cytosine by clamp-G (9-(2-guanidinoethoxy) phenoxazine), a cytosine analog that can form five H-bonds with guanine, and can also provide extra base stacking due to the expanded phenoxazine ring system and substantially increased binding affinity. In vitro studies indicate that a single clamp-G substitution for C can substantially enhance the binding of a PNA-DNA duplex by 23° C. (Kuhn, et al., Artificial DNA, PNA & XNA, 1(1):45-53(2010)). As a result, γPNAs containing G-clamp substitutions can have further increased activity.

The structure of a clamp-G monomer-to-G base pair (clamp-G indicated by the “X”) is illustrated below in comparison to C-G base pair.

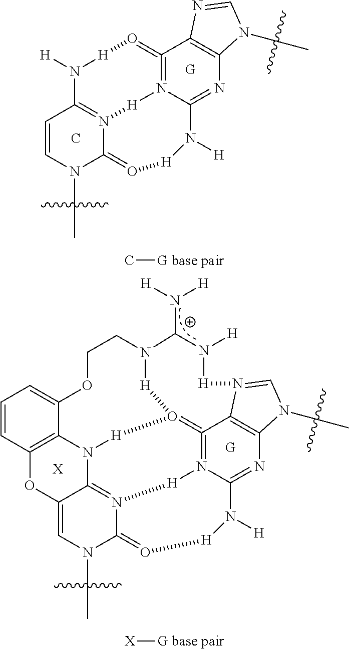

Some studies have shown improvements using D-amino acids in peptide synthesis.

In particular embodiments, the gene editing composition includes at least one peptide nucleic acid (PNA) oligomer. The at least one PNA oligomer can be a modified PNA oligomer including at least one modification at a gamma position of a backbone carbon. The modified PNA oligomer can include at least one miniPEG modification at a gamma position of a backbone carbon. The gene editing composition can include at least one donor oligonucleotide. The gene editing composition can modify a target sequence within a fetal genome.

The PNA can include a Hoogsteen binding peptide nucleic acid (PNA) segment and a Watson-Crick binding PNA segment collectively totaling no more than 50 nucleobases in length, wherein the two segments bind or hybridize to a target region of a genomic DNA comprising a polypurine stretch to induce strand invasion, displacement, and formation of a triple-stranded composition among the two PNA segments and the polypurine stretch of the genomic DNA, wherein the Hoogsteen binding segment binds to the target region by Hoogsteen binding for a length of least five nucleobases, and wherein the Watson-Crick binding segment binds to the target region by Watson-Crick binding for a length of least five nucleobases.

The PNA segments can include a gamma modification of a backbone carbon. The gamma modification can be a gamma miniPEG modification. The Hoogsteen binding segment can include one or more chemically modified cytosines selected from the group consisting of pseudocytosine, pseudoisocytosine, and 5-methylcytosine. The Watson-Crick binding segment can include a sequence of up to fifteen nucleobases that binds to the target duplex by Watson-Crick binding outside of the triplex. The two segments can be linked by a linker. In some embodiments, all of the peptide nucleic acid residues in the Hoogsteen-binding segment only, in the Watson-Crick-binding segment only, or across the entire PNA oligomer include a gamma modification of a backbone carbon. In some embodiments, one or more of the peptide nucleic acid residues in the Hoogsteen-binding segment only or in the Watson-Crick-binding segment only of the PNA oligomer include a gamma modification of a backbone carbon. In some embodiments, alternating peptide nucleic acid residues in the Hoogsteen-binding portion only, in the Watson-Crick-binding portion only, or across the entire PNA oligomer include a gamma modification of a backbone carbon.

In some embodiments, least one gamma modification of the backbone carbon is a gamma miniPEG modification. In some embodiments, at least one gamma modification is a side chain of an amino acid selected from the group consisting of alanine, serine, threonine, cysteine, valine, leucine, isoleucine, methionine, phenylalanine, tyrosine, aspartic acid, glutamic acid, asparagine, glutamine, histidine, lysine, arginine, and the derivatives thereof. In some embodiments, all gamma modifications are gamma miniPEG modifications. Optionally, at least one PNA segment comprises a clamp-G (9-(2-guanidinoethoxy) phenoxazine).

### 2. Triplex-Forming Target Sequence Considerations

The triplex-forming molecules bind to a predetermined target region referred to herein as the “target sequence,” “target region,” or “target site.” The target sequence for the triplex-forming molecules can be within or adjacent to a human gene encoding, for example the beta globin, cystic fibrosis transmembrane conductance regulator (CFTR) or other gene discussed in more detail below, or an enzyme necessary for the metabolism of lipids, glycoproteins, or mucopolysaccharides, or another gene in need of correction. The target sequence can be within the coding DNA sequence of the gene or within an intron. The target sequence can also be within DNA sequences which regulate expression of the target gene, including promoter or enhancer sequences or sites that regulate RNA splicing.

The nucleotide sequences of the triplex-forming molecules are selected based on the sequence of the target sequence, the physical constraints, and the preference for a low dissociation constant (K_{d}) for the triplex-forming molecules/target sequence. As used herein, triplex-forming molecules are said to be substantially complementary to a target region when the triplex-forming molecules has a nucleobase composition which allows for the formation of a triple-helix with the target region. A triplex-forming molecule can be substantially complementary to a target region even when there are non-complementary nucleobases present in the triplex-forming molecules.

There are a variety of structural motifs available which can be used to determine the nucleotide sequence of a substantially complementary oligonucleotide. Preferably, the triplex-forming molecules bind to or hybridize to the target sequence under conditions of high stringency and specificity. Reaction conditions for in vitro triple helix formation of an triplex-forming molecules probe or primer to a nucleic acid sequence vary from triplex-forming molecules to triplex-forming molecules, depending on factors such as the length triplex-forming molecules, the number of G:C and A:T base pairs, and the composition of the buffer utilized in the hybridization reaction.

a. Target Sequence Considerations for TFOs

Preferably, the TFO is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful. Most preferably, the oligonucleotides bind in a sequence-specific manner within the major groove of duplex DNA. An oligonucleotide substantially complementary, based on the third strand binding code, to the target region of the double-stranded nucleic acid molecule is preferred. The oligonucleotides will have a base composition which is conducive to triple-helix formation and will be generated based on one of the known structural motifs for third strand binding. The most stable complexes are formed on polypurine:polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C⁺.G:C and T.A:T. The triplex structures are stabilized by two Hoogsteen hydrogen bonds between the bases in the TFO strand and the purine strand in the duplex. A review of base compositions for third strand binding oligonucleotides is provided in U.S. Pat. No. 5,422,251.

TFOs are preferably generated using known DNA and/or PNA synthesis procedures. In one embodiment, oligonucleotides are generated synthetically. Oligonucleotides can also be chemically modified using standard methods that are well known in the art.

b. Target Sequence Considerations for PNAs

Some triplex-forming molecules, such as PNA, PNA clamps and tail clamp PNAs (tcPNAs) invade the target duplex, with displacement of the polypyrimidine strand, and induce triplex formation with the polypurine strand of the target duplex by both Watson-Crick and Hoogsteen binding. Preferably, both the Watson-Crick and Hoogsteen binding portions of the triplex forming molecules are substantially complementary to the target sequence. Although, as with triplex-forming oligonucleotides, a homopurine strand is needed to allow formation of a stable PNA/DNA/PNA triplex, PNA clamps can form at shorter homopurine sequences than those required by triplex-forming oligonucleotides and also do so with greater stability.

Preferably, PNAs are between 6 and 50 nucleobase-containing residues in length. The Watson-Crick portion should be 9 or more nucleobase-containing residues in length, optionally including a tail sequence. More preferably, the Watson-Crick binding portion is between about 9 and 30 nucleobase-containing residues in length, optionally including a tail sequence of between 0 and about 15 nucleobase-containing residues. More preferably, the Watson-Crick binding portion is between about 10 and 25 nucleobase-containing residues in length, optionally including a tail sequence of between 0 and about 10 nucleobase-containing residues in length. In the most preferred embodiment, the Watson-Crick binding portion is between 15 and 25 nucleobase-containing residues in length, optionally including a tail sequence of between 5 and 10 nucleobase-containing residues in length. The Hoogsteen binding portion should be 6 or more nucleobase residues in length. Most preferably, the Hoogsteen binding portion is between about 6 and 15 nucleobase-containing residues in length, inclusive.

The triplex-forming molecules are designed to target the polypurine strand of a polypurine:polypyrimidine stretch in the target duplex nucleotide. Therefore, the base composition of the triplex-forming molecules may be homopyrimidine. Alternatively, the base composition may be polypyrimidine. The addition of a “tail” reduces the requirement for polypurine:polypyrimidine run. Adding additional nucleobase-containing residues, known as a “tail,” to the Watson-Crick binding portion of the triplex-forming molecules allows the Watson-Crick binding portion to bind/hybridize to the target strand outside the site of polypurine sequence for triplex formation. These additional bases further reduce the requirement for the polypurine:polypyrimidine stretch in the target duplex and therefore increase the number of potential target sites. Triplex-forming molecules (TFMs) including, e.g., triplex-forming oligonucleotides (TFOs) and helix-invading peptide nucleic acids (bis-PNAs and tcPNAs), also generally utilize a polypurine:polypyrimidine sequence to a form a triple helix. Traditional nucleic acid TFOs may need a stretch of at least 15 and preferably 30 or more nucleobase-containing residues. Peptide nucleic acids need fewer purines to a form a triple helix, although at least 10 or preferably more may be needed. Peptide nucleic acids including a tail, also referred to tail clamp PNAs, or tcPNAs, require even fewer purines to a form a triple helix. A triple helix may be formed with a target sequence containing fewer than 8 purines. Therefore, PNAs should be designed to target a site on duplex nucleic acid containing between 6-30 polypurine:polypyrimidines, preferably, 6-25 polypurine:polypyrimidines, more preferably 6-20 polypurine:polypyrimidines.

The addition of a “mixed-sequence” tail to the Watson-Crick-binding strand of the triplex-forming molecules such as PNAs also increases the length of the triplex-forming molecule and, correspondingly, the length of the binding site. This increases the target specificity and size of the lesion created at the target site and disrupts the helix in the duplex nucleic acid, while maintaining a low requirement for a stretch of polypurine:polypyrimidines. Increasing the length of the target sequence improves specificity for the target, for example, a target of 17 base pairs will statistically be unique in the human genome. Relative to a smaller lesion, it is likely that a larger triplex lesion with greater disruption of the underlying DNA duplex will be detected and processed more quickly and efficiently by the endogenous DNA repair machinery that facilitates recombination of the donor oligonucleotide.

The triple-forming molecules are preferably generated using known synthesis procedures. In one embodiment, triplex-forming molecules are generated synthetically. Triplex-forming molecules can also be chemically modified using standard methods that are well known in the art.

### B. Pseudocomplementary Oligonucleotides/PNAs

The gene editing technology can be pseudocomplementary oligonucleotides such as those disclosed in U.S. Pat. No. 8,309,356. “Double duplex-forming molecules,” are oligonucleotides that bind to duplex DNA in a sequence-specific manner to form a four-stranded structure. Double duplex-forming molecules, such as a pair of pseudocomplementary oligonucleotides/PNAs, can induce recombination with a donor oligonucleotide at a chromosomal site in mammalian cells. Pseudocomplementary oligonucleotides/PNAs are complementary oligonucleotides/PNAs that contain one or more modifications such that they do not recognize or hybridize to each other, for example due to steric hindrance, but each can recognize and hybridize to its complementary nucleic acid strands at the target site. As used herein the term ‘pseudocomplementary oligonucleotide(s)’ include pseudocomplementary peptide nucleic acids (pcPNAs). A pseudocomplementary oligonucleotide is said to be substantially complementary to a target region when the oligonucleotide has a base composition which allows for the formation of a double duplex with the target region. As such, an oligonucleotide can be substantially complementary to a target region even when there are non-complementary bases present in the pseudocomplementary oligonucleotide.

This strategy can be more efficient and provides increased flexibility over other methods of induced recombination such as triple-helix oligonucleotides and bis-peptide nucleic acids which prefer a polypurine sequence in the target double-stranded DNA. The design ensures that the pseudocomplementary oligonucleotides do not pair with each other but instead bind the cognate nucleic acids at the target site, inducing the formation of a double duplex.

The predetermined region that the double duplex-forming molecules bind to can be referred to as a “double duplex target sequence,” “double duplex target region,” or “double duplex target site.” The double duplex target sequence (DDTS) for the double duplex-forming molecules can be, for example, within or adjacent to a human gene in need of induced gene correction. The DDTS can be within the coding DNA sequence of the gene or within introns. The DDTS can also be within DNA sequences which regulate expression of the target gene, including promoter or enhancer sequences.

The nucleotide/nucleobase sequence of the pseudocomplementary oligonucleotides is selected based on the sequence of the DDTS. Therapeutic administration of pseudocomplementary oligonucleotides involves two single stranded oligonucleotides unlinked, or linked by a linker. One pseudocomplementary oligonucleotide strand is complementary to the DDTS, while the other is complementary to the displaced DNA strand. The use of pseudocomplementary oligonucleotides, particularly pcPNAs are not subject to limitation on sequence choice and/or target length and specificity as are triplex-forming oligonucleotides, helix-invading peptide nucleic acids (bis-PNAs and tcPNAs) and side-by-side minor groove binders. Pseudocomplementary oligonucleotides do not require third-strand Hoogsteen-binding, and therefore are not restricted to homopurine targets. Pseudocomplementary oligonucleotides can be designed for mixed, general sequence recognition of a desired target site. Preferably, the target site contains an A:T base pair content of about 40% or greater. Preferably pseudocomplementary oligonucleotides are between about 8 and 50 nucleobase-containing residues in length, more preferably 8 to 30, even more preferably between about 8 and 20 nucleobase-containing residues in length.

The pseudocomplementary oligonucleotides should be designed to bind to the target site (DDTS) at a distance of between about 1 to 800 bases from the target site of the donor oligonucleotide. More preferably, the pseudocomplementary oligonucleotides bind at a distance of between about 25 and 75 bases from the donor oligonucleotide. Most preferably, the pseudocomplementary oligonucleotides bind at a distance of about 50 bases from the donor oligonucleotide. Preferred pcPNA sequences for targeted repair of a mutation in the β-globin intron IVS2 (G to A) are described in U.S. Pat. No. 8,309,356.

Preferably, the pseudocomplementary oligonucleotides bind/hybridize to the target nucleic acid molecule under conditions of high stringency and specificity. Most preferably, the oligonucleotides bind in a sequence-specific manner and induce the formation of double duplex. Specificity and binding affinity of the pseudocomplemetary oligonucleotides may vary from oligomer to oligomer, depending on factors such as length, the number of G:C and A:T base pairs, and the formulation.

### C. CRISPR/Cas

In some embodiments, the gene editing composition is the CRISPR/Cas system. CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with the required elements including a cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423, which are specifically incorporated by reference herein in their entireties.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some embodiments, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within a sgRNA, the crRNA portion can be identified as the “target sequence” and the tracrRNA is often referred to as the “scaffold.”

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequences.

In some embodiments, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence (such as CTPS1) can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligomers that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

### D. Zinc Finger Nucleases

In some embodiments, the element that induces a single or a double strand break in the target cell's genome is a nucleic acid construct or constructs encoding a zinc finger nucleases (ZFNs). ZFNs are typically fusion proteins that include a DNA-binding domain derived from a zinc-finger protein linked to a cleavage domain.

The most common cleavage domain is the Type IIS enzyme Fokl. Fok1 catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. Proc., Natl. Acad. Sci. USA 89 (1992):4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31,978-31,982 (1994b). One or more of these enzymes (or enzymatically functional fragments thereof) can be used as a source of cleavage domains.

The DNA-binding domain, which can, in principle, be designed to target any genomic location of interest, can be a tandem array of Cys₂His₂ zinc fingers, each of which generally recognizes three to four nucleotides in the target DNA sequence. The Cys₂His₂ domain has a general structure: Phe (sometimes Tyr)-Cys-(2 to 4 amino acids)-Cys-(3 amino acids)-Phe (sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long.

Engineering methods include, but are not limited to, rational design and various types of empirical selection methods. Rational design includes, for example, using databases including triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,067,617; U.S. Published Application Nos. 2002/0165356; 2004/0197892; 2007/0154989; 2007/0213269; and International Patent Application Publication Nos. WO 98/53059 and WO 2003/016496.

### E. Transcription Activator-Like Effector Nucleases

In some embodiments, the element that induces a single or a double strand break in the target cell's genome is a nucleic acid construct or constructs encoding a transcription activator-like effector nuclease (TALEN). TALENs have an overall architecture similar to that of ZFNs, with the main difference that the DNA-binding domain comes from TAL effector proteins, transcription factors from plant pathogenic bacteria. The DNA-binding domain of a TALEN is a tandem array of amino acid repeats, each about 34 residues long. The repeats are very similar to each other; typically they differ principally at two positions (amino acids 12 and 13, called the repeat variable diresidue, or RVD). Each RVD specifies preferential binding to one of the four possible nucleotides, meaning that each TALEN repeat binds to a single base pair, though the NN RVD is known to bind adenines in addition to guanine. TAL effector DNA binding is mechanistically less well understood than that of zinc-finger proteins, but their seemingly simpler code could prove very beneficial for engineered-nuclease design. TALENs also cleave as dimers, have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats.

Methods of engineering TAL to bind to specific nucleic acids are described in Cermak, et al, Nucl. Acids Res. 1-11 (2011). U.S. Published Application No. 2011/0145940, which discloses TAL effectors and methods of using them to modify DNA. Miller et al. Nature Biotechnol 29: 143 (2011) reported making TALENs for site-specific nuclease architecture by linking TAL truncation variants to the catalytic domain of Fokl nuclease. The resulting TALENs were shown to induce gene modification in immortalized human cells. General design principles for TALE binding domains can be found in, for example, WO 2011/072246.

### V. Donor Oligonucleotides

In some embodiments, the gene editing composition includes or is administered in combination with a donor oligonucleotide. The donor oligonucleotide can be encapsulated or entrapped in the same or different particles from other active agents such as the triplex forming composition. Generally, in the case of gene therapy, the donor oligonucleotide includes a sequence that can correct a mutation(s) in the host genome, though in some embodiments, the donor introduces a mutation that can, for example, reduce expression of an oncogene or a receptor that facilitates HIV infection. In addition to containing a sequence designed to introduce the desired correction or mutation, the donor oligonucleotide may also contain synonymous (silent) mutations (e.g., 7 to 10). The additional silent mutations can facilitate detection of the corrected target sequence using allele-specific PCR of genomic DNA isolated from treated cells. Triplex-forming composition and other gene editing compositions such as those discussed above can increase the rate of recombination of the donor oligonucleotide in the target cells relative to administering donor alone.

### A. Preferred Donor Oligonucleotide Design for Triplex and Double-Duplex Based Technologies

The triplex forming molecules including peptide nucleic acids may be administered in combination with, or tethered to, a donor oligonucleotide via a mixed sequence linker or used in conjunction with a non-tethered donor oligonucleotide that is substantially homologous to the target sequence. Triplex-forming molecules can induce recombination of a donor oligonucleotide sequence up to several hundred base pairs away. It is preferred that the donor oligonucleotide sequence targets a region between 0 to 800 bases from the target binding site of the triplex-forming molecules. More preferably the donor oligonucleotide sequence targets a region between 25 to 75 bases from the target binding site of the triplex-forming molecules. Most preferably that the donor oligonucleotide sequence targets a region about 50 nucleotides from the target binding site of the triplex-forming molecules.

The donor sequence can contain one or more nucleic acid sequence alterations compared to the sequence of the region targeted for recombination, for example, a substitution, a deletion, or an insertion of one or more nucleotides. Successful recombination of the donor sequence results in a change of the sequence of the target region. Donor oligonucleotides are also referred to herein as donor fragments, donor nucleic acids, donor DNA, or donor DNA fragments. This strategy exploits the ability of a triplex to provoke DNA repair, potentially increasing the probability of recombination with the homologous donor DNA. It is understood in the art that a greater number of homologous positions within the donor fragment will increase the probability that the donor fragment will be recombined into the target sequence, target region, or target site. Tethering of a donor oligonucleotide to a triplex-forming molecule facilitates target site recognition via triple helix formation while at the same time positioning the tethered donor fragment for possible recombination and information transfer. Triplex-forming molecules also effectively induce homologous recombination of non-tethered donor oligonucleotides. The term “recombinagenic” as used herein, is used to define a DNA fragment, oligonucleotide, peptide nucleic acid, or composition as being able to recombine into a target site or sequence or induce recombination of another DNA fragment, oligonucleotide, or composition.

Non-tethered or unlinked fragments may range in length from 20 nucleotides to several thousand. The donor oligonucleotide molecules, whether linked or unlinked, can exist in single stranded or double stranded form. The donor fragment to be recombined can be linked or un-linked to the triplex forming molecules. The linked donor fragment may range in length from 4 nucleotides to 100 nucleotides, preferably from 4 to 80 nucleotides in length. However, the unlinked donor fragments have a much broader range, from 20 nucleotides to several thousand. In one embodiment the oligonucleotide donor is between 25 and 80 nucleobases. In a further embodiment, the non-tethered donor oligonucleotide is about 50 to 60 nucleotides in length.

The donor oligonucleotides contain at least one mutated, inserted or deleted nucleotide relative to the target DNA sequence. Target sequences can be within the coding DNA sequence of the gene or within introns. Target sequences can also be within DNA sequences which regulate expression of the target gene, including promoter or enhancer sequences or sequences that regulate RNA splicing.

The donor oligonucleotides can contain a variety of mutations relative to the target sequence. Representative types of mutations include, but are not limited to, point mutations, deletions and insertions. Deletions and insertions can result in frameshift mutations or deletions. Point mutations can cause missense or nonsense mutations. These mutations may disrupt, reduce, stop, increase, improve, or otherwise alter the expression of the target gene.

Compositions including triplex-forming molecules such as tcPNA may include one or more than one donor oligonucleotides. More than one donor oligonucleotides may be administered with triplex-forming molecules in a single transfection, or sequential transfections. Use of more than one donor oligonucleotide may be useful, for example, to create a heterozygous target gene where the two alleles contain different modifications.

Donor oligonucleotides are preferably DNA oligonucleotides, composed of the principal naturally-occurring nucleotides (uracil, thymine, cytosine, adenine and guanine) as the heterocyclic nucleobases, deoxyribose as the sugar moiety, and phosphate ester linkages. Donor oligonucleotides may include modifications to nucleobases, sugar moieties, or backbone/linkages, as described above, depending on the desired structure of the replacement sequence at the site of recombination or to provide some resistance to degradation by nucleases. One exemplary modification is a thiophosphate ester linkage. Modifications to the donor oligonucleotide should not prevent the donor oligonucleotide from successfully recombining at the recombination target sequence in the presence of triplex-forming molecules.

### B. Preferred Donor Oligonucleotides Design for Nuclease-Based Technologies

The nuclease activity of the genome editing systems described herein cleave target DNA to produce single or double strand breaks in the target DNA. Double strand breaks can be repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site.

Therefore, in some embodiments, the genome editing composition optionally includes a donor oligonucleotide. The modifications of the target DNA due to NHEJ and/or homology-directed repair can be used to induce gene correction, gene replacement, gene tagging, transgene insertion, nucleotide deletion, gene disruption, gene mutation, etc.

Accordingly, cleavage of DNA by the genome editing composition can be used to delete nucleic acid material from a target DNA sequence by cleaving the target DNA sequence and allowing the cell to repair the sequence in the absence of an exogenously provided donor polynucleotide. Alternatively, if the genome editing composition includes a donor oligonucleotide sequence that includes at least a segment with homology to the target DNA sequence, the methods can be used to add, i.e., insert or replace, nucleic acid material to a target DNA sequence (e.g., to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, the compositions can be used to modify DNA in a site-specific, i.e., “targeted”, way, for example gene knock-out, gene knock-in, gene editing, gene tagging, etc. as used in, for example, gene therapy.

In applications in which it is desirable to insert an oligonucleotide sequence into a target DNA sequence, an oligonucleotide including a donor sequence to be inserted is also provided to the cell. By a “donor sequence” or “donor polynucleotide” or “donor oligonucleotide” it is meant a nucleic acid sequence to be inserted at the cleavage site. The donor polynucleotide typically contains sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor oligonucleotide includes a non-homologous sequence flanked by two regions of homology, such that homology-directed repair between the target DNA region and the two flanking sequences results in insertion of the non-homologous sequence at the target region.

### VI. Nucleobase, Sugar, and Linkage Modifications

Any of the gene editing technologies, components thereof, donor oligonucleotides, or other nucleic acids disclosed herein can include one or more modifications or substitutions to the nucleobases or linkages. Although modifications are particularly preferred for use with triplex-forming technologies and typically discussed below with reference thereto, any of the modifications can be utilized in the construction of any of the gene editing compositions, donor, nucleotides, etc. Modifications should not prevent, and preferably enhance the activity, persistence, or function of the gene editing technology. For example, modifications to oligonucleotides for use as triplex-forming molecules should not prevent, and preferably enhance duplex invasion, strand displacement, and/or stabilize triplex formation as described above by increasing specificity or binding affinity of the triplex-forming molecules to the target site. Modified bases and base analogues, modified sugars and sugar analogues and/or various suitable linkages known in the art are also suitable for use in the molecules disclosed herein. Several preferred oligonucleotide compositions including PNA, and modification thereof to include MiniPEG at the γ position in the PNA backbone, are discussed above. Additional modifications are discussed in more detail below.

### A. Nucleobases

The principal naturally-occurring nucleotides include uracil, thymine, cytosine, adenine and guanine as the heterocyclic nucleobases. Gene editing molecules can include chemical modifications to their nucleotide constituents. For example, target sequences with adjacent cytosines can be problematic. Triplex stability is greatly compromised by runs of cytosines, thought to be due to repulsion between the positive charge resulting from the N³ protonation or perhaps because of competition for protons by the adjacent cytosines. Chemical modification of nucleotides including triplex-forming molecules such as PNAs may be useful to increase binding affinity of triplex-forming molecules and/or triplex stability under physiologic conditions.

Chemical modifications of nucleobases or nucleobase analogs may be effective to increase the binding affinity of a nucleotide or its stability in a triplex. Chemically-modified nucleobases include, but are not limited to, inosine, 5-(1-propynyl) uracil (pU), 2-thio uracil, 5-(1-propynyl) cytosine (pC), 5-methylcytosine, 8-oxo-adenine, 2,6-diaminopurine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives. Substitution of 5-methylcytosine or pseudoisocytosine for cytosine in triplex-forming molecules such as PNAs helps to stabilize triplex formation at neutral and/or physiological pH, especially in triplex-forming molecules with isolated cytosines.

### B. Backbone

The nucleotide residues of the triplex-forming molecules such as PNAs are connected by an internucleotide bond that refers to a chemical linkage between two nucleoside moieties. Unmodified peptide nucleic acids (PNAs) are synthetic DNA mimics in which the phosphate backbone of the oligonucleotide is replaced in its entirety by repeating N-(2-aminoethyl)-glycine units that are linked by amide bonds. The various nucleobases are linked to the backbone by methylene carbonyl bonds, which allow them to form PNA-DNA or PNA-RNA duplexes via Watson-Crick base pairing with high affinity and sequence-specificity. PNAs maintain spacing of nucleobases that is similar to conventional DNA oligonucleotides, but are achiral and neutrally charged molecules. Peptide nucleic acids are composed of peptide nucleic acid residues.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine are particularly useful if positive charges are desired in the PNA, and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

Backbone modifications used to generate triplex-forming molecules should not prevent the molecules from binding with high specificity to the target site and creating a triplex with the target duplex nucleic acid by displacing one strand of the target duplex and forming a clamp around the other strand of the target duplex.

### C. Modified Nucleic Acids

Modified nucleic acids in addition to peptide nucleic acids are also useful as triplex-forming molecules. Oligonucleotides are composed a chain of nucleotides which are linked to one another. Canonical nucleotides typically are composed of a nucleobase (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic nucleobases, and ribose or deoxyribose sugar linked by phosphodiester bonds. As used herein “modified nucleotide” or “chemically modified nucleotide” defines a nucleotide that has a chemical modification of one or more of the nucleobase, sugar moiety or phosphate moiety constituents. Preferably the charge of the modified nucleotide is reduced compared to DNA or RNA oligonucleotides of the same nucleobase sequence. Most preferably the triplex-forming molecules have low negative charge, no charge, or positive charge such that electrostatic repulsion with the nucleotide duplex at the target site is reduced compared to DNA or RNA oligonucleotides with the corresponding nucleobase sequence.

Examples of modified nucleotides with reduced charge include modified internucleotide linkages such as phosphate analogs having achiral and uncharged intersubunit linkages (e.g., Sterchak, E. P. et al., Organic Chem., 52:4202, (1987)), and uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be except they have a negatively charged backbone, whereas PNAs generally have a neutrally charged backbone (although certain amino acid side chain modifications can alter the backbone charge). LNA binding efficiency can be increased in some embodiments by adding positive charges to it. Commercial nucleic acid synthesizers and standard phosphoramidite chemistry can be used to make LNAs.

Molecules may also include nucleotides with modified nucleobases, sugar moieties or sugar moiety analogs. Modified nucleotides may include modified nucleobases or base analogs as described above with respect to peptide nucleic acids. Sugar moiety modifications include, but are not limited to, 2′-O-aminoethoxy, 2′-O-amonioethyl (2′-OAE), 2′-O-methoxy, 2′-O-methyl, 2-guanidoethyl (2′-OGE), 2′-0,4′-C-methylene (LNA), 2′-O-(methoxyethyl) (2′-OME) and 2′-O—(N-(methyl)acetamido) (2′-OMA). 2′-O-aminoethyl sugar moiety substitutions are especially preferred because they are protonated at neutral pH and thus suppress the charge repulsion between the triplex-forming molecule and the target duplex. This modification stabilizes the C3′-endo conformation of the ribose or deoxyribose and also forms a bridge with the i-1 phosphate in the purine strand of the duplex.

### VII. Gene Editing Potentiating Factors

In some embodiments, the compositions and methods include a potentiating factor. For example, certain potentiating factors can be used to increase the efficacy of gene editing technologies. Gene expression profiling on SCF-treated CD117+ cells versus untreated CD117+ cells discussed in the Examples below showed additional up-regulation of numerous DNA repair genes including RAD51 and BRCA2. These results and others discussed below indicate that a functional c-Kit signaling pathway mediates increased HDR and promotes gene editing, rather than CD117 simply being a phenotypic marker. When CD117+ cells were treated with SCF, expression of these DNA repair genes was increased even more, correlating with a further increase in gene editing.

Accordingly, compositions and methods of increasing the efficacy of gene editing technology are provided. As used herein a “gene editing potentiating factor” or “gene editing potentiating agent” or “potentiating factor or “potentiating agent” refers a compound that increases the efficacy of editing (e.g., mutation, including insertion, deletion, substitution, etc.) of a gene, genome, or other nucleic acid) by a gene editing technology relative to use of the gene editing technology in the absence of the compound. Preferred gene editing technologies suitable for use alone or more preferably in combination with the potentiating factors are discussed in more detail below. In some embodiments, the potentiating factor is administered as a nucleic acid encoding the potentiating factor. In certain preferred embodiments, the gene editing technology is a triplex-forming γPNA and donor DNA, optionally, but preferably in a particle composition.

Potentiating factors include, for example, DNA damage or repair-stimulating or -potentiating factors. Preferably the factor is one that engages one or more endogenous high fidelity DNA repair pathways. In some embodiments, the factor is one that increases expression of Rad51, BRCA2, or a combination thereof.

As discussed in more detail below, the preferred methods typically include contacting cells with an effective amount of a gene editing potentiating factor. The contacting can occur ex vivo, for example isolated cells, or in vivo following, for example, administration of the potentiating factor to a subject. Exemplary gene editing potentiating agents include receptor tyrosine kinase C-kit ligands, ATR-Chk1 cell cycle checkpoint pathway inhibitors, a DNA polymerase alpha inhibitors, and heat shock protein 90 inhibitors (HSP90i).

### A. C-Kit Ligands

In some embodiments, the factor is an activator of the receptor tyrosine kinase c-Kit. CD117 (also known as mast/stem cell growth factor receptor or proto-oncogene c-Kit protein) is a receptor tyrosine kinase expressed on the surface of hematopoietic stem and progenitor cells as well as other cell types. Stem cell factor (SCF), the ligand for c-Kit, causes dimerization of the receptor and activates its tyrosine kinase activity to trigger downstream signaling pathways that can impact survival, proliferation, and differentiation. SCF and c-Kit are reviewed in Lennartsson and Rönnstrand, Physiological Reviews, 92(4):1619-1649 (2012)). Thus, in some embodiments, the C-kit ligand is stem factor protein or fragment thereof sufficient to causes dimerization of C-kit and activates its tyrosine kinase activity. The C-kit ligand can be a nucleic acid encoding a stem factor protein or fragment thereof sufficient to causes dimerization of C-kit and activates its tyrosine kinase activity. The nucleic acid can be an mRNA or an expression vector.

The human SCF gene encodes for a 273 amino acid transmembrane protein, which contains a 25 amino acid N-terminal signal sequence, a 189 amino acid extracellular domain, a 23 amino acid transmembrane domain, and a 36 amino acid cytoplasmic domain.

The secreted soluble form of SCF is generated by proteolytic processing of the membrane-anchored precursor.

Murine and rat SCF are fully active on human cells.

It will be appreciated that SCF can be administered to cells or a subject as SCF protein, or as a nucleic acid encoding SCF (transcribed RNA, DNA, DNA in an expression vector). Accordingly, nucleic acid sequences, including RNA (e.g., mRNA) and DNA sequences are also provided, both alone and inserted into expression cassettes and vectors. For example, a sequence encoding SCF can be incorporated into an autonomously replicating plasmid, a virus (e.g., a retrovirus, lentivirus, adenovirus, or herpes virus), or into the genomic DNA of a prokaryote or eukaryote.

The observed effect of SCF indicates that other cytokines or growth factors including, but not limited to, erythropoietin, GM-CSF, EGF (especially for epithelial cells; lung epithelia for cystic fibrosis), hepatocyte growth factor etc., could similarly serve to boost gene editing potential in bone marrow cells or in other tissues. In some embodiments, gene editing is enhanced in specific cell types using cytokines targeted to these cell types.

### B. Replication Modulators

In some embodiments, the potentiating factor is a replication modulator that can, for example, manipulate replication progression and/or replication forks. For example, the ATR-Chk1 cell cycle checkpoint pathway has numerous roles in protecting cells from DNA damage and stalled replication, one of the most prominent being control of the cell cycle and prevention of premature entry into mitosis (Thompson and Eastman, Br J Clin Pharmacol., 76(3): 358-369 (2013), Smith, et al., Adv Cancer Res., 108:73-112 (2010)). However, Chk1 also contributes to the stabilization of stalled replication forks, the control of replication origin firing and replication fork progression, and homologous recombination. DNA polymerase alpha also known as Pol a is an enzyme complex found in eukaryotes that is involved in initiation of DNA replication. Hsp90 (heat shock protein 90) is a chaperone protein that assists other proteins to fold properly, stabilizes proteins against heat stress, and aids in protein degradation.

Experimental results show that inhibitors of CHK1 and ATR in the DNA damage response pathway, as well as DNA polymerase alpha inhibitors and HSP90 inhibitors, substantially boost gene editing by triplex-forming PNAs and single-stranded donor DNA oligonucleotides. Accordingly, in some embodiments, the potentiating factor is a CHK1 or ATR pathway inhibitor, a DNA polymerase alpha inhibitor, or an HSP90 inhibitor. The inhibitor can be a functional nucleic acid, for example siRNA, miRNA, aptamers, ribozymes, triplex forming molecules, RNAi, or external guide sequences that targets CHK1, ATR, or another molecule in the ATR-Chk1 cell cycle checkpoint pathway; DNA polymerase alpha; or HSP90 and reduces expression or active of ATR, CHK1, DNA polymerase alpha, or HSP90.

Preferably, the inhibitor is a small molecule. For example, the potentiating factor can be a small molecule inhibitor of ATR-Chk1 Cell Cycle Checkpoint Pathway Inhibitor. Such inhibitors are known in the art, and many have been tested in clinical trials for the treatment of cancer. Exemplary CHK1 inhibitors include, but are not limited to, AZD7762, SCH900776/MK-8776, IC83/LY2603618, LY2606368, GDC-0425, PF-00477736, XL844, CEP-3891, SAR-020106, CCT-244747, Arry-575 (Thompson and Eastman, Br J Clin Pharmacol., 76(3): 358-369 (2013)), and SB218075. Exemplary ATR pathway inhibitors include, but are not limited to Schisandrin B, NU6027, NVP-BEZ235, VE-821, VE-822 (VX-970), AZ20, AZD6738, MIRIN, KU5593, VE-821, NU7441, LCA, and L189 (Weber and Ryan, Pharmacology & Therapeutics, 149:124-138 (2015)).

In some embodiments, the potentiating factor is a DNA polymerase alpha inhibitor, such as aphidicolin.

In some embodiments, the potentiating factor is a heat shock protein 90 inhibitor (HSP90i) such as STA-9090 (ganetespib). Other HSP90 inhibitors are known in the art and include, but are not limited to, benzoquinone ansamycin antibiotics such as geldanamycin (GA); 17-AAG (17-Allylamino-17-demethoxy-geldanamycin); 17-DMAG (17-dimethylaminoethylamino-17-demethoxy-geldanamycin) (Alvespimycin); IPI-504 (Retaspimycin); and AUY922 (Tatokoro, et al., EXCLI J., 14:48-58 (2015)).

### VIII. Functional Molecules

Functional molecules can be associated with, linked, conjugated, or otherwise attached directly or indirectly gene editing technology, potentiating agents, or particles utilized for delivery thereof. For example, the composition can include a targeting agent, a cell penetrating peptide, or a combination thereof. In some embodiments, two or more targeting molecules are used. The targeting agent is typically capable of specifically binding to a target on or in the fetus or embryo. Target agents can be bound or conjugated to particles (e.g., a polymer of the particle).

### A. Targeting Molecules

One class of functional elements is targeting molecules. Targeting molecules can be associated with, linked, conjugated, or otherwise attached directly or indirectly to the gene editing molecule, or to a particle or other delivery vehicle thereof.

Targeting molecules can be proteins, peptides, nucleic acid molecules, saccharides or polysaccharides that bind to a receptor or other molecule on the surface of a targeted cell. The degree of specificity and the avidity of binding to the graft can be modulated through the selection of the targeting molecule. For example, antibodies are very specific. These can be polyclonal, monoclonal, fragments, recombinant, or single chain, many of which are commercially available or readily obtained using standard techniques.

Examples of moieties include, for example, targeting moieties which provide for the delivery of molecules to specific cells, e.g., antibodies to hematopoietic stem cells, CD34⁺ cells, T cells or any other preferred cell type, as well as receptor and ligands expressed on the preferred cell type. Preferably, the moieties target hematopoeitic stem cells.

In some embodiments, the targeting molecule targets a cell surface protein.

Examples of molecules targeting extracellular matrix (“ECM”) include glycosaminoglycan (“GAG”) and collagen. In one embodiment, the external surface of polymer particles may be modified to enhance the ability of the particles to interact with selected cells or tissue. The method described above wherein an adaptor element conjugated to a targeting molecule is inserted into the particle is preferred. However, in another embodiment, the outer surface of a polymer micro- or nanoparticle having a carboxy terminus may be linked to targeting molecules that have a free amine terminus.

Other useful ligands attached to polymeric micro- and nanoparticles include pathogen-associated molecular patterns (PAMPs). PAMPs target Toll-like Receptors (TLRs) on the surface of the cells or tissue, or signal the cells or tissue internally, thereby potentially increasing uptake. PAMPs conjugated to the particle surface or co-encapsulated may include: unmethylated CpG DNA (bacterial), double-stranded RNA (viral), lipopolysacharride (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic) acid (bacterial), lipoteichoic acid (bacterial) or imidazoquinolines (synthetic).

In another embodiment, the outer surface of the particle may be treated using a mannose amine, thereby mannosylating the outer surface of the particle. This treatment may cause the particle to bind to the target cell or tissue at a mannose receptor on the antigen presenting cell surface. Alternatively, surface conjugation with an immunoglobulin molecule containing an Fc portion (targeting Fc receptor), heat shock protein moiety (HSP receptor), phosphatidylserine (scavenger receptors), and lipopolysaccharide (LPS) are additional receptor targets on cells or tissue.

Lectins that can be covalently attached to micro- and nanoparticles to render them target specific to the mucin and mucosal cell layer include lectins isolated from Abrus precatroius, Agaricus bisporus, Anguilla, Arachis hypogaea, Pandeiraea simplicifolia, Bauhinia purpurea, Caragan arobrescens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The choice of targeting molecule will depend on the method of administration of the particle composition and the cells or tissues to be targeted. The targeting molecule may generally increase the binding affinity of the particles for cell or tissues or may target the particle to a particular tissue in an organ or a particular cell type in a tissue. Avidin increases the ability of polymeric particles to bind to tissues. While the exact mechanism of the enhanced binding of avidin-coated particles to tissues has not been elucidated, it is hypothesized it is caused by electrostatic attraction of positively charged avidin to the negatively charged extracellular matrix of tissue. Non-specific binding of avidin, due to electrostatic interactions, has been previously documented and zeta potential measurements of avidin-coated PLGA particles revealed a positively charged surface as compared to uncoated PLGA particles.

The attachment of any positively charged ligand, such as polyethyleneimine or polylysine, to any polymeric particle may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most particles with the appropriate chemistry and be expected to influence the binding of particles to the gut. For example, polyclonal antibodies raised against components of mucin or else intact mucin, when covalently coupled to particles, would provide for increased bioadhesion. Similarly, antibodies directed against specific cell surface receptors exposed on the lumenal surface of the intestinal tract would increase the residence time of beads, when coupled to particles using the appropriate chemistry. The ligand affinity need not be based only on electrostatic charge, but other useful physical parameters such as solubility in mucin or else specific affinity to carbohydrate groups.

The covalent attachment of any of the natural components of mucin in either pure or partially purified form to the particles would decrease the surface tension of the bead-gut interface and increase the solubility of the bead in the mucin layer. The list of useful ligands includes, but is not limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, e.g., polyaspartic acid and polyglutamic acid, should also provide a useful means of increasing bioadhesiveness. Using polyamino acids in the 15,000 to 50,000 kDa molecular weight range yields chains of 120 to 425 amino acid residues attached to the surface of the particles. The polyamino chains increase bioadhesion by means of chain entanglement in mucin strands as well as by increased carboxylic charge.

The efficacy of the particles is determined in part by their route of administration into the body. For orally and topically administered particles, epithelial cells constitute the principal barrier that separates an organism's interior from the outside world. Epithelial cells such as those that line the gastrointestinal tract form continuous monolayers that simultaneously confront the extracellular fluid compartment and the extracorporeal space.

Adherence to cells is an essential first step in crossing the epithelial barrier by any of these mechanisms. Therefore, in one embodiment, the particles herein further include epithelial cell targeting molecules. Epithelial cell targeting molecules include monoclonal or polyclonal antibodies or bioactive fragments thereof that recognize and bind to epitopes displayed on the surface of epithelial cells. Epithelial cell targeting molecules also include ligands which bind to a cell surface receptor on epithelial cells. Ligands include, but are not limited to, molecules such as polypeptides, nucleotides and polysaccharides.

A variety of receptors on epithelial cells may be targeted by epithelial cell targeting molecules. Examples of suitable receptors to be targeted include, but are not limited to, IgE Fc receptors, EpCAM, selected carbohydrate specificites, dipeptidyl peptidase, and E-cadherin.

### B. Protein Transduction Domains and Fusogenic Peptides

Other functional elements that can be associated with, linked, conjugated, or otherwise attached directly or indirectly to the gene editing molecule, potentiating agent, or to a particle or other delivery vehicle thereof, include protein transduction domains and fusogenic peptides.

For example, the efficiency of particle delivery systems can also be improved by the attachment of functional ligands to the particle surface. Potential ligands include, but are not limited to, small molecules, cell-penetrating peptides (CPPs), targeting peptides, antibodies or aptamers (Yu, et al., PLoS One., 6:e24077 (2011), Cu, et al., J Control Release, 156:258-264 (2011), Nie, et al., J Control Release, 138:64-70 (2009), Cruz, et al., J Control Release, 144:118-126 (2010)). Attachment of these moieties serves a variety of different functions; such as inducing intracellular uptake, endosome disruption, and delivery of the plasmid payload to the nucleus. There have been numerous methods employed to tether ligands to the particle surface. One approach is direct covalent attachment to the functional groups on PLGA NPs (Bertram, Acta Biomater. 5:2860-2871 (2009)). Another approach utilizes amphiphilic conjugates like avidin palmitate to secure biotinylated ligands to the NP surface (Fahmy, et al., Biomaterials, 26:5727-5736 (2005), Cu, et al., Nanomedicine, 6:334-343 (2010)). This approach produces particles with enhanced uptake into cells, but reduced pDNA release and gene transfection, which is likely due to the surface modification occluding pDNA release. In a similar approach, lipid-conjugated polyethylene glycol (PEG) is used as a multivalent linker of penetratin, a CPP, or folate (Cheng, et al., Biomaterials, 32:6194-6203 (2011)).

These methods, as well as other methods discussed herein, and others methods known in the art, can be combined to tune particle function and efficacy. In some preferred embodiments, PEG is used as a linker for linking functional molecules to particles. For example, DSPE-PEG(2000)-maleimide is commercially available and can be used utilized for covalently attaching functional molecules such as CPP.

“Protein Transduction Domain” or PTD refers to a polypeptide, polynucleotide, or organic or inorganic compounds that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle. PTA can be short basic peptide sequences such as those present in many cellular and viral proteins. Exemplary protein transduction domains that are well-known in the art include, but are not limited to, the Antennapedia PTD and the TAT (transactivator of transcription) PTD, poly-arginine, poly-lysine or mixtures of arginine and lysine, HIV TAT, 11 arginine residues, VP22 peptide, and an ANTp peptide or positively charged polypeptides or polynucleotides having 8-15 residues, preferably 9-11 residues. Short, non-peptide polymers that are rich in amines or guanidinium groups are also capable of carrying molecules crossing biological membranes. Penetratin and other derivatives of peptides derived from antennapedia (Cheng, et al., Biomaterials, 32(26):6194-203 (2011) can also be used. Results show that penetratin in which additional Args are added, further enhances uptake and endosomal escape, and IKK NBD, which has an antennapedia domain for permeation as well as a domain that blocks activation of NFkB and has been used safely in the lung for other purposes (von Bismarck, et al., Pulmonary Pharmacology & Therapeutics, 25(3):228-35 (2012), Kamei, et al., Journal Of Pharmaceutical Sciences, 102(11):3998-4008 (2013)).

A “fusogenic peptide” is any peptide with membrane destabilizing abilities. In general, fusogenic peptides have the propensity to form an amphiphilic alpha-helical structure when in the presence of a hydrophobic surface such as a membrane. The presence of a fusogenic peptide induces formation of pores in the cell membrane by disruption of the ordered packing of the membrane phospholipids. Some fusogenic peptides act to promote lipid disorder and in this way enhance the chance of merging or fusing of proximally positioned membranes of two membrane enveloped particles of various nature (e.g. cells, enveloped viruses, liposomes). Other fusogenic peptides may simultaneously attach to two membranes, causing merging of the membranes and promoting their fusion into one. Examples of fusogenic peptides include a fusion peptide from a viral envelope protein ectodomain, a membrane-destabilizing peptide of a viral envelope protein membrane-proximal domain from the cytoplasmic tails.

Other fusogenic peptides often also contain an amphiphilic-region. Examples of amphiphilic-region containing peptides include: melittin, magainins, the cytoplasmic tail of HIV1 gp41, microbial and reptilian cytotoxic peptides such as bomolitin 1, pardaxin, mastoparan, crabrolin, cecropin, entamoeba, and staphylococcal .alpha.-toxin; viral fusion peptides from (1) regions at the N terminus of the transmembrane (TM) domains of viral envelope proteins, e.g. HIV-1, SIV, influenza, polio, rhinovirus, and coxsackie virus; (2) regions internal to the TM ectodomain, e.g. semliki forest virus, sindbis virus, rota virus, rubella virus and the fusion peptide from sperm protein PH-30: (3) regions membrane-proximal to the cytoplasmic side of viral envelope proteins e.g. in viruses of avian leukosis (ALV), Feline immunodeficiency (FIV), Rous Sarcoma (RSV), Moloney murine leukemia virus (MoMuLV), and spleen necrosis (SNV).

In particular embodiments, a functional molecule such as a CPP is covalently linked to DSPE-PEG-maleimide functionalized particles such as PBAE/PLGA blended particles using known methods such as those described in Fields, et al., J Control Release, 164(1):41-48 (2012). For example, DSPE-PEG-function molecule can be added to the 5.0% PVA solution during formation of the second emulsion. In some embodiments, the loading ratio is about 5 nmol/mg ligand-to-polymer ratio.

In some embodiments, the functional molecule is a CPP such as those above, or mTAT (HIV-1 (with histidine modification) (Yamano, et al., J Control Release, 152:278-285 (2011)); or bPrPp (Bovine prion) (Magzoub, et al., Biochem Biophys Res Commun., 348:379-385 (2006)); or MPG (Synthetic chimera: SV40 Lg T. Ant.+HIV gb41 coat) (Endoh, et al., Adv Drug Deliv Rev., 61:704-709 (2009)).

### IV. Methods of Manufacture

### A. Methods of Making Particles

The particle compositions described herein can be prepared by a variety of methods.

### 1. Polycations

In some embodiments, the nucleic acid is first complexed to a polycation. Complexation can be achieved by mixing the nucleic acids and polycations at an appropriate molar ratio. When a polyamine is used as the polycation species, it is useful to determine the molar ratio of the polyamine nitrogen to the polynucleotide phosphate (N/P ratio). In a preferred embodiment, nucleic acids and polyamines are mixed together to form a complex at an N/P ratio of between approximately 8:1 to 15:1. The volume of polyamine solution required to achieve particular molar ratios can be determined according to the following formula:

$${V}_{NH 2} = \frac{{C}_{nucacid , final} \times {M}_{w , nucacid} / {C}_{nucacid , final} \times {M}_{w , P} \times {Φ}_{N : P} \times Φ {V}_{final}}{{C}_{NH 2} / {M}_{w , NH 2}}$$

where M_{w, nucacid}=molecular weight of nucleic acid, M_{w,P}=molecular weight of phosphate groups of the nucleic acid, Φ_{N:P}=N:P ratio (molar ratio of nitrogens from polyamine to the ratio of phosphates from the nucleic acid), C_{NH2}, stock=concentration of polyamine stock solution, and M_{w,NH2}=molecular weight per nitrogen of polyamine.

Polycation complexation with nucleic acids can be achieved by mixing solutions containing polycations with solutions containing nucleic acids. The mixing can occur at any appropriate temperature. In one embodiment, the mixing occurs at room temperature. The mixing can occur with mild agitation, such as can be achieved through the use of a rotary shaker.

### 2. Exemplary Preferred Methods of Manufacture

In preferred embodiments, the particles are formed by a double-emulsion solvent evaporation technique, such as is disclosed in U.S. Published Application No. 2011/0008451 or U.S. Published Application No. 2011/0268810, each of which is a specifically incorporated by reference in its entirety, or Fahmy, et al., Biomaterials, 26:5727-5736, (2005), or McNeer, et al., Mol. Ther. 19, 172-180 (2011)). In this technique, the nucleic acids or nucleic acid/polycation complexes are reconstituted in an aqueous solution. Nucleic acid and polycation amounts are discussed in more detail below and can be chosen, for example, based on amounts and ratios disclosed in U.S. Published Application No. 2011/0008451 or U.S. Published Application No. 2011/0268810, or used by McNeer, et al., (McNeer, et al., Mol. Ther. 19, 172-180 (2011)), or by Woodrow et al. for small interfering RNA encapsulation (Woodrow, et al., Nat Mater, 8:526-533 (2009)). This aqueous solution is then added dropwise to a polymer solution of a desired polymer dissolved in an organic solvent to form the first emulsion.

This mixture is then added dropwise to solution containing a surfactant, such as polyvinyl alcohol (PVA) and sonicated to form the double emulsion. The final emulsion is then poured into a solution containing the surfactant in an aqueous solution and stirred for a period of time to allow the dichloromethane to evaporate and the particles to harden. The concentration of the surfactant used to form the emulsion, and the sonication time and amplitude can been optimized according to principles known in the art for formulating particles with a desired diameter. The particles can be collected by centrifugation. If it is desirable to store the particles for later use, they can be rapidly frozen, and lyophilized.

In preferred embodiments the particles are PLGA particles. In a particular exemplary protocol, nucleic acid (such as PNA, DNA, or PNA-DNA) with or without a polycation (such as spermidine) are dissolved in DNAse/RNAse free H₂O. Encapsulant in H₂O can be added dropwise to a polymer solution of 50:50 ester-terminated PLGA dissolved in dichloromethane (DCM), then sonicated to form the first emulsion. This emulsion can then be added dropwise to 5% polyvinyl alcohol, then sonicated to form the second emulsion. This mixture can be poured into 0.3% polyvinyl alcohol, and stirred at room temperature to form particles. Particles can then be collected and washed with, for example H₂O, collected by centrifugation, and then resuspended in H₂O, frozen at −80° C., and lyophilized. Particles can be stored at −20° C. following lyophilization.

Additional techniques for encapsulating the nucleic acid and polycation complex into polymeric particles are described below.

### 3. Solvent Evaporation

In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid particles. The resulting particles are washed with water and dried overnight in a lyophilizer. Particles with different sizes (0.5-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which can be performed in completely anhydrous organic solvents, are more useful.

### 4. Interfacial Polycondensation

Interfacial polycondensation is used to microencapsulate a core material in the following manner. One monomer and the core material are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

### 5. Solvent Evaporation Microencapsulation

In solvent evaporation microencapsulation, the polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid microcapsules containing core material.

The solvent evaporation process can be used to entrap a liquid core material in a polymer such as PLA, PLA/PGA copolymer, or PLA/PCL copolymer microcapsules. The polymer or copolymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the polymer or copolymer to phase separate and migrate towards the surface of the core material droplets. This phase-separated solution is then transferred into an agitated volume of nonsolvent, causing any remaining dissolved polymer or copolymer to precipitate and extracting any residual solvent from the formed membrane. The result is a microcapsule composed of polymer or copolymer shell with a core of liquid material.

Solvent evaporation microencapsulation can result in the stabilization of insoluble active agent particles in a polymeric solution for a period of time ranging from 0.5 hours to several months. Stabilizing an insoluble pigment and polymer within the dispersed phase (typically a volatile organic solvent) can be useful for most methods of microencapsulation that are dependent on a dispersed phase, including film casting, solvent evaporation, solvent removal, spray drying, phase inversion, and many others.

The stabilization of insoluble active agent particles within the polymeric solution could be critical during scale-up. By stabilizing suspended active agent particles within the dispersed phase, the particles can remain homogeneously dispersed throughout the polymeric solution as well as the resulting polymer matrix that forms during the process of microencapsulation.

Solvent evaporation microencapsulation (SEM) have several advantages. SEM allows for the determination of the best polymer-solvent-insoluble particle mixture that will aid in the formation of a homogeneous suspension that can be used to encapsulate the particles. SEM stabilizes the insoluble particles or pigments within the polymeric solution, which will help during scale-up because one will be able to let suspensions of insoluble particles or pigments sit for long periods of time, making the process less time-dependent and less labor intensive. SEM allows for the creation of particles that have a more optimized release of the encapsulated material.

### 6. Hot Melt Microencapsulation

In this method, the polymer is first melted and then mixed with the solid particles. The mixture is suspended in a non-miscible solvent (like silicon oil), and, with continuous stirring, heated to 5° C. above the melting point of the polymer. Once the emulsion is stabilized, it is cooled until the polymer particles solidify. The resulting particles are washed by decantation with petroleum ether to give a free-flowing powder. Particles with sizes between 0.5 to 1000 microns are obtained with this method. The external surfaces of spheres prepared with this technique are usually smooth and dense. This procedure is used to prepare particles made of polyesters and polyanhydrides. However, this method is limited to polymers with molecular weights between 1,000-50,000.

### 7. Solvent Removal Microencapsulation

In solvent removal microencapsulation, the polymer is typically dissolved in an oil miscible organic solvent and the material to be encapsulated is added to the polymer solution as a suspension or solution in organic solvent. Surface active agents can be added to improve the dispersion of the material to be encapsulated. An emulsion is formed by adding this suspension or solution to vigorously stirring oil, in which the oil is a nonsolvent for the polymer and the polymer/solvent solution is immiscible in the oil. The organic solvent is removed by diffusion into the oil phase while continuing to stir. Solvent removal results in precipitation of the polymer, forming solid microcapsules containing core material.

### 8. Phase Separation Microencapsulation

In phase separation microencapsulation, the material to be encapsulated is dispersed in a polymer solution with stirring. While continually stirring to uniformly suspend the material, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

### 9. Spontaneous Emulsification

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

### 10. Coacervation

Encapsulation procedures for various substances using coacervation techniques have been described in the prior art, for example, in GB-B-929 406; GB-B-929 401; U.S. Pat. Nos. 3,266,987; 4,794,000 and 4,460,563. Coacervation is a process involving separation of colloidal solutions into two or more immiscible liquid layers (Ref Dowben, R. General Physiology, Harper & Row, New York, 1969, pp. 142-143.). Through the process of coacervation compositions comprised of two or more phases and known as coacervates may be produced. The ingredients that comprise the two phase coacervate system are present in both phases; however, the colloid rich phase has a greater concentration of the components than the colloid poor phase.

### 11. Solvent Removal

This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. Particles that range between 1-300 microns can be obtained by this procedure. The external morphology of spheres produced with this technique is highly dependent on the type of polymer used.

### 12. Spray-Drying

In this method, the polymer is dissolved in organic solvent. A known amount of the active drug is suspended (insoluble drugs) or co-dissolved (soluble drugs) in the polymer solution. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting=15, pump setting=10 mL/minute, spray flow=600 Nl/hr, and nozzle diameter=0.5 mm. Particles ranging between 1-10 microns are obtained with a morphology which depends on the type of polymer used.

### 13. Nanoprecipitation

In nanoprecipitation, the polymer and nucleic acids are co-dissolved in a selected, water-miscible solvent, for example DMSO, acetone, ethanol, acetone, etc. In a preferred embodiment, nucleic acids and polymer are dissolved in DMSO. The solvent containing the polymer and nucleic acids is then drop-wise added to an excess volume of stirring aqueous phase containing a stabilizer (e.g., poloxamer, Pluronic®, and other stabilizers known in the art). Particles are formed and precipitated during solvent evaporation. To reduce the loss of polymer, the viscosity of the aqueous phase can be increased by using a higher concentration of the stabilizer or other thickening agents such as glycerol and others known in the art. Lastly, the entire dispersed system is centrifuged, and the nucleic acid-loaded polymer particles are collected and optionally filtered. Nanoprecipitation-based techniques are discussed in, for example, U.S. Pat. No. 5,118,528.

Advantages to nanoprecipitation include: the method can significantly increase the encapsulation efficiency of drugs that are polar yet water-insoluble, compared to single or double emulsion methods (Alshamsan, Saudi Pharmaceutical Journal, 22(3):219-222 (2014)). No emulsification or high shear force step (e.g., sonication or high-speed homogenization) is involved in nanoprecipitation, therefore preserving the conformation of nucleic acids. Nanoprecipitation relies on the differences in the interfacial tension between the solvent and the nonsolvent, rather than shear stress, to produce particles. Hydrophobicity of the drug will retain it in the instantly-precipitating particles; the un-precipitated polymer due to equilibrium is “lost” and not in the precipitated particle form.

### B. Molecules to be Encapsulated or Attached to the Surface of the Particles

There are two principle groups of molecules to be encapsulated or attached to the polymer, either directly or via a coupling molecule: targeting molecules, attachment molecules and therapeutic, nutritional, diagnostic or prophylactic agents. These can be coupled using standard techniques. The targeting molecule or therapeutic molecule to be delivered can be coupled directly to the polymer or to a material such as a fatty acid which is incorporated into the polymer.

Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in two ways. The first is during the preparation of the particles, for example during the emulsion preparation of particles by incorporation of stabilizers with functional chemical groups. Example 1 demonstrates this type of process whereby functional amphiphilic molecules are inserted into the particles during emulsion preparation.

A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This second class also includes a process whereby amphiphilic molecules such as fatty acids, lipids or functional stabilizers may be passively adsorbed and adhered to the particle surface, thereby introducing functional end groups for tethering to ligands.

In the preferred embodiment, the surface is modified to insert amphiphilic polymers or surfactants that match the polymer phase HLB or hydrophile-lipophile balance, as demonstrated in the following example. HLBs range from 1 to 15. Surfactants with a low HLB are more lipid loving and thus tend to make a water in oil emulsion while those with a high HLB are more hydrophilic and tend to make an oil in water emulsion. Fatty acids and lipids have a low HLB below 10. After conjugation with target group (such as hydrophilic avidin), HLB increases above 10. This conjugate is used in emulsion preparation. Any amphiphilic polymer with an HLB in the range 1-10, more preferably between 1 and 6, most preferably between 1 and up to 5, can be used. This includes all lipids, fatty acids and detergents.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of molecules to the polymer.

Coupling is preferably by covalent binding but it may also be indirect, for example, through a linker bound to the polymer or through an interaction between two molecules such as strepavidin and biotin. It may also be by electrostatic attraction by dip-coating.

The molecules to be delivered can also be encapsulated into the polymer using double emulsion solvent evaporation techniques, such as that described by Luo et al., Controlled DNA delivery system, Phar. Res., 16: 1300-1308 (1999).

### C. Exemplary Particle Formulations

The particle formulation can be selected based on the considerations including the targeted tissue or cells. For example, in embodiments directed to treatment of treating or correcting beta-thalassemia (e.g. when the target cells are, for example, hematopoietic stem cells), a preferred particle formulation is PLGA or PACE.

Other preferred particle formulations, particularly preferred for treating cystic fibrosis, are described in McNeer, et al., Nature Commun., 6:6952. doi: 10.1038/ncomms7952 (2015), and Fields, et al., Adv Healthc Mater., 4(3):361-6 (2015). doi: 10.1002/adhm.201400355 (2015) Epub 2014. Such particles are composed of a blend of Poly(beta-amino) esters (PBAEs) and poly(lactic-co-glycolic acid) (PLGA). Poly(beta-amino) esters (PBAEs) are degradable, cationic polymers synthesized by conjugate (Michael-like) addition of bifunctional amines to diacrylate esters (Lynn, Langer R, editor. J Am Chem Soc. 2000. pp. 10761-10768). PBAEs appear to have properties that make them efficient vectors for gene delivery. These cationic polymers are able to condense negatively charged pDNA, induce cellular uptake, and buffer the low pH environment of endosomes leading to DNA escape (Lynn, Langer R, editor. J Am Chem Soc. 2000. pp. 10761-10768, and Green, Acc Chem Res., 41(6):749-759 (2008)). PBAEs have the ability to form hybrid particles with other polymers, which allows for production of solid, stable and storable particles. For example, blending cationic PBAE with PLGA produced highly loaded pDNA particles. The addition of PBAE to PLGA resulted in an increase in gene transfection in vitro and induced antigen-specific tumor rejection in a murine model (Little, et al. Proc Natl Acad Sci USA., 101:9534-9539 (2004), Little, et al., J Control Release, 107:449-462 (2005)).

Therefore, in some embodiments, the particles utilized to deliver the compositions are composed of a blend of PBAE and a second polymer one of those discussed above. In some embodiments, the particles are composed of a blend of PBAE and PLGA.

PLGA and PBAE/PLGA blended particles loaded with gene editing technology can be formulated using a double-emulsion solvent evaporation technique such as that described in detail above, and in McNeer, et al., Nature Commun., 6:6952. doi: 10.1038/ncomms7952 (2015), and Fields, et al., Adv Healthc Mater., 4(3):361-6 (2015). doi: 10.1002/adhm.201400355 (2015) Epub 2014. Poly(beta amino ester) (PBAE) can synthesized by a Michael addition reaction of 1,4-butanediol diacrylate and 4,4′-trimethylenedipiperidine as described in Akinc, et al., Bioconjug Chem., 14:979-988 (2003). In some embodiments, PBAE blended particles such as PLGA/PBAE blended particles, contain between about 1 and 99, or between about 1 and 50, or between about 5 and 25, or between about 5 and 20, or between about 10 and 20, or about 15 percent PBAE (wt %). In particular embodiments, PBAE blended particles such as PLGA/PBAE blended particles, contain about 50, 45, 40, 35, 30, 25, 20, 15, 10, or 5% PBAE (wt %). Solvent from these particles in PVA as discussed above, and in some cases may continue overnight. PLGA/PBAE/MPG nanoparticles was shown to produce significantly greater nanoparticle association with airway epithelial cells than PLGA nanoparticles (Fields, et al., Advanced Healthcare Materials, 4:361-366 (2015)).

### X. Methods of Use

### A. Methods of Treatment

The methods most typically include in utero delivery of at least one active agent to an embryo or fetus in need thereof. In some embodiments, the methods of administration are used to deliver an active agent such as a therapeutic, nutritional, diagnostic, or prophylactic agents. The active agents can be small molecule active agents or biomacromolecules, such as proteins, polypeptides, or nucleic acids. In some embodiments, two or more active agent are delivered using an in utero delivery method. In particular embodiments, at least one of the active agents is a gene editing technology.

The active agent is typically administered in an effective amount to a subject in need thereof. The effective amount or therapeutically effective amount can be a dosage sufficient to treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder.

A composition including active agent can be administered to a specific location, organ, or tissue of the fetus or embryo. The composition can have a defined release profile. In some embodiments the composition is incapable of crossing the placenta. When two or more active agents are used, the agents or their particles can have the same targeting agent, or different targeting agents, wherein the different targeting agents are capable of specifically binding to the same or different targets on or in the fetus or embryo. When two or more compositions are used, the release profiles for the different active agents can be the same or different.

The compositions can be administered or otherwise contacted with target cells once, twice, or three time daily; one, two, three, four, five, six, seven times a week, one, two, three, four, five, six, seven or eight times a month. For example, in some embodiments, the composition is administered every two or three days, or on average about 2 to about 4 times about week.

In some embodiments, the methods include contacting a cell with an effective amount of gene editing composition, alone or in combination with a potentiating agent, to modify the cell's genome. In some embodiments, the active agent is internalized by the cell. In some embodiments, the active agent need not be internalized. In some embodiments, the active agent serves as a paracrine factor. As discussed in more detail below, the contacting can occur ex vivo or in vivo. Thus, the gene editing composition is administered in vivo, or ex vivo edited cells can be administered to a subject in need thereof. In some embodiments, the method includes contacting a population of target cells with an effective amount of gene editing composition, preferably in combination with a potentiating agent, to modify the genomes of a sufficient number of cells to achieve a therapeutic result. In some embodiments, cells are not administered.

In some embodiments, when the gene editing technology is triplex forming molecules, the molecules can be administered in an effective amount to induce formation of a triple helix at the target site. An effective amount of gene editing technology such as triplex-forming molecules may also be an amount effective to increase the rate of recombination of a donor fragment relative to administration of the donor fragment in the absence of the gene editing technology. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids. The precise dosage will vary according to a variety of factors such as subject-dependent variables (e.g., age, immune system health, clinical symptoms etc.). Exemplary symptoms, pharmacologic, and physiologic effects are discussed in more detail below.

In some embodiments, a potentiating agent is administered to the subject prior to administration of the gene editing technology to the subject. The potentiating agent can be administered to the subject, for example, 1, 2, 3, 4, 5, 6, 8, 10, 12, 18, or 24 hours, or 1, 2, 3, 4, 5, 6, or 7 days, or any combination thereof prior to administration of the gene editing technology to the subject.

In some embodiments, a gene editing technology is administered to the subject prior to administration of a potentiating agent to the subject. The gene editing technology can be administered to the subject, for example, 1, 2, 3, 4, 5, 6, 8, 10, 12, 18, or 24 hours, or 1, 2, 3, 4, 5, 6, or 7 days, or any combination thereof prior to administration of the potentiating agent to the subject.

In some embodiments, the compositions are administered in an amount effective to induce gene modification in at least one target allele to occur at frequency of at least 0.1, 0.2. 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, or 25% of target cells. In some embodiments, particularly ex vivo applications, gene modification occurs in at least one target allele at a frequency of about 0.1-25%, or 0.5-25%, or 1-25% 2-25%, or 3-25%, or 4-25% or 5-25% or 6-25%, or 7-25%, or 8-25%, or 9-25%, or 10-25%, 11-25%, or 12-25%, or 13%-25% or 14%-25% or 15-25%, or 2-20%, or 3-20%, or 4-20% or 5-20% or 6-20%, or 7-20%, or 8-20%, or 9-20%, or 10-20%, 11-20%, or 12-20%, or 13%-20% or 14%-20% or 15-20%, 2-15%, or 3-15%, or 4-15% or 5-15% or 6-15%, or 7-15%, or 8-15%, or 9-15%, or 10-15%, 11-15%, or 12-15%, or 13%-15% or 14%-15%.

In some embodiments, particularly in vivo applications, gene modification occurs in at least one target allele at a frequency of about 0.1% to about 10%, or about 0.2% to about 10%, or about 0.3% to about 10%, or about 0.4% to about 10%, or about 0.5% to about 10%, or about 0.6% to about 10%, or about 0.7% to about 10%, or about 0.8% to about 10%, or about 0.9% to about 10%, or about 1.0% to about 10%, or about 1.1% to about 10%, or about 1.1% to about 10%, 1.2% to about 10%, or about 1.3% to about 10%, or about 1.4% to about 10%, or about 1.5% to about 10%, or about 1.6% to about 10%, or about 1.7% to about 10%, or about 1.8% to about 10%, or about 1.9% to about 10%, or about 2.0% to about 10%, or about 2.5% to about 10%, or about 3.0% to about 10%, or about 3.5% to about 10%, or about 4.0% to about 10%, or about 4.5% to about 10%, or about 5.0% to about 10%.

In some embodiments, gene modification occurs with low off-target effects. In some embodiments, off-target modification is undetectable using routine analysis such as those described in the Examples below. In some embodiments, off-target incidents occur at a frequency of 0-1%, or 0-0.1%, or 0-0.01%, or 0-0.001%, or 0-0.0001%, or 0-0000.1%, or 0-0.000001%. In some embodiments, off-target modification occurs at a frequency that is about 10², 10³, 10⁴, or 10⁵-fold lower than at the target site.

### Exemplary Dosages

In general, by way of example only, dosage forms useful in the methods can include doses in the range of about 10² to about 10⁵⁰, or about 10⁵ to about 10⁴⁰, or about 10¹⁰ to about 10³⁰, or about 10¹² to about 10²⁰ copies of the gene editing technology per dose. In particular embodiments, about 10¹³, 10¹⁴, 10¹⁵, 10¹⁶, or 10¹⁷ copies of gene editing technology are administered to a subject in need thereof.

In other embodiments, dosages are expressed in moles. For example, in some embodiments, the dose of gene editing technology is about 0.1 nmol to about 100 nmol, or about 0.25 nmol to about 50 nmol, or about 0.5 nmol to about 25 nmol, or about 0.75 nmol to about 7.5 nmol.

In other embodiments, dosages are expressed in molecules per target cells. For example, in some embodiments, the dose of gene editing technology is about 10² to about 10⁵⁰, or about 10⁵ to about 10¹⁵, or about 10⁷ to about 10¹², or about 10⁸ to about 10¹¹ copies of the gene editing technology per target cell.

In other embodiments, dosages are expressed in mg/kg, particularly when the expressed as an in vivo dosage of an active agent such as a growth factors or a gene editing composition packaged in a particle with or without functional molecules. Dosages for active agents can be, for example, between 0.1 mg/kg and about 1,000 mg/kg, or 0.5 mg/kg and about 1,000 mg/kg, or 1 mg/kg and about 1,000 mg/kg, or about 10 mg/kg and about 500 mg/kg, or about 20 mg/kg and about 500 mg/kg per dose, or 20 mg/kg and about 100 mg/kg per dose, or 25 mg/kg and about 75 mg/kg per dose, or about 25, 30, 35, 40, 45, 50, 55, 60, 65, 70, or 75 mg/kg per dose.

In some of the Examples below, intra-amniotic injections were performed with 20 μl of 9 mg/ml PNA/DNA nanoparticles, or intravenously with 15 μl of either 9 mg/ml or 12 mg/ml PNA/DNA NPs, correlating to doses of 300 mg kg⁻¹ or 400 mg kg⁻¹, respectively.

In some Examples below, for in uterine delivery by injection of alginate particles, each fetus received 30 μL of 20 mg/mL alginate microparticles suspended in PBS. For the arm treated with bFGF alone, bFGF was diluted to match the expected concentration of bFGF in the Alginate-HSA-bFGF, which was approximately 0.4 μg/μL or 12 μg per injection.

In other embodiments, dosages are expressed in mg/ml, particularly when expressed as an ex vivo dosage of active agent such a growth factor or gene editing composition packaged in a particle with or without functional molecules. Dosages can be, for example 0.01 mg/ml to about 100 mg/ml, or about 0.5 mg/ml to about 50 mg/ml, or about 1 mg/ml to about 10 mg/ml per dose to a cell population of 10⁶ cells.

As discussed above, gene editing technology can be administered without, but is preferably administered with at least one donor oligonucleotide. Such donors can be administered at similar dosages as the gene editing technology. Compositions should include an amount of donor fragment effective to recombine at the target site in the presence of a gene editing technology such as triplex forming molecules.

With respect to potentiating agents, preferably an amount effective to increase gene modification when used in combination with a gene modifying technology, compared to using the gene modifying technology in the absence of the potentiating agent.

Exemplary dosages for SCF include, between about 0.01 mg/kg and about 250 mg/kg, or about 0.1 mg/kg and about 100 mg/kg, or about 0.5 mg/kg and about 50 mg/kg, or about 0.75 mg/kg to about 10 mg/kg.

Dosages for CHK1 inhibitors are known in the art, and many of these are in clinical trial. Accordingly, the dosage can be selected by the practitioner based on known, preferred humans dosages. In preferred embodiments, the dosage is below the lowest-observed-adverse-effect level (LOAEL), and is preferably a no observed adverse effect level (NOAEL) dosage.

Dosage units including an effective amount of the compositions are also provided. The dosage can be lower than the effective dosage of the same composition when administered to treat the fetus after birth, as a child, or as an adult. The dosage can be effective to treat or prevent a disease or disorder in the fetus.

### 1. In Vivo Gene Therapy

The compositions can be administered directly to a subject for in vivo gene therapy.

a. Pharmaceutical Formulations

The compositions are preferably employed for therapeutic uses in combination with a suitable pharmaceutical carrier. Such compositions include an effective amount of the composition, and a pharmaceutically acceptable carrier or excipient.

It is understood by one of ordinary skill in the art that nucleotides administered in vivo are taken up and distributed to cells and tissues (Huang, et al., FEBS Lett., 558(1-3):69-73 (2004)). For example, Nyce, et al. have shown that antisense oligodeoxynucleotides (ODNs) when inhaled bind to endogenous surfactant (a lipid produced by lung cells) and are taken up by lung cells without a need for additional carrier lipids (Nyce, et al., Nature, 385:721-725 (1997)). Small nucleic acids are readily taken up into T24 bladder carcinoma tissue culture cells (Ma, et al., Antisense Nucleic Acid Drug Dev., 8:415-426 (1998)).

The compositions including active agents such as triplex-forming molecules, such as TFOs and PNAs, and donor fragments may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles, nanoparticles, or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example, in Sambrook et al., Molecular Cloning: A Laboratory Manual, Cold Spring Harbor Laboratory, New York (1989); and Ausubel, et al., Current Protocols in Molecular Biology, John Wiley & Sons, New York (1994). Such nucleic acid delivery systems include the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. The nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases. In addition, sterile, fixed oils are conventionally employed as a solvent or suspending medium. For this purpose any bland fixed oil including synthetic mono- or di-glycerides may be employed. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

In some embodiments, the compositions include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the triplex-forming molecules and/or donor oligonucleotides are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett., 14(19):4975-4977 (2004)) and in vivo (Soutschek, et al., Nature, 432(7014):173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol., 59(11):1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

b. Methods of Administration
i. In Utero Administration

Although genetic diseases can be diagnosed early during pregnancy, many continue to cause considerable pediatric morbidity and mortality. Early intervention through intrauterine gene editing, however, can correct the genetic defect, allowing for normal organ development, functional disease improvement, or cure. The Examples herein demonstrate safe intravenous and intra-amniotic delivery of polymeric nanoparticles and microparticles to fetal mouse and rat tissues at selected gestational ages with no effect on survival or postnatal growth. In utero introduction of nanoparticles containing peptide nucleic acids (PNAs) and donor DNAs corrected a disease-causing mutation in the β-globin gene in a mouse model of human β-thalassemia, yielding sustained postnatal elevation of blood hemoglobin levels into the normal range, reduced reticulocyte counts, and reversal of splenomegaly, with no detected off-target mutations in partially homologous loci. The in vivo data below shows that 500 days after birth, in utero treated mice had 100% survival, in contrast to just 69% survival in the untreated group. The data also shows that gene modification is some cells of the fetus can persist for at least 9 months.

Thus, safe and versatile methods of fetal therapy including genetic therapy for human monogenic disorders are provided. The methods typically include in utero administration to an embryo or fetus of an effective amount of gene editing composition. Routes of administration include traditional routes such as to intramuscular, intraperitoneal, spinal canal, lumina, lateral cerebral ventricles, puncture of the fetal heart, placental cord insertion, the intrahepatic umbilical vein, intraplacental, yolk sac vessels, intra-organ (e.g., other organs and tissues, including brain, muscle, heart, etc.) and other disclosed herein and in Waddington, et al., “In Utero gene therapy: current challenges and perspectives,” Molecular Therapy, Volume 11, Issue 5, May 2005, Pages 661-676.

In some embodiments the route of administration is via an intravenous or intra-amniotic injection or infusion. The compositions can be administered during in utero surgery. The experiments below show that administration of nanoparticulate compositions to fetal mice results in particle retention within the fetuses with no detectable particle accumulation in the maternal mouse. Thus, the methods can used to deliver effective amounts of compositions to the embryo or fetus, or cells thereof, without delivering an effective amount of the composition of the mother of the embryo or fetus, or her cells. For example, in some gene editing embodiments, the target embryo or fetus is contacted with an effective amount of gene editing composition to alter the genomes of a sufficient number of its cells to reduce or prevent one or more symptoms of a target genetic disease. At the same time, the amount, route of delivery, or combination thereof may not be effective to alter genome of a sufficient number of her cells to change her phenotype.

In some methods the compositions can be administered by injection or infusion intravascularly into the vitelline vein, or umbilical vein, or an artery such as the vitelline artery of an embryo or fetus. In the experiments described below, intra-vitelline vein delivery of fluorescent PLGA nanoparticles (NPs) resulted in widespread fetal particle distribution at both E15.5 and E16.5 with the most abundant NP accumulation in the fetal liver. It is believed that the substantial accumulation of NPs in the fetal liver observed occurs because extraembryonic vitelline veins anastomose to form the portal circulation.

Additionally (to injection into the vitelline vein) or alternatively, the same or different compositions can be administered by injection or infusion into the amniotic cavity. During physiologic mammalian fetal development, the fetus breaths amniotic fluid into and out of the developing lungs, providing the necessary forces to direct lung development and growth. Developing fetuses additionally swallow amniotic fluid, which aids the formation of the gastrointestinal tract. The experiments below show that introduction of a nanoparticulate composition into the amniotic fluid at gestational ages after the onset of fetal breathing and swallowing resulted in delivery to the lung and gut, respectively, with increased intensity of accumulation at the later gestational ages, while administration before the onset of fetal breathing and swallowing did not lead to any detectable particle accumulation within the fetus.

The methods can be carried out at any time it is technically feasible to do so and the method are efficacious.

Fetal surgeons and maternal fetal medicine physicians can safely access the amniotic cavity for amniocentesis and cannulate umbilical vessels for fetal blood transfusions under ultrasound guidance as early as 13 weeks of gestation in humans (Bang, et al., Br Med J(Clin Res Ed), 284, 373-374 (1982), Kempe et al., Ultrasound Obstet Gynecol, 29, 226-228 (2007)). Under current medical guidelines, amniocentesis can be performed safely at 15 to 20 weeks of gestation. These procedures have been used in clinical practice since the 1980's and carry a very low risk of fetal loss (~1%) (Bang, et al., Br Med J(Clin Res Ed), 284, 373-374 (1982), Van Kamp, et al., Am J Obstet Gynecol, 192, 171-177 (2005)). These procedures can be utilized for in utero delivery of the compositions in human and other animals. An injection involves a tiny puncture in the amniotic membrane; and the therapy could be delivered earlier and with relatively low risk of premature labor and fetal loss.

In a human, the process of injection can be performed in a manner similar to amniocentesis, during which an ultrasound-guided needle is inserted into the amniotic sac to withdraw a small amount of amniotic fluid for genetic testing. A glass pipette is an exemplary needle-like tool amenable for shape and size modification for piercing through the amniotic membrane via a tiny puncture, and dispensing formulation into the utero. In some of the Examples below, a pulled glass pipette with approximately 60 micron tip, 30 microliters of suspended microparticles are injected into the amniotic space surrounding the rat fetuses. Dimensions of the tool to deliver formulation into the amniotic space are variable depending on the subject. The uterus is then returned to its normal position within the abdomen, and the pregnant dam's abdominal wall is closed.

The composition can be administered to a fetus, embryo, or to the mother or other subject when the fetus or embryo is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 weeks of age.

In some embodiments, the methods are carried out at a gestational time point during which agents can be safely delivered via the umbilical vessels. In some methods in utero administration is carried out on or after the gestational equivalent of E15, E15.5, or E16 of a mouse (e.g., a human or mammal's gestational age equivalent to murine gestational age E15, E15.5, or E16). Typically intraamniotic injection is carried out on or after the gestational equivalent of E16 or E16.5, or on or after fetal breathing and/or swallowing has begun.

In other embodiments, intraamniotic injection is carried out on or after the gestational equivalent of E14, E15, E16, E17, E18, E19, E20, or E21 of a rat (e.g., a human or other mammal's gestational age equivalent to rat gestational age E14, E15, E16, E17, E18, E19, E20, or E21).

The compositions can be administered before damage has occurred or when only limited damage has occurred. In some embodiments, the compositions are administered before nerve damage occurs, (e.g., the limbs of the fetus or embryo are still moving).

ii. Additional Routes of Administration

Additional or alternative methods of administration can also be employed. In particular, the routes of administration already in use for nucleic acid therapeutics, along with formulations in current use, provide additional and alternative preferred routes of administration and formulation for the triplex-forming molecules described above. Preferably the compositions are injected into the organism undergoing genetic manipulation, such as an animal requiring gene therapy. The compositions can be administered by a number of routes including, but not limited to, oral, intravenous, intraperitoneal, intramuscular, transdermal, subcutaneous, topical, sublingual, rectal, intranasal, pulmonary, and other suitable means. The compositions can also be administered via liposomes. Such administration routes and appropriate formulations are generally known to those of skill in the art.

Administration of the formulations may be accomplished by any acceptable method which allows the gene editing compositions to reach their targets.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The compositions may be delivered in a manner which enables tissue-specific uptake of the agent and/or nucleotide delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the composition, over a certain time period, for example, hours, days, weeks, months or years. This may be accomplished, for example, by repeated administrations of a formulation or by a sustained or controlled release delivery system in which the compositions are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the oligonucleotides are contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the triplex-forming molecules and donor oligonucleotides. In addition, a pump-based hardware delivery system may be used to deliver one or more embodiments.

Examples of systems in which release occurs in bursts include systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

### 2. Ex Vivo Gene Therapy

In some embodiments, ex vivo gene therapy of cells is used for the treatment of a subject. For ex vivo gene therapy, cells are isolated, for example from a subject, and contacted ex vivo with the compositions to produce cells containing mutations in or adjacent to genes. In a preferred embodiment, the cells are isolated from the subject to be treated or from a syngenic host. Target cells are removed from a subject prior to contacting with a gene editing composition and preferably a potentiating factor. The cells can be hematopoietic progenitor or stem cells. In a preferred embodiment, the target cells are CD34⁺ hematopoietic stem cells. Hematopoietic stem cells (HSCs), such as CD34+ cells are multipotent stem cells that give rise to all the blood cell types including erythrocytes. Therefore, CD34+ cells can be isolated from a patient with, for example, thalassemia, sickle cell disease, or a lysosomal storage disease, the mutant gene altered or repaired ex-vivo using the compositions and methods, and the cells reintroduced back into the patient as a treatment or a cure.

Stem cells can be isolated and enriched by one of skill in the art. Methods for such isolation and enrichment of CD34⁺ and other cells are known in the art and disclosed for example in U.S. Pat. Nos. 4,965,204; 4,714,680; 5,061,620; 5,643,741; 5,677,136; 5,716,827; 5,750,397 and 5,759,793. As used herein in the context of compositions enriched in hematopoietic progenitor and stem cells, “enriched” indicates a proportion of a desirable element (e.g. hematopoietic progenitor and stem cells) which is higher than that found in the natural source of the cells. A composition of cells may be enriched over a natural source of the cells by at least one order of magnitude, preferably two or three orders, and more preferably 10, 100, 200 or 1000 orders of magnitude.

In humans, CD34⁺ cells can be recovered from cord blood, bone marrow or from blood after cytokine mobilization effected by injecting the donor with hematopoietic growth factors such as granulocyte colony stimulating factor (G-CSF), granulocyte-monocyte colony stimulating factor (GM-CSF), stem cell factor (SCF) subcutaneously or intravenously in amounts sufficient to cause movement of hematopoietic stem cells from the bone marrow space into the peripheral circulation. Initially, bone marrow cells may be obtained from any suitable source of bone marrow, e.g. tibiae, femora, spine, and other bone cavities. For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include Hepes, phosphate buffers, lactate buffers, etc.

Cells can be selected by positive and negative selection techniques. Cells can be selected using commercially available antibodies which bind to hematopoietic progenitor or stem cell surface antigens, e.g. CD34, using methods known to those of skill in the art. For example, the antibodies may be conjugated to magnetic beads and immunogenic procedures utilized to recover the desired cell type. Other techniques involve the use of fluorescence activated cell sorting (FACS). The CD34 antigen, which is found on progenitor cells within the hematopoietic system of non-leukemic individuals, is expressed on a population of cells recognized by the monoclonal antibody My-10 (i.e., express the CD34 antigen) and can be used to isolate stem cell for bone marrow transplantation. My-10 deposited with the American Type Culture Collection (Rockville, Md.) as HB-8483 is commercially available as anti-HPCA 1. Additionally, negative selection of differentiated and “dedicated” cells from human bone marrow can be utilized, to select against substantially any desired cell marker. For example, progenitor or stem cells, most preferably CD34+ cells, can be characterized as being any of CD3⁻, CD7⁻, CD8⁻, CD10⁻, CD14⁻, CD15⁻, CD19⁻, CD20⁻, CD33⁻, Class II HLA⁺ and Thy-1⁺.

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium including cell surface factors supporting the proliferation of stem cells. Stromal cells may be freed of hematopoietic cells employing appropriate monoclonal antibodies for removal of the undesired cells.

The isolated cells are contacted ex vivo with a combination of triplex-forming molecules and donor oligonucleotides in amounts effective to cause the desired mutations in or adjacent to genes in need of repair or alteration, for example the human beta-globin or α-L-iduronidase gene. These cells are referred to herein as modified cells. Methods for transfection of cells with oligonucleotides and peptide nucleic acids are well known in the art (Koppelhus, et al., Adv. Drug Deliv. Rev., 55(2): 267-280 (2003)). It may be desirable to synchronize the cells in S-phase to further increase the frequency of gene correction. Methods for synchronizing cultured cells, for example, by double thymidine block, are known in the art (Zielke, et al., Methods Cell Biol., 8:107-121 (1974)).

The modified cells can be maintained or expanded in culture prior to administration to a subject. Culture conditions are generally known in the art depending on the cell type. Conditions for the maintenance of CD34⁺ in particular have been well studied, and several suitable methods are available. A common approach to ex vivo multi-potential hematopoietic cell expansion is to culture purified progenitor or stem cells in the presence of early-acting cytokines such as interleukin-3. It has also been shown that inclusion, in a nutritive medium for maintaining hematopoietic progenitor cells ex vivo, of a combination of thrombopoietin (TPO), stem cell factor (SCF), and flt3 ligand (Flt-3L; i.e., the ligand of the flt3 gene product) was useful for expanding primitive (i.e., relatively non-differentiated) human hematopoietic progenitor cells in vitro, and that those cells were capable of engraftment in SCID-hu mice (Luens et al., 1998, Blood 91:1206-1215). In other known methods, cells can be maintained ex vivo in a nutritive medium (e.g., for minutes, hours, or 3, 6, 9, 13, or more days) including murine prolactin-like protein E (mPLP-E) or murine prolactin-like protein F (mPIP-F; collectively mPLP-E/IF) (U.S. Pat. No. 6,261,841). It will be appreciated that other suitable cell culture and expansion method can be used in accordance with the invention as well. Cells can also be grown in serum-free medium, as described in U.S. Pat. No. 5,945,337.

In another embodiment, the modified hematopoietic stem cells are differentiated ex vivo into CD4⁺ cells culture using specific combinations of interleukins and growth factors prior to administration to a subject using methods well known in the art. The cells may be expanded ex vivo in large numbers, preferably at least a 5-fold, more preferably at least a 10-fold and even more preferably at least a 20-fold expansion of cells compared to the original population of isolated hematopoietic stem cells.

In another embodiment cells for ex vivo gene therapy, the cells to be used can be dedifferentiated somatic cells. Somatic cells can be reprogrammed to become pluripotent stem-like cells that can be induced to become hematopoietic progenitor cells. The hematopoietic progenitor cells can then be treated with triplex-forming molecules and donor oligonucleotides as described above with respect to CD34⁺ cells to produce recombinant cells having one or more modified genes. Representative somatic cells that can be reprogrammed include, but are not limited to fibroblasts, adipocytes, and muscles cells. Hematopoietic progenitor cells from induced stem-like cells have been successfully developed in the mouse (Hanna, J. et al. Science, 318:1920-1923 (2007)).

To produce hematopoietic progenitor cells from induced stem-like cells, somatic cells are harvested from a host. In a preferred embodiment, the somatic cells are autologous fibroblasts. The cells are cultured and transduced with vectors encoding Oct4, Sox2, Klf4, and c-Myc transcription factors. The transduced cells are cultured and screened for embryonic stem cell (ES) morphology and ES cell markers including, but not limited to AP, SSEA1, and Nanog. The transduced ES cells are cultured and induced to produce induced stem-like cells. Cells are then screened for CD41 and c-kit markers (early hematopoietic progenitor markers) as well as markers for myeloid and erythroid differentiation.

The modified hematopoietic stem cells or modified induced hematopoietic progenitor cells are then introduced into a subject. Delivery of the cells may be effected using various methods and includes most preferably intravenous administration by infusion as well as direct depot injection into periosteal, bone marrow and/or subcutaneous sites.

The subject receiving the modified cells may be treated for bone marrow conditioning to enhance engraftment of the cells. The recipient may be treated to enhance engraftment, using a radiation or chemotherapeutic treatment prior to the administration of the cells. Upon administration, the cells will generally require a period of time to engraft. Achieving significant engraftment of hematopoietic stem or progenitor cells typically takes weeks to months.

A high percentage of engraftment of modified hematopoietic stem cells is not envisioned to be necessary to achieve significant prophylactic or therapeutic effect. It is expected that the engrafted cells will expand over time following engraftment to increase the percentage of modified cells. In some embodiments, the modified cells have a corrected α-L-iduronidase gene. Therefore, in a subject with Hurler syndrome, the modified cells are expected to improve or cure the condition. It is expected that engraftment of only a small number or small percentage of modified hematopoietic stem cells will be required to provide a prophylactic or therapeutic effect.

In preferred embodiments, the cells to be administered to a subject will be autologous, e.g. derived from the subject, or syngenic.

### B. Diseases to be Treated

Therapy including, but not limited to, genetic correction during pregnancy could provide treatment or cure of numerous diseases and allow normal fetal development. The method can treat or prevent a disease or disorder in the fetus or embryo. The disease or disorder can be a structural defect or genetic defect. The disease or disorder can be a fetal disease or disorder.

Suitable subject include, but are not limited to mammals such as a human or other primate, a rodent such as a mouse or rat, or an agricultural or domesticated animal such as a dog, cat, cow, horse, pig, or sheep.

In some embodiments, the composition does not enter the mother's bodily fluids or tissues.

The mother can genetically related to the fetus or genetically unrelated to the fetus or embryo. Thus, the mother can be a surrogate. The fetus or embryo and the mother have the same disease or disorder or be at risk for developing the same disease or disorder. The fetus or embryo can have a disease or disorder or be at risk for developing a disease or disorder that the mother neither has nor has any risk of developing.

The formulations can be delivered in a minimally invasive fashion through an intra-amniotic injection or intravenous injection. The methods can be performed throughout gestation. Intra-amniotic injection has an adverse event profile similar to amniocentesis and IA pharmacotherapy.

Although some rare congenital conditions are treated with intra-amniotic injection of free hormones (levothyroxine) or steroids (Hashimoto H, et al., Fetal Diagn Ther., 2006; 21(4):360-5; Ribault V, et al., The Journal of clinical endocrinology and metabolism, 2009; 94(10):3731-9.; Hui L, et al., Prenat Diagn. 2011; 31(7):735-43.; Hanono A, et al., J Matern Fetal Neonatal Med. 2009; 22(1):76-80.), the formulation and intraamniotic administration process allows therapeutic molecules (drugs, hormones, growth factors, etc.) to be encapsulated in biocompatible nano- or microparticles that would not cross the placenta. (Fields R J, et al., Advanced healthcare materials. 2014; Patel T, et al, Advanced drug delivery reviews. 2012; 64(7):701-5.; Fahmy™, et al., Nanomedicine. 2008; 3(3):343-55.)

The biocompatible micro- and nanoparticle formulation for the controlled delivery of therapeutic, prophylactic, and/or diagnostic agents (e.g., drugs, proteins, or DNA editing molecules) to specific targets on the developing fetus can also be used to treat a wide variety of diseases and disorders. For instance, one could use our procedure to treat aliments of the respiratory or gastrointestinal tracts in utero.

The Examples below show that delivery FGF growth factor in utero can correct a pathology associated with spina bifida. In some embodiments, repair of the MMC defect before secondary intrauterine nerve damage occurs is more beneficial than open fetal repair at 23 weeks gestational age. By inducing a skin or soft tissue water-tight covering of the defect early in development, MMC is converted into spina bifida occulta or at least secondary nerve injury is prevented until definitive postnatal repair can be performed. As spina bifida occulta is usually asymptomatic, this conversion could effectively be a cure for the devastating sequelae of spina bifida.

Other target diseases and disorders include, but are not limited to, muscular dystrophy, Type 2 diabetes, diseases of the liver such as Wilson's disease and hemochromatosis, or diseases of the central nervous system including Fredrich's ataxia, Huntington's disease, spinal muscular atrophy, tuberous sclerosis.

Thus, for example, in some embodiments, the fetus or embryo is suffering spina bifida, a respiratory defect, or a gastrointestinal defect, and the methods include administering an effective amount of a therapeutic composition to treat the spina bifida, a respiratory defect, or a gastrointestinal defect.

Candidates for in utero gene therapy include diseases corrected by replacement of an inactive or absent protein. Monogenic diseases that pose the risk of serious fetal, neonatal, and pediatric morbidity or mortality are particularly attractive targets for in utero gene editing. Exemplary disease targets include, but are not limited to, cystic fibrosis, Tay-Sachs disease, hematopoietic stem cell disorders (e.g., sickle cell, thalassemia), and others disclosed herein.

Attractive targets for in utero gene therapy also include those discussed in Schneider & Coutelle, Nature Medicine, 5, 256-257 (1999), a the Table from which is reproduced below.

Exemplary candidate diseases for prenatal gene therapy

| | Therapeutic gene | |
|---|---|---|
| Disease | product | Target cells |
| Cystic fibrosis | Cystic fibrosis | Bronchial and |
| | transmembrane | intestinal |
| | conductance regulator | epithelial cells |
| Ornithine transcarbanylas | Ornithine | Hepatocytes |
| deficiency | transcarbaronylase | |
| Glycogen storage | α1,4-glucosidase | muscle cells, |
| disorders: | | hepatocytes, |
| Pompe disease | | neurons |
| Sphingolipid storage | Glucocerebrosidase | Hematopietic |
| disorders: | β-N- | stem cells, |
| Gaucher disease | acetylhexosaminidase | muscle cells, |
| Tay-Sachs disease | cerebroxide sulfatase | fibroblasts, |
| Metachromatic | | neurons |
| leucodystrophy | α-L-idoronidase | hematopoietic |
| Mucopolysaccharide | | |
| storage disorders: | | |
| Horler disease | iduronate-2 sulfatase | stem cells, |
| Hunter disease | β-glucoronidase | fibroblasts, |
| Sky disease | | neurons |
| Muscular dystrophy type | Dystrophin | Muscle cells |
| Duchenne | α-globin chains of | fetal blood cells |
| α2-Thalassemia | | |
| Crigler-Najar disease type | hemoglobin | hepatocytes |
| 1 | billrubine | |
| | glucuronyltransferase | |
| Tyrosinemia type 1 | fumaryfacetoacetate | hepatocytes |
| Junctional epidermolysis | lyase | keratinocytes |
| | laminin-5 chains | |
| bullosa type Herlitz | Survival motor neuron | neurons |
| Spinal Muscular atrophy | | |
| type Werdnig-Hoffmann | protein or neurotrophin-3 | |

Gene therapy is apparent when studied in the context of human genetic diseases, for example, cystic fibrosis, hemophilia, globinopathies such as sickle cell anemia and beta-thalassemia, xeroderma pigmentosum, and lysosomal storage diseases, though the strategies are also useful for treating non-genetic disease such as HIV, in the context of ex vivo-based cell modification and also for in vivo cell modification. The compositions are especially useful to treat genetic deficiencies, disorders and diseases caused by mutations in single genes, for example, to correct genetic deficiencies, disorders and diseases caused by point mutations. If the target gene contains a mutation that is the cause of a genetic disorder, then the compositions can be used for mutagenic repair that may restore the DNA sequence of the target gene to normal. The target sequence can be within the coding DNA sequence of the gene or within an intron. The target sequence can also be within DNA sequences that regulate expression of the target gene, including promoter or enhancer sequences.

If the target gene is an oncogene causing unregulated proliferation, such as in a cancer cell, then the oligonucleotide is useful for causing a mutation that inactivates the gene and terminates or reduces the uncontrolled proliferation of the cell. The oligonucleotide is also a useful anti-cancer agent for activating a repressor gene that has lost its ability to repress proliferation. The target gene can also be a gene that encodes an immune regulatory factor, such as PD-1, in order to enhance the host's immune response to a cancer.

Programmed cell death protein 1, also known as PD-1 and CD279 (cluster of differentiation 279), is a protein encoded by the PDCD1 gene. PD-1 has two ligands: PD-L1 and PD-L2. PD-1 is expressed on a subset of thymocytes and up-regulated on T, B, and myeloid cells after activation (Agata, et al., Int. Immunol., 8:765-772 (1996)). PD-1 acts to antagonize signal transduction downstream of the TCR after it binds a peptide antigen presented by the major histocompatibility complex (MHC). It can function as an immune checkpoint, by preventing the activation of T-cells, which in turn reduces autoimmunity and promotes self-tolerance, but can also reduce the body's ability to combat cancer. The inhibitory effect of PD-1 to act through twofold mechanism of promoting apoptosis (programmed cell death) in antigen specific T-cells in lymph nodes while simultaneously reducing apoptosis in regulatory T cells (suppressor T cells). Compositions that block PD-1, the PD-1 inhibitors, activate the immune system to attack tumors and are therefore used with varying success to treat some types of cancer.

Therefore, in some embodiments, compositions are used to treat cancer. The gene modification technology can be designed to reduce or prevent expression of PD-1, and administered in an effective amount to do so.

The compositions can be used as antiviral agents, for example, when designed to modify a specific a portion of a viral genome necessary for proper proliferation or function of the virus.

Thus, for example, in some embodiments, the fetus or embryo has a genetic mutation that causes hemophilia, a hemoglobinopaty, cystic fibrosis, xeroderma, pigmentosum, or a lysosomal storage disease, and the methods include administering an effective amount of a gene editing composition to correct to the mutation in the fetus or embryo.

### Variants, Substitutions, and Exemplary PNAs

Preferred diseases and sequences of exemplary targeting sites, triplex forming molecules, and donor oligonucleotides are discussed in more detail below. Any of the sequences can also be modified as disclosed herein or otherwise known in the art. For example, in some embodiments, any of the triplex-forming molecules herein can have one or more mutations (e.g., substitutions, deletions, or insertions), such that the triplex-forming molecules still bind to the target sequence.

Any of the triplex-forming molecules herein can be manufactured using canonical nucleic acids or other suitable substitutes including those disclosed herein (e.g., PNAs), without or without any of the base, sugar, or backbone modifications discussed herein or in WO 1996/040271, WO/2010/123983, and U.S. Pat. No. 8,658,608.

Any of the triplex-forming molecules herein can be peptide nucleic acids. In some embodiments, one or more of the cytosines of any of triplex-forming molecules herein is substituted with a pseudoisocytosine. In some embodiments, all of the cytosines in the Hoogsteen-binding portion of a triplex forming molecule are substituted with pseudoisocytosine. In some embodiments, any of the triplex-forming molecules herein, includes one or more of peptide nucleic acid residues substituted with side chain (for example: amino acid side chain or miniPEG side chain) at the alpha, beta and/or gamma position of the backbone. For example, the PNA oligomer can comprise at least one residue comprising a gamma modification/substitution of a backbone carbon atom. In some embodiments all of the peptide nucleic acid residues in the Hoogsteen-binding portion only, the Watson-Crick-binding portion only, or across the entire PNA are substituted with γPNA residues. In particular embodiments, alternating residues are PNA and γPNA in the Hoogsteen-binding portion only, the Watson-Crick-binding portion only, or across the entire PNA are substituted. In some embodiments, the modifications are miniPEG γPNA residues, methyl γPNA residues, or another γ substitution discussed above. In some embodiments, the PNA oligomer includes two or more different modifications of the backbone (e.g. two different types of gamma side chains).

In some embodiments, (1) some or all of the residues in the Watson-Crick binding portion are γPNA residues (e.g., miniPEG-containing γPNA residues); (2) some or all of the residues in the Hoogsteen binding portion are γPNA residues (e.g., miniPEG-containing γPNA residues); or (3) some or all of the residues (in the Watson-Crick and/or Hoogsteen binding portions) are γPNA residues (e.g., miniPEG-containing γPNA residues). Therefore, in some embodiments any of the triplex forming molecules herein is a peptide nucleic acid wherein (1) all of the residues in the Watson-Crick binding portion are γPNA residues (e.g., miniPEG-containing γPNA residues) and none of the residues is in Hoogsteen binding portion are γPNA residues (e.g., miniPEG-containing γPNA residues); (2) all of the residues in the Hoogsteen binding portion are γPNA residues (e.g., miniPEG-containing γPNA residues) and none of the residues is in Watson-Crick binding portion are γPNA residues (e.g., miniPEG-containing γPNA residues); or (3) all of the residues (in the Watson-Crick and Hoogsteen binding portions) are γPNA residues (e.g., miniPEG-containing γPNA residues).

In some embodiments, the triplex-forming molecules are bis-peptide nucleic acids or tail-clamp PNAs with pseudoisocytosine substituted for one or more cytosines, particularly in the Hoogsteen-binding portion, and wherein some or all of the PNA residues are γPNA residues.

Any of the triplex-forming molecules herein can have one or more G-clamp-containing residues. For example, one or more cytosines or variant thereof such as pseudoisocytosine in any of the triplex-forming molecules herein can be substituted or otherwise modified to be a clamp-G (9-(2-guanidinoethoxy) phenoxazine).

Any of the triplex-forming molecules herein can include a flexible linker, linking, for example, a Hoogsteen-binding domain and a Watson-Crick binding domain to form a bis-PNA or tcPNA. The sequences can be linked with a flexible linker. For example, in some embodiments the flexible linker includes about 1-10, more preferably 2-5, most preferably about 3 units such as 8-amino-2, 6, 10-trioxaoctanoic acid residues. Some molecules include N-terminal or C-terminal non-binding residues, preferably positively charged residues. For example, some molecules include 1-10, preferably 2-5, most preferably about 3 lysines at the N-terminus, the C-terminus, or at both the N-terminus and the C-terminus.

For the disclosed sequences, “J” is pseudoisocytosine, “O” can be a flexible 8-amino-3,6-dioxaoctanoic acid, 6-aminohexanoic acid, or 8-amino-2, 6, 10-trioxaoctanoic acid moiety, “K” and “lys” (or “Lys”) are lysine.

PNA sequence are generally presented in N-terminal-to-C-terminal orientation.

In some embodiments, PNA sequences can be presented in the form: H-“nucleobase sequence”-NH₂ orientation, wherein the H represents the N-terminal hydrogen atom of an unmodified PNA oligomer and the —NH₂ represents the C-terminal amide of the polymer. For bis-PNA and tcPNA, the Hoosten-binding portion can be oriented up stream (e.g., at the “H” or N-terminal end of the polyamide) of the linker, while the Watson-Crick-binding portion can be oriented downstream (e.g., at the NH₂ (C-terminal) end) of the polymer/linker.

Any of the donor oligonucleotides can include optional phosphorothiate internucleoside linkages, particular between the two, three or four terminal 5′ and two, three or four terminal 3′ nucleotides. In some embodiments, the phosphorothioate internucleotide linkages need not be sequential and can be dispersed within the donor oligonucleotide. Nevertheless, the phosphorothioate internucleotide linkages can be oriented primarily near each termini of the donor oligonucleotide. Thus, each of the donor oligonucleotide sequences disclosed herein is expressly disclosed without any phosphorothiate internucleoside linkages, and with phosphorothiate internucleoside linkages, preferably between the two, three or four terminal 5′ and two, three or four terminal 3′ nucleotides.

### 1. Globinopathies

Worldwide, globinopathies account for significant morbidity and mortality. Over 1,200 different known genetic mutations affect the DNA sequence of the human alpha-like (HBZ, HBA2, HBA1, and HBQ1) and beta-like (HBE1, HBG1, HBD, and HBB) globin genes. Two of the more prevalent and well-studied globinopathies are sickle cell anemia and β-thalassemia. Substitution of valine for glutamic acid at position 6 of the β-globin chain in patients with sickle cell anemia predisposes to hemoglobin polymerization, leading to sickle cell rigidity and vasoocclusion with resulting tissue and organ damage. In patients with β-thalassemia, a variety of mutational mechanisms results in reduced synthesis of β-globin leading to accumulation of aggregates of unpaired, insoluble α-chains that cause ineffective erythropoiesis, accelerated red cell destruction, and severe anemia.

Together, globinopathies represent the most common single-gene disorders in man. Triplex forming molecules are particularly well suited to treat globinopathies, as they are single gene disorders caused by point mutations. Triplex forming molecules are effective at binding to the human β-globin both in vitro and in living cells, both ex vivo and in vivo (including by in utero application) in animals. Experimental results also demonstrate correction of a thalassemia-associated mutation in vivo in a transgenic mouse carrying a human beta globin gene with the IVS2-654 thalassemia mutation (in place of the endogenous mouse beta globin) with correction of the mutation in 4% of the total bone marrow cells, cure of the anemia with blood hemoglobin levels showing a sustained elevation into the normal range, reversal of extramedullary hematopoiesis and reversal of splenomegaly, and reduction in reticulocyte counts, following systemic administration of PNA and DNA containing nanoparticles.

β-thalassemia is an unstable hemoglobinopathy leading to the precipitation of α-hemoglobin within RBCs resulting in a severe hemolytic anemia. Patients experience jaundice and splenomegaly, with substantially decreased blood hemoglobin concentrations necessitating repeated transfusions, typically resulting in severe iron overload with time. Cardiac failure due to myocardial siderosis is a major cause of death from β-thalassemia by the end of the third decade. Reduction of repeated blood transfusions in these patients is therefore of primary importance to improve patient outcomes.

a. Exemplary β-Globin Gene Target Sites

In the β-globin gene sequence, particularly in the introns, there are many good third-strand binding sites that may be utilized in the methods disclosed herein. A portion of the GenBank sequence of the chromosome-11 human-native hemoglobin-gene cluster (GenBank: U01317.1—Human beta globin region on chromosome 11—LOCUS HUVMHBB, 73308 bp ds-DNA) from base 60001 to base 66060 is presented below. The start of the gene coding sequence at position 62187-62189 is indicated by wave underlining. This portion of the GenBank sequence contains the native β globin gene sequence. In sickle cell hemoglobin the adenine base at position 62206 is mutated to a thymine. Other common point mutations occur in intron 2 (IVS2). Mutations include IVS2-1, IVS2-566, IVS2-654, IVS2-705, and IVS2-745.

Exemplary triplex forming molecule binding sites, are provided in, for example, WO 1996/040271, WO/2010/123983, and U.S. Pat. No. 8,658,608, and in the working Examples below. Target regions can be reference based on the coding strand of genomic DNA, or the complementary non-coding sequence thereto (e.g., the Watson or Crick stand). Exemplary target regions are identified with reference to the coding sequence of the β globin gene sequence in the sequence below by double underlining and a combination of underlining and double underlining (wherein the underlining is optional additional binding sequence). Additionally, for each targeting sequence identified, the complementary target sequence on the reverse non-coding strand is also explicitly disclosed as a triplex forming molecule binding sequence.

Accordingly, triplex forming molecules can be designed to bind a target region on either the coding or non-coding strand. However, as discussed above, triplex-forming molecules, such as PNA and tcPNA preferably invade the target duplex, displacement of the polypyrimidine, and induce triplex formation with the displaced polypurine.

b. Exemplary Triplex Forming Sequences
i. Beta Thalassemia

Gene editing molecules can be designed based on the guidance provided herein and otherwise known in the art. Exemplary triplex forming molecule and donor sequences, are provided in, for example, WO 1996/040271, WO/2010/123983, and U.S. Pat. No. 8,658,608, and in the working Examples below, and can be altered to include one or more of the modifications disclosed herein.

Triplex forming molecules can include a sequence substantially complementary to the polypurine strand of the polypyrimidine:polypurine target motif.

### 2. Cystic Fibrosis

The disclosed compositions and methods can be used to treat cystic fibrosis. Cystic fibrosis (CF) is a lethal autosomal recessive disease caused by defects in the cystic fibrosis transmembrane conductance regulator (CFTR), an ion channel that mediates Cl-transport. Lack of CFTR function results in chronic obstructive lung disease and premature death due to respiratory failure, intestinal obstruction syndromes, exocrine and endocrine pancreatic dysfunction, and infertility (Davis, et al., Pediatr Rev., 22(8):257-64 (2001)). The most common mutation in CF is a three base-pair deletion (F508del) resulting in the loss of a phenylalanine residue, causing intracellular degradation of the CFTR protein and lack of cell surface expression (Davis, et al., Am J Respir Crit Care Med., 173(5):475-82 (2006)). In addition to this common mutation there are many other mutations that occur and lead to disease including a class of mutations due to premature stop codons, nonsense mutations. In fact nonsense mutations account for approximately 10% of disease causing mutations. Of the nonsense mutations G542X and W1282X are the most common with frequencies of 2.6% and 1.6% respectfully.

Although CF is one of the most rigorously characterized genetic diseases, current treatment of patients with CF focuses on symptomatic management rather than primary correction of the genetic defect. Gene therapy has remained an elusive target in CF, because of challenges of in vivo delivery to the lung and other organ systems (Armstrong, et al., Archives of disease in childhood (2014) doi: 10.1136/archdischild-2012-302158. PubMed PMID: 24464978). In recent years, there have been many advances in gene therapy for treatment of diseases involving the hematolymphoid system, where harvest and ex vivo manipulation of cells for autologous transplantation is possible: some examples include the use of zinc finger nucleases targeting CCR5 to produce HIV-1 resistant cells (Holt, et al., Nature biotechnology, 28(8):839-47 (2010)) correction of the ABCD1 gene by lentiviral vectors for treatment of adrenoleukodystrophy (Cartier, et al., Science, 326(5954):818-23 (2009)) and correction of SCID due to ADA deficiency using retroviral gene transfer (Aiuti, et al., The New England Journal Of Medicine, 360(5):447-58 (2009).

Harvest and autologous transplant is not an option in CF, due to the involvement of the lung and other internal organs. As one approach, the UK Cystic Fibrosis Gene Therapy Consortium has tested liposomes to deliver plasmids containing cDNA encoding CFTR to the lung (Alton, et al., Thorax, 68(11):1075-7 (2013)), Alton, et al., The Lancet Respiratory Medicine, (2015). doi: 10.1016/S2213-2600(15)00245-3. PubMed PMID: 26149841.) other clinical trials have used viral vectors for delivery of the CFTR gene or CFTR expression plasmids that are compacted by polyethylene glycol-substituted lysine 30-mer peptides with limited success (Konstan, et al., Human Gene Therapy, 15(12):1255-69 (2004)). Moreover, delivery of plasmid DNA for gene addition without targeted insertion does not result in correction of the endogenous gene and is not subject to normal CFTR gene regulation, and virus-mediated integration of the CFTR cDNA could introduce the risk of non-specific integration into important genomic sites.

However, it has been discovered that triplex-forming PNA molecules and donor DNA can be used to correct mutations leading to cystic fibrosis. In preferred embodiments, the compositions are administered by intranasal or pulmonary delivery. In some embodiments, the triplex-forming molecules can be administered in utero; for example by amniotic sac injection and/or injection into the vitelline vein. In utero approaches offer several advantages including, for example, the large number of somatic stem cells available for gene correction and a reduced inflammatory response due to the immune-privileged status of the fetus (see, e.g., Larson and Cohen, In Utero Gene Therapy, Ochsner J., 2(2):107-110 (2000)). Other exemplary advantages include stem cells are rapidly dividing, relatively smaller size of the organism compared to mature, adult organisms, a smaller dosage can be effective, therapies can be delivered before or during the pathogenesis of irreversible organ damage, etc.

In CF, for example, there is evidence of significant multisystem organ damage at birth

The compositions can be administered in an effective amount to induce or enhance gene correction in an amount effective to reduce one or more symptoms of cystic fibrosis. For example, in some embodiments, the gene correction occurs at an amount effective to improve impaired response to cyclic AMP stimulation, improve hyperpolarization in response to forskolin, reduction in the large lumen negative nasal potential, reduction in inflammatory cells in the bronchoalveolar lavage (BAL), improve lung histology, or a combination thereof. In some embodiments, the target cells are cells, particularly epithelial cells, that make up the sweat glands in the skin, that line passageways inside the lungs, liver, pancreas, or digestive or reproductive systems. In particular embodiments, the target cells are bronchial epithelial cells. While permanent genomic change using PNA/DNA is less transient than plasmid-based approaches and the changes will be passed on to daughter cells, some modified cells may be lost over time with regular turnover of the respiratory epithelium. In some embodiments, the target cells are lung epithelial progenitor cells. Modification of lung epithelial progenitors can induce more long-term correction of phenotype.

Sequences for the human cystic fibrosis transmembrane conductance regulator (CFTR) are known in the art, see, for example, GenBank Accession number: AH006034.1, and compositions and methods of targeted correction of CFTR are described in McNeer, et al., Nature Communications, 6:6952, (DOI 10.1038/ncomms7952), 11 pages.

### 3. Hiv

The gene editing compositions can be used to treat infections, for example those caused by HIV.

a. Exemplary Target Sites

The target sequence for the triplex-forming molecules is within or adjacent to a human gene that encodes a cell surface receptor for human immunodeficiency virus (HIV). Preferably, the target sequence of the triplex-forming molecules is within or is adjacent to a portion of a HIV receptor gene important to its function in HIV entry into cells, such as sequences that are involved in efficient expression of the receptor, transport of the receptor to the cell surface, stability of the receptor, viral binding by the receptor, or endocytosis of the receptor. Target sequences can be within the coding DNA sequence of the gene or within introns. Target sequences can also be within DNA sequences that regulate expression of the target gene, including promoter or enhancer sequences.

The target sequence can be within or adjacent to any gene encoding a cell surface receptor that facilitates entry of HIV into cells. The molecular mechanism of HIV entry into cells involves specific interactions between the viral envelope glycoproteins (env) and two target cell proteins, CD4 and the chemokine receptors. HIV cell tropism is determined by the specificity of the env for a particular chemokine receptor, a 7 transmembrane-spanning, G protein-coupled receptor (Steinberger, et al., Proc. Natl. Acad. Sci. USA. 97: 805-10 (2000)). The two major families of chemokine receptors are the CXC chemokine receptors and the CC chemokine receptors (CCR) so named for their binding of CXC and CC chemokines, respectively. While CXC chemokine receptors traditionally have been associated with acute inflammatory responses, the CCRs are mostly expressed on cell types found in connection with chronic inflammation and T-cell-mediated inflammatory reactions: eosinophils, basophils, monocytes, macrophages, dendritic cells, and T cells (Nansen, et al. 2002, Blood 99:4). In one embodiment, the target sequence is within or adjacent to the human genes encoding chemokine receptors, including, but not limited to, CXCR4, CCR5, CCR2b, CCR3, and CCR1.

In a preferred embodiment, the target sequence is within or adjacent to the human CCR5 gene. The CCR5 chemokine receptor is the major co-receptor for R5-tropic HIV strains, which are responsible for most cases of initial, acute HIV infection. Individuals who possess a homozygous inactivating mutation, referred to as the Δ32 mutation, in the CCR5 gene are almost completely resistant to infection by R5-tropic HIV-1 strains. The Δ32 mutation produces a 32 base pair deletion in the CCR5 coding region.

Another naturally occurring mutation in the CCR5 gene is the m303 mutation, characterized by an open reading frame single T to A base pair transversion at nucleotide 303 which indicates a cysteine to stop codon change in the first extracellular loop of the chemokine receptor protein at amino acid 101 (C101X) (Carrington et al. 1997). Mutagenesis assays have not detected the expression of the m303 co-receptor on the surface of CCR5 null transfected cells which were found to be non-susceptible to HIV-1 R5-isolates in infection assays (Blanpain, et al. (2000).

Compositions and methods for targeted gene therapy using triplex-forming oligonucleotides and peptide nucleic acids for treating infectious diseases such as HIV are described in U.S. Application No. 2008/050920 and WO 2011/133803. Each provides sequences of triplex forming molecules, target sequences, and donor oligonucleotides that can be utilized in the compositions and methods provided herein.

For example, individuals having the homozygous Δ32 inactivating mutation in the CCR5 gene display no significant adverse phenotypes, suggesting that this gene is largely dispensable for normal human health. This makes the CCR5 gene a particularly attractive target for targeted mutagenesis using the triplex-forming molecules disclosed herein. The gene for human CCR5 is known in the art and is provided at GENBANK accession number NM_000579. The coding region of the human CCR5 gene is provided by nucleotides 358 to 1416 of GENBANK accession number NM_000579.

In some embodiments, the target region is a polypurine site within or adjacent to a gene encoding a chemokine receptor including CXCR4, CCR5, CCR2b, CCR3, and CCR1. In a preferred embodiment, the target region is a polypurine or homopurine site within the coding region of the human CCR5 gene. Three homopurine sites in the coding region of the CCR5 gene that are especially useful as target sites for triplex-forming molecules are from positions 509-518, 679-690 and 900-908 relative to the ATG start codon. The homopurine site from 679-690 partially encompasses the site of the nonsense mutation created by the Δ32 mutation. Triplex-forming molecules that bind to this target site are particularly useful.

### 4. Lysosomal Storage Diseases

The compositions and methods compositions can also be used to treat lysosomal storage diseases. Lysosomal storage diseases (LSDs) are a group of more than 50 clinically-recognized, rare inherited metabolic disorders that result from defects in lysosomal function (Walkley, J. Inherit. Metab. Dis., 32(2):181-9 (2009)). Lysosomal storage disorders are caused by dysfunction of the cell's lysosome orangelle, which is part of the larger endosomal/lysosomal system. Together with the ubiquitin-proteosomal and autophagosomal systems, the lysosome is essential to substrate degradation and recycling, homeostatic control, and signaling within the cell. Lysosomal dysfunction is usually the result of a deficiency of a single enzyme necessary for the metabolism of lipids, glycoproteins (sugar containing proteins) or mucopolysaccharides (long unbranched polysaccharides consisting of a repeating disaccharide unit; also known as glycosaminoglycans, or GAGs) which are fated for breakdown or recycling. Enzyme deficiency reduces or prevents break down or recycling of the unwanted lipids, glycoproteins, and GAGs, and results in buildup or “storage” of these materials within the cell. Most lysosomal diseases show widespread tissue and organ involvement, with brain, viscera, bone and connective tissues often being affected. More than two-thirds of lysosomal diseases affect the brain. Neurons appear particularly vulnerable to lysosomal dysfunction, exhibiting a range of defects from specific axonal and dendritic abnormalities to neuron death.

Individually, LSDs occur with incidences of less than 1:100,000, however, as a group the incidence is as high as 1 in 1,500 to 7,000 live births (Staretz-Chacham, et al., Pediatrics, 123(4):1191-207 (2009)). LSDs are typically the result of inborn genetic errors. Most of these disorders are autosomal recessively inherited, however a few are X-linked recessively inherited, such as Fabry disease and Hunter syndrome (MPS II). Affected individuals generally appear normal at birth, however the diseases are progressive. Develop of clinical disease may not occur until years or decades later, but is typically fatal. Lysosomal storage diseases affect mostly children and they often die at a young and unpredictable age, many within a few months or years of birth. This makes these types of lysosomal storage diseases attractive for pre-natal intervention. Many other children die of this disease following years of suffering from various symptoms of their particular disorder. Clinical disease may be manifest as mental retardation and/or dementia, sensory loss including blindness or deafness, motor system dysfunction, seizures, sleep and behavioral disturbances, and so forth. Some people with Lysosomal storage disease have enlarged livers (hepatomegaly) and enlarged spleens (splenomegaly), pulmonary and cardiac problems, and bones that grow abnormally.

Treatment for many LSDs is enzyme replacement therapy (ERT) and/or substrate reduction therapy (SRT), as wells as treatment or management of symptoms. The average annual cost of ERT in the United States ranges from $90,000 to $565,000. While ERT has significant systemic clinical efficacy for a variety of LSDs, little or no effects are seen on central nervous system (CNS) disease symptoms, because the recombinant proteins cannot penetrate the blood-brain barrier. Allogeneic hematopoietic stem cell transplantation (HSCT) represents a highly effective treatment for selected LSDs. It is currently the only means to prevent the progression of associated neurologic sequelae. However, HSCT is expensive, requires an HLA-matched donor and is associated with significant morbidity and mortality. Recent gene therapy studies suggest that LSDs are good targets for this type of treatment.

Compositions and methods for targeted gene therapy using triplex-forming oligonucleotides and peptide nucleic acids for treating lysosomal storage diseases are described in WO 2011/133802, which provides sequences of triplex forming molecules, target sequences, and donor oligonucleotides that can be utilized in the compositions and methods provided herein.

For example, the compositions and methods can be are employed to treat Gaucher's disease (GD). Gaucher's disease, also known as Gaucher syndrome, is the most common lysosomal storage disease. Gaucher's disease is an inherited genetic disease in which lipid accumulates in cells and certain organs due to deficiency of the enzyme glucocerebrosidase (also known as acid β-glucosidase) in lysosomes. Glucocerebrosidase enzyme contributes to the degradation of the fatty substance glucocerebroside (also known as glucosylceramide) by cleaving b-glycoside into b-glucose and ceramide residues (Scriver C R, Beaudet A L, Valle D, Sly W S. The metabolic and molecular basis of inherited disease. 8th ed. New York: McGraw-Hill Pub, 2001: 3635-3668). When the enzyme is defective, the substance accumulates, particularly in cells of the mononuclear cell lineage, and organs and tissues including the spleen, liver, kidneys, lungs, brain and bone marrow.

There are two major forms: non-neuropathic (type 1, most commonly observed type in adulthood) and neuropathic (type 2 and 3). GBA (GBA glucosidase, beta, acid), the only known human gene responsible for glucosidase-mediated GD, is located on chromosome 1, location 1q21. More than 200 mutations have been defined within the known genomic sequence of this single gene (NCBI Reference Sequence: NG 009783.1). The most commonly observed mutations are N370S, L444P, RecNciI, 84GG, R463C, recTL and 84 GG is a null mutation in which there is no capacity to synthesize enzyme. However, N370S mutation is almost always related with type 1 disease and milder forms of disease. Very rarely, deficiency of sphingolipid activator protein (Gaucher factor, SAP-2, saposin C) may result in GD. In some embodiments, triplex-forming molecules are used to induce recombination of donor oligonucleotides designed to correct mutations in GBA.

In another embodiment, compositions and the methods herein are used to treat Fabry disease (also known as Fabry's disease, Anderson-Fabry disease, angiokeratoma corporis diffusum and alpha-galactosidase A deficiency), a rare X-linked recessive disordered, resulting from a deficiency of the enzyme alpha galactosidase A (a-GAL A, encoded by GLA). The human gene encoding GLA has a known genomic sequence (NCBI Reference Sequence: NG_007119.1) and is located at Xp22 of the X chromosome. Mutations in GLA result in accumulation of the glycolipid globotriaosylceramide (abbreviated as Gb3, GL-3, or ceramide trihexoside) within the blood vessels, other tissues, and organs, resulting in impairment of their proper function (Karen, et al., Dermatol. Online J., 11 (4): 8 (2005)). The condition affects hemizygous males (i.e. all males), as well as homozygous, and potentially heterozygous (carrier), females. Males typically experience severe symptoms, while women can range from being asymptomatic to having severe symptoms. This variability is thought to be due to X-inactivation patterns during embryonic development of the female. In some embodiments, triplex-forming molecules are used to induce recombination of donor oligonucleotides designed to correct mutations in GLA.

In preferred embodiments, the compositions and methods are used to treat Hurler syndrome (HS). Hurler syndrome, also known as mucopolysaccharidosis type I (MPS I), α-L-iduronidase deficiency, and Hurler's disease, is a genetic disorder that results in the buildup of mucopolysaccharides due to a deficiency of α-L iduronidase, an enzyme responsible for the degradation of mucopolysaccharides in lysosomes (Dib and Pastories, Genet. Mol. Res., 6(3):667-74 (2007)). MPS I is divided into three subtypes based on severity of symptoms. All three types result from an absence of, or insufficient levels of, the enzyme α-L-iduronidase. MPS I H or Hurler syndrome is the most severe of the MPS I subtypes. The other two types are MPS I S or Scheie syndrome and MPS I H-S or Hurler-Scheie syndrome. Without α-L-iduronidase, heparan sulfate and dermatan sulfate, the main components of connective tissues, build-up in the body. Excessive amounts of glycosaminoglycans (GAGs) pass into the blood circulation and are stored throughout the body, with some excreted in the urine. Symptoms appear during childhood, and can include developmental delay as early as the first year of age. Patients usually reach a plateau in their development between the ages of two and four years, followed by progressive mental decline and loss of physical skills (Scott et al., Hum. Mutat. 6: 288-302 (1995)). Language may be limited due to hearing loss and an enlarged tongue, and eventually site impairment can result from clouding of cornea and retinal degeneration. Carpal tunnel syndrome (or similar compression of nerves elsewhere in the body) and restricted joint movement are also common.

### XI. Combination Therapies

Each of the different active agents including components of gene editing and potentiation here can be administered alone or in any combination and further in combination with one or more additional active agents. In all cases, the combination of agents can be part of the same admixture, or administered as separate compositions. In some embodiments, the separate compositions are administered through the same route of administration. In other embodiments, the separate compositions are administered through different routes of administration.

### A. Conventional Therapeutic Agents

Examples of preferred additional active agents include other conventional therapies known in the art for treating the desired disease or condition. For example, in the treatment of sickle cell disease, the additional therapy may be hydroxurea.

In the treatment of cystic fibrosis, the additional therapy may include mucolytics, antibiotics, nutritional agents, etc. Specific drugs are outlined in the Cystic Fibrosis Foundation drug pipeline and include, but are not limited to, CFTR modulators such as KALYDECO® (invascaftor), ORKAMBI™ (lumacaftor+ivacaftor), ataluren (PTC124), VX-661+invacaftor, riociguat, QBW251, N91115, and QR-010; agents that improve airway surface liquid such as hypertonic saline, bronchitol, and P-1037; mucus alteration agents such as PULMOZYME® (dornase alfa); anti-inflammatories such as ibuprofen, alpha 1 anti-trypsin, CTX-4430, and JBT-101; anti-infective such as inhaled tobramycin, azithromycin, CAYSTON® (aztreonam for inhalation solution), TOBI inhaled powder, levofloxacin, ARIKACE® (nebulized liposomal amikacin), AEROVANC® (vancomycin hydrochloride inhalation powder), and gallium; and nutritional supplements such as aquADEKs, pancrelipase enzyme products, liprotamase, and burlulipase.

In the treatment of HIV, the additional therapy maybe an antiretroviral agents including, but not limited to, a non-nucleoside reverse transcriptase inhibitor (NNRTIs), a nucleoside reverse transcriptase inhibitor (NRTIs), a protease inhibitors (PIs), a fusion inhibitors, a CCR5 antagonists (CCR5s) (also called entry inhibitors), an integrase strand transfer inhibitors (INSTIs), or a combination thereof.

In the treatment of lysosomal storage disease, the additional therapy could include, for example, enzyme replacement therapy, bone marrow transplantation, or a combination thereof.

### B. Additional Mutagenic Agents

The compositions can be used in combination with other mutagenic agents. In a preferred embodiment, the additional mutagenic agents are conjugated or linked to gene editing technology or a delivery vehicle (such as a nanoparticle or microparticle) thereof. Additional mutagenic agents that can be used in combination with gene editing technology, particularly triplex forming molecules, include agents that are capable of directing mutagenesis, nucleic acid crosslinkers, radioactive agents, or alkylating groups, or molecules that can recruit DNA-damaging cellular enzymes. Other suitable mutagenic agents include, but are not limited to, chemical mutagenic agents such as alkylating, bialkylating or intercalating agents. A preferred agent for co-administration is psoralen-linked molecules as described in PCT/US/94/07234 by Yale University.

It may also be desirable to administer gene editing compositions in combination with agents that further enhance the frequency of gene modification in cells. For example, the compositions can be administered in combination with a histone deacetylase (HDAC) inhibitor, such as suberoylanilide hydroxamic acid (SAHA), which has been found to promote increased levels of gene targeting in asynchronous cells.

The nucleotide excision repair pathway is also known to facilitate triplex-forming molecule-mediated recombination. Therefore, the compositions can be administered in combination with an agent that enhances or increases the nucleotide excision repair pathway, for example an agent that increases the expression, or activity, or localization to the target site, of the endogenous damage recognition factor XPA.

Compositions may also be administered in combination with a second active agent that enhances uptake or delivery of the gene editing technology. For example, the lysosomotropic agent chloroquine has been shown to enhance delivery of PNAs into cells (Abes, et al., J C ontroll. Rel., 110:595-604 (2006). Agents that improve the frequency of gene modification are particularly useful for in vitro and ex vivo application, for example ex vivo modification of hematopoietic stem cells for therapeutic use.

### XII. Methods for Determining Triplex Formation and Gene Modification

### A. Methods for Determining Triplex Formation

A useful measure of triple helix formation is the equilibrium dissociation constant, K_{d}, of the triplex, which can be estimated as the concentration of triplex-forming molecules at which triplex formation is half-maximal. Preferably, the molecules have a binding affinity for the target sequence in the range of physiologic interactions. Preferred triplex-forming molecules have a K_{d} less than or equal to approximately 10⁻⁷ M. Most preferably, the K_{d} is less than or equal to 2×10⁻⁸ M in order to achieve significant intramolecular interactions. A variety of methods are available to determine the K_{d} of triplex-forming molecules with the target duplex. In the examples which follow, the K_{d} was estimated using a gel mobility shift assay (R. H. Durland et al., Biochemistry 30, 9246 (1991)). The dissociation constant (K_{d}) can be determined as the concentration of triplex-forming molecules in which half was bound to the target sequence and half was unbound.

### B. Methods for Determining Gene Modification

Sequencing and allele-specific PCR are preferred methods for determining if gene modification has occurred. PCR primers are designed to distinguish between the original allele, and the new predicted sequence following recombination. Other methods of determining if a recombination event has occurred are known in the art and may be selected based on the type of modification made. Methods include, but are not limited to, analysis of genomic DNA, for example by sequencing, allele-specific PCR, or restriction endonuclease selective PCR (REMS-PCR); analysis of mRNA transcribed from the target gene for example by Northern blot, in situ hybridization, real-time or quantitative reverse transcriptase (RT) PCT; and analysis of the polypeptide encoded by the target gene, for example, by immunostaining, ELISA, or FACS. In some cases, modified cells will be compared to parental controls. Other methods may include testing for changes in the function of the RNA transcribed by, or the polypeptide encoded by the target gene. For example, if the target gene encodes an enzyme, an assay designed to test enzyme function may be used.

### XIII. Kits

Medical kits are also disclosed. The medical kits can include, for example, a dosage supply of gene editing technology or a potentiating agent thereof, or a combination thereof in separately or together in the same admixture. The active agents can be supplied alone (e.g., lyophilized), or in a pharmaceutical composition. The active agents can be in a unit dosage, or in a stock that should be diluted prior to administration. In some embodiments, the kit includes a supply of pharmaceutically acceptable carrier. The kit can also include devices for administration of the active agents or compositions, for example, syringes. The kits can include printed instructions for administering the compound in a use as described above.

### EXAMPLES

### Example 1: Triplex-Forming PNA Design and Nanoparticle Formulation for Gene Editing of a β-Globin Mutation

### Materials and Methods

### Oligonucleotides

^{MP}γPNA monomers were prepared as reported (Sahu, et al., J. Org. Chem., 76:5614-5627 (2011)). PNA oligomers were synthesized on solid support using Boc chemistry, as described (Bahal, et al., ChemBioChem, 13:56-60 (2012)). The sequences of PNAs used in this study are:

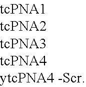

Sequences of tcPNAs and γtcPNAs used in this study to bind to positions 577 to 595 (tcPNA1 and γtcPNA4), 611 to 629 (tcPNA2), and 807 to 825 (tcPNA3) in β-globin intron 2 within the β-globin/GFP fusion gene and within the human β-globin gene in the thalassemic mouse model. γtcPNA4-Scr is a scrambled version of γtcPNA4 with the same base composition.

The single-stranded donor DNA oligomer was prepared by standard DNA synthesis except for the inclusion of 3 phosphorothiate internucleoside linkages at each end to protect from nuclease degradation. The sequence of the donor DNA matches positions 624 to 684 in β-globin intron 2.

### PLGA Nanoparticle Synthesis and Characterization

PLGA nanoparticles containing the PNAs and DNAs were formulated using a double-emulsion solvent evaporation method and characterized as previously described (McNeer, et al., Molecular Therapy, 19(1):172-180 (2011), and). Release profiles were analyzed as previously described (McNeer, et al., Mol. Ther., 19:172-180 (2011)).

### DNA Binding Gel Shift Assays

For gel electrophoresis, synthetic 120 bp dsDNA targets were incubated with indicated oligomers at 37 C in low ionic strength buffer (10 mM NaPi, pH 7.4). The samples were separated on 10% non-denaturing polyacrylamide gels in 1×TBE buffer. The gels were run at 100 V/cm for 1.5 hr. After electrophoresis, the gels were stained with 1×SYBR-Gold (catalog #S11494, Invitrogen) for 10 min, washed 2× with 1×TBE buffer, and then imaged using a gel documentation system (BioDoc-It System). The images were then inverted using Adobe Photoshop 6.0.

### Results

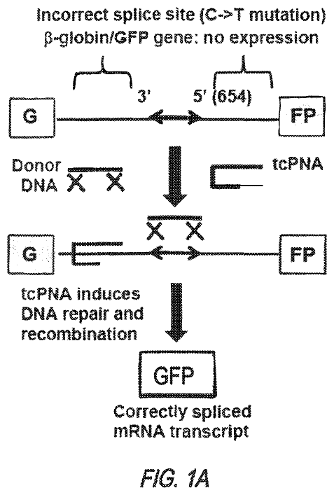

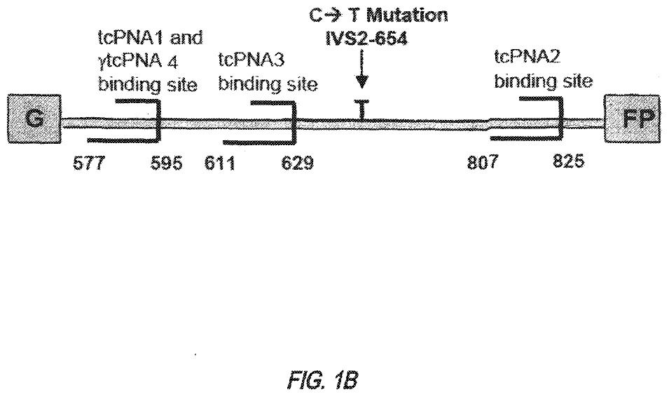

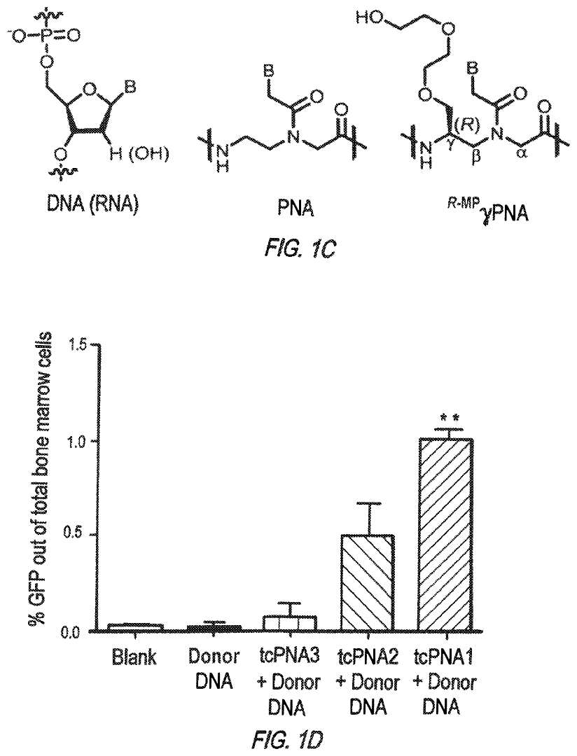

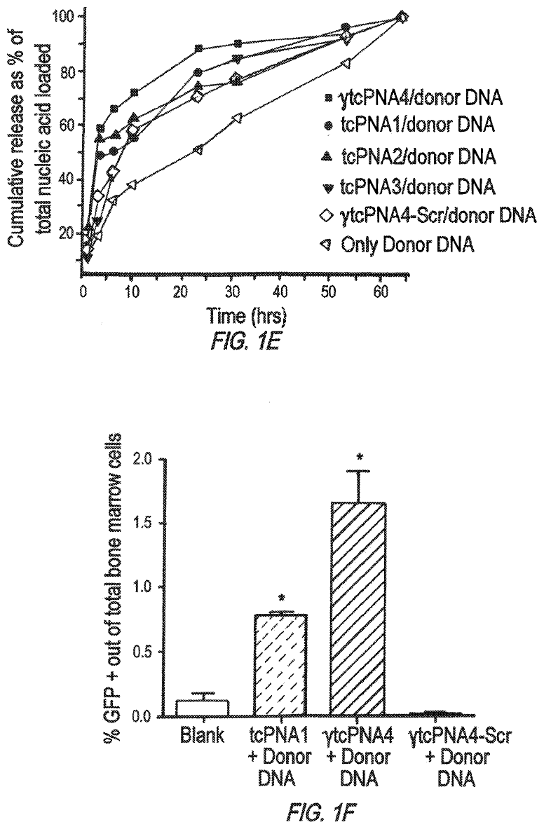

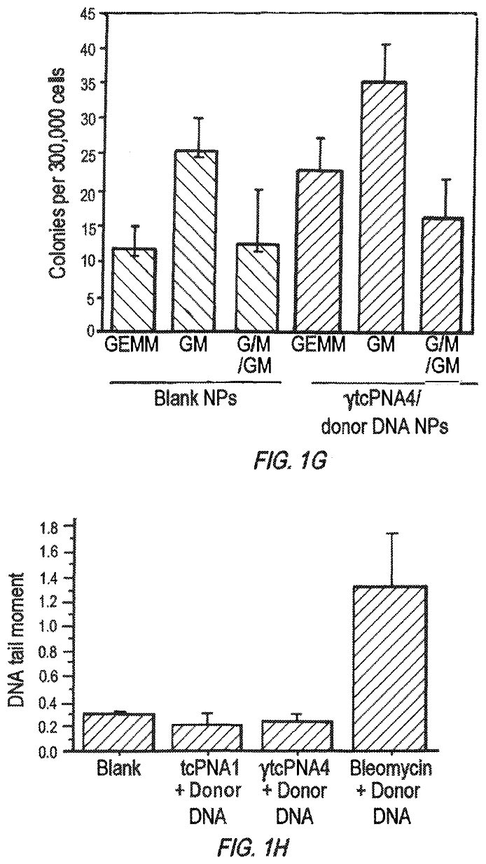

To assay for gene editing in a robust and quantitative manner, a transgenic mouse model was utilized with a β-globin/GFP fusion transgene of human β-globin intron 2 carrying a thalassemia-associated IVS2-654 (C-T) mutation embedded within the GFP coding sequence, resulting in incorrect splicing of β-globin/GFP mRNA and lack of GFP expression (Sazani, et al., Nat. Biotechnol., 20:1228-1233 (2002)). PNA-mediated triplex-formation induces DNA repair and recombination of the genomic site with a 60-nucleotide sense donor DNA that is homologous to a portion of the β-globin intron 2 sequence except for providing a wild-type nucleotide at the IVS2-654 position. Via recombination, the splice-site mutation is corrected and expression of functional GFP occurs (FIG. 1A) (McNeer, et al., Gene Therapy, 20:658-669 (2013); Bahal, et al., Curr. Gene Ther., 14:331-342 (2014)). Hence, GFP expression provides a direct phenotypic assessment of genome editing frequencies that can be quantified by flow cytometry.

A series of tcPNAs were designed to bind to selected polypurine stretches in the β-globin intron in the vicinity of the IVS2-654 mutation. Two of the tcPNAs were synthesized to contain partial substitution with a mini-polyethylene-glycol (mini-PEG) group at the γ position (^{MP}γPNA) (FIG. 1B). Gamma substitutions in PNAs have been shown to enhance strand invasion and DNA binding affinity in the Watson-Crick binding mode due to helical pre-organization enforced by the modification (Bahal, et al., ChemBioChem, 13:56-60 (2012)). γtcPNA4 matches the sequence of tcPNA1 except that it contains γ units at alternating positions in the Watson-Crick domain (see sequences above). Scrambled γtcPNA (γtcPNA4-Scr) had the same base composition as γtcPNA4 but a scrambled sequence. All tcPNA oligomers were synthesized with 3 lysines at both termini to improve solubility and increase binding affinity to genomic DNA (see sequences above).

Gel shift assays to assess the binding of the tcPNAs to 120-bp DNA duplexes containing the respective target sequences showed that all of the tcPNAs bound specifically to their target sites in duplex DNA under physiological conditions. No binding was seen in the case of the scrambled sequence γtcPNA4-Scr oligomer.

Poly(lactic-co-glycolic acid) (PLGA) NPs can effectively deliver PNA/donor DNA combinations into primary human and mouse hematopoietic cells with essentially no toxicity (McNeer, et al., Gene Therapy, 20:658-669 (2013); Schleifman, et al., Mol. Ther.—Nucleic Acids, 2:e135 (2013); McNeer, et al., Mol. Ther., 19:172-180 (2011)). Here, tcPNAs and donor DNAs, at a molar ratio of 2:1, were incorporated into PLGA NPs. The NP formulations were evaluated by scanning electron microscopy (SEM) and dynamic light scattering (DLS). All the NPs exhibited sizes within the expected range and showed a uniform charge distribution as calculated from their zeta potential.

TABLE 1
Hydrodynamic diameter of formulated PLGA nanoparticles
measured using dynamic light scattering in PBS buffer.

| Sample | Diameter (nm) |
|---|---|
| tcPNA1/donar DNA | 293.1 ± 6.1 |
| tcPNA 2/donor DNA | 610.6 ± 27.7 |
| tcPNA 3/donor DNA | 373.0 ± 4.3 |
| γtcPNA 4/donor DNA | 291.0 ± 4.7 |
| γtcPNA 4-Scr/donor DNA | 458.6 ± 8.2 |
| Donor DNA | 907.3 ± 200 |

TABLE 2
Zeta potential of formulated PLGA nanoparticles.

| Sample | Zeta Potential (mV) |
|---|---|
| tcPNA1/donor DNA | −24.6 ± 0.4 |
| tcPNA2/donor DNA | −16.5 ± 0.5 |
| tcPNA3/donor DNA | −23.6 ± 0.5 |
| γtcPNA4/donor DNA | −23.4 ± 0.5 |
| γtcPNA 4-Scr/donor DNA | −19.5 ± 1.3 |
| Donor DNA | −29.1 ± 0.4 |

Nucleic acid release profiles in aqueous solution were consistent with previous studies, indicating no deleterious impact of the γ modifications on release from NPs (FIG. 1E).

### Example 2: γtcPNA Edit Bone Marrow Cell Genome Ex Vivo

### Materials and Methods

### Ex Vivo Experiments

Bone marrow cells were harvested by flushing of femurs and tibias from β-globin/GFP transgenic mice with Roswell Park Memorial Institute (RPMI)/10% FBS media. Two mg/ml of nanoparticles were used to treat approximately 300,000-500,000 cells for 48 hr in RPMI/10% FBS media containing glutamine, in triplicate samples. After 48 hr, cells were fixed by using 4% paraformaldehyde, and flow cytometry analyses were performed. Cells treated with blank nanoparticles were included as a control.

For CD117+ cell experiments, Iscove's Modified Dulbecco's Media (IMDM) media containing insulin (10 ng/ml), FCS (10%) and erythropoietin (1U/ml) was used to culture CD117+ cells after isolation using magnetic separation. Where indicated, 3 μg/ml of SCF (Recombinant murine SCF, catalog #250-03, PeproTech, Rocky Hill, NJ;) was added prior to nanoparticle treatment. 2 mg/ml of NPs were used to treat 50,000-100,000 CD117+ cells in triplicate for 48 hrs in the above media, followed by flow cytometry analyses as above. Inhibitors were used at concentrations of 200 nM (dasatinib), 1.0 μM (MEK162) and 3.0 μM (BKM120). Dasatanib was obtained from Cayman Chemical (Ann Arbor, MI; item #11498) and dissolved according to manufacturer's protocol. MEK162 and BKM120 were obtained from Dr. Harriet Kluger, Yale University.

### Comet Assay

400,000 bone marrow cells/well were plated on 6-well plates in 1 mL media, then treated with 2 mg/mL of PLGA nanoparticles with or without PNA and donor DNA. After 48 hours, cells were scraped and harvested, and prepared using the Trevigen Comet Assay kit per manufacturer's protocol (Trevigen, Gaithersburg, MD). Briefly, cells were suspended in agarose, added to comet slides, allowed to set, incubated 1 hr in lysis solution, placed in electrophoresis solution for 30 min, then run at 21 V for 45 min, placed in acetate solution for 30 min, transferred to 70% ethanol solution for 30 min, dried, stained with Sybr Green for 30 min, then visualized using an EVOS microscope. TriTek Comet Score freeware was used to analyze images.

### Results

Bone marrow cells harvested from β-globin/GFP transgenic mice were treated ex vivo with PLGA NPs containing tcPNA1/donor DNA, tcPNA2/donor DNA and tcPNA3/donor DNA combinations. After 48 hr, the percentage of GFP+(corrected) cells was quantified via flow cytometry, revealing that tcPNA1/donor DNA, tcPNA2/donor and tcPNA3/donor DNA-containing NPs induced genome modification at frequencies of ~1.0%, 0.51% and 0.1% respectively (FIG. 1D). The higher gene editing activity of tcPNA1 is likely due to its longer Hoogsteen binding domain, as previously observed (Schleifman, et al., Chem. Biol. (Cambridge, MA, U.S.), 18:1189-1198 (2011))). NPs containing the γ-substituted tcPNA (γtcPNA4) and donor DNA yielded significantly higher gene modification (1.62%) (FIG. 1F), showing that the ^{MP}γ substitutions confer increased biological activity that correlates with their improved binding properties. NPs with the γ-substituted but scrambled sequence γtcPNA4-Scr produced no modification (FIG. 1F).

Bone marrow cells treated with either blank NPs or NPs containing γtcPNA4/donor DNA were plated in methylcellulose medium supplemented with selected cytokines for growth of granulocyte/macrophage colonies (CFU-G, CFU-M and CFU-GM) or combined colonies (CFU-GEMM, granulocyte, erythroid, monocyte/macrophage, megakaryocyte). The two sets of treated cells formed myeloid and erythroid colonies at similar frequencies, indicating that treatment with γtcPNA4 and donor DNA does not impair the ability of the progenitor cells to proliferate and differentiate (FIG. 1G). Sequencing analysis of genomic DNA from selected GFP-positive methylcellulose colonies confirmed the presence of the targeted gene modification in the β-globin/GFP transgene at the IVS2-654 base pair. In other assays for toxicity, there was no increase in DNA double-strand breaks (DSBs) in the cells treated with γtcPNA4/donor DNA-containing NPs compared to blank NPs based on a single-cell gel electrophoresis assay (Comet assay) (FIG. 1H) and there was no induction of the inflammatory cytokines, TNF-alpha or interleukin-6 (IL-6), in the treated bone marrow cells, consistent with prior work with NPs containing standard PNAs (McNeer, et al., Gene Therapy, 20:658-669 (2013); Schleifman, et al., Mol. Ther.—Nucleic Acids, 2:e135 (2013); McNeer, et al., Mol. Ther., 19:172-180 (2011)).

### Example 3: Gene Modification is Elevated by γtcPNAs in CD117+ Hematopoietic Cells

### Materials and Methods

### Cell Sorting and Flow Cytometry

BD Bioscience kit catalog #558451 (BDImagTm Hematopoietic Progenitor Stem Cell Enrichment Set—DM) was used to isolate CD117 cells. Enrichment for CD117 was confirmed by flow cytometry. CD117+ enriched cells were labeled with CD117− APC (BD Pharmingen™ catalog #558451) antibody. Cells were co-labelled with control IgG antibody (BD Pharmingen™ catalog #555746) for gating purposes. To quantify GFP expression, after CD117 co-labelling, flow cytometry was performed using FACScaliburS by resuspending cells in PBS/1% FBS where green fluorescent cells are measured in the Fl1 channel and APC stained cells are in the F14 channel. Antibodies for other markers were Ter119 (BD Pharmingen™ catalog #561033) and CD45 APC (BD Pharmingen™ catalog #561018).

### Results

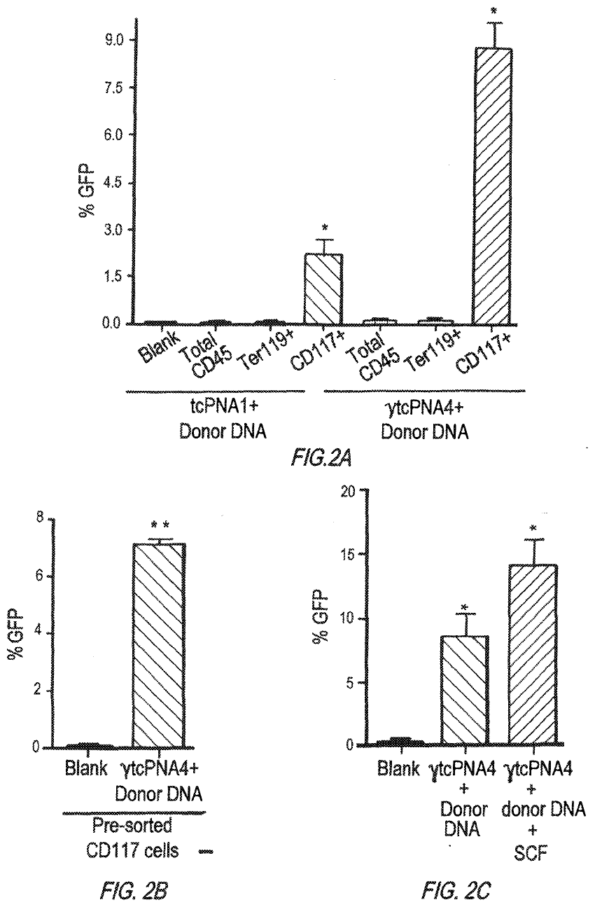

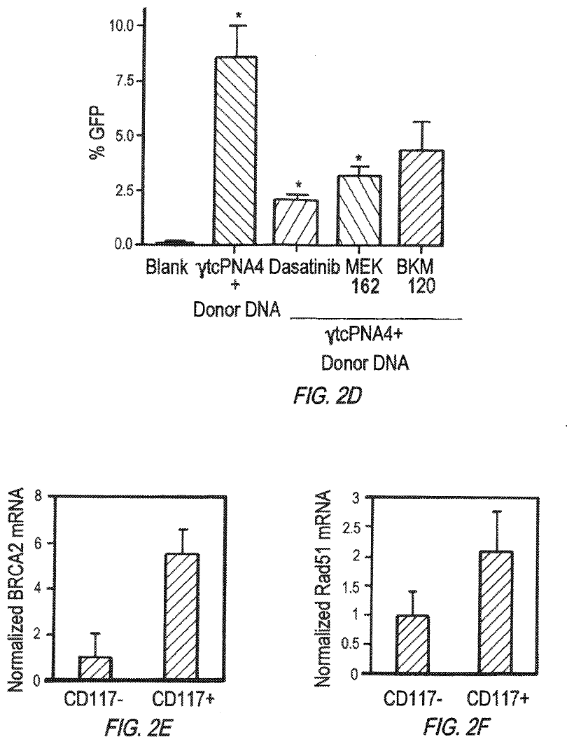

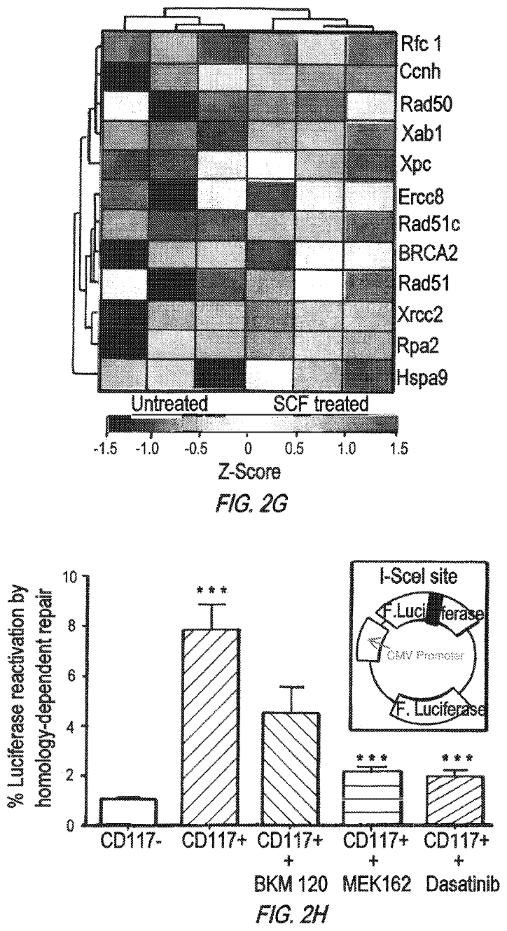

Previous work indicated that there might be increased activation of PNA-mediated DNA repair in certain colony-forming progenitors (McNeer, et al., Gene Therapy, 20:658-669 (2013)). To test this, whole bone marrow cells were treated with either blank NPs, NPs containing tcPNA1/donor DNA, or NPs containing γtcPNA4/donor DNA. Two days later, flow cytometry was performed to assess the frequency of GFP+ cells within selected sub-populations. Substantially elevated gene editing was observed in CD117+ cells compared to the total CD45+ cell population (FIG. 2A), with a frequency of 8.6% in CD117+ cells after a single treatment with the γtcPNA4/donor DNA NPs. The less potent tcPNA1/donor DNA NPs still yielded an elevated correction frequency of 2.1% in the CD117+ cells. The Ter119+ population, which includes more mature cells committed to the erythroid lineage, showed minimal susceptibility to gene editing with either PNA.

Next, the predisposition of CD117+ cells to increased gene editing was tested by first sorting for CD117+ cells prior to treatment with the NPs (FIG. 2B). An elevated percentage of modification (7.2%) was again seen in the CD117+ cells after a single treatment (FIG. 2B).

### Example 4: The c-Kit Pathway Mediates Increased Gene Modification in CD117+ Cells

CD117 (also known as mast/stem cell growth factor receptor or proto-oncogene c-Kit protein) is a receptor tyrosine kinase expressed on the surface of hematopoietic stem and progenitor cells as well as other cell types. Stem cell factor (SCF), the ligand for c-Kit, causes dimerization of the receptor and activates its tyrosine kinase activity to trigger downstream signaling pathways that can impact survival, proliferation, and differentiation.

To explore the mechanism of the increased gene editing in CD117+ cells, the requirement of c-Kit-dependent signaling for elevated gene correction or whether CD117 simply serves as a marker for the increased susceptibility to gene editing was distinguished. To do this, γtcPNA4/donor DNA NP-mediated gene editing was assayed in pre-sorted CD117+ cells in the presence or absence of selected kinase inhibitors (FIG. 2D). Dasatinib, which inhibits the c-Kit kinase in addition to the BCR/Abl and Src kinases, reduced the gene editing from 7% to 2.0%. Inhibitors of signaling factors downstream of c-Kit, including mitogen/extracellular signal-regulated kinase (MEK) (Binimetinib; MEK162) and phosphatidylinositol-3-kinase (PI3K) (BKM120), also decreased the gene editing frequencies in CD117+ cells to 2.6% and 4.1%, respectively (FIG. 2D).

On the other hand, when the CD117+ cells were treated with the c-Kit ligand, SCF, a significant increase in γtcPNA4/donor DNA-mediated gene editing (up to almost 15%) was observed (FIG. 2C). These results indicate that the SCF/c-Kit signaling can enhance gene editing and identify SCF as a potential agent to stimulate PNA-mediated gene editing.

### Example 5: Expression of DNA Repair Genes are Increased Upon Activation of the c-Kit+ Pathway

### Materials and Methods

### Microarray Analysis

Microarray analyses were performed on CD117+ and CD117− cells obtained from bone marrow of three separate β-globin/GFP mice at Yale Center of genomic analysis at Yale west campus. Each replicate cell sample was obtained from a separate mouse. RNA was extracted from 2×10⁶ for each sample using the RNeasy Mini Plus kit from Qiagen, as per the manufacturer's protocol. Following DNase treatment, total RNA was sequenced and analyzed at the Yale Center for Genome Analysis. Heat maps were generated using variance stabilizing transformations of the count data on the basis of a parametric fit to the overall mean dispersions.

### RT-PCR Analysis

Cells were harvested, pelleted, and stored frozen in RNA stabilization reagent (Qiagen), until ready for RNA extraction. RNA was extracted from the cell pellets using the RNAeasy Mini Plus kit from Qiagen, as per the manufacturer's protocol. The Invitrogen SuperScript III kit was used to generate cDNA from the RNA, as per the manufacturer's protocol, using 500 ng of RNA per reaction. PCR reactions contained cDNA, 20% Betaine, 0.2 mM dNTPS, Advantage 2 Polymerase Mix, 0.2 M of each primer, 2% Platinum Taq, and Brilliant SYBR Green. Primers and ROX reference dye were obtained from Stratagene and analysis was conducted using a Mx3000p realtime cycler. Cycler conditions were 94° C. for 2 min, 40 cycles of 94° C. 30 s/50° C. 30 s/72° C. 1 min, then 95° C. 1 min. Relative expression were calculated using the 2ΔΔCt method (Ct<36) and then normalized. For RAD51 analysis, Rad51 mRNA was quantified by using TaqMan® Gene Expression Assay (Life technologies, Mm00487905_ml) kit and using gene 18S (Life technologies, Mm03928990_g1) as a control.

### Western Blot Analysis

CD117+ and CD117− cells were isolated from β-globin/GFP mice and protein was extracted with Radio-Immunoprecipitation Assay (RIPA) lysis buffer. 50-100 μg total protein was run on SDS/PAGE gels and transferred to nitrocellulose membranes. Antibodies used were: Anti-BRCA2 (Ab-1) mouse mAb (EMD Millipore, OP95-100 ug) anti-RAD51-antibody (Santa Cruz biotechnology, SC 8349)).

### Results

The increased gene editing in the c-Kit+(CD117) cells was not explained by differential uptake of the NPs, as there were no detectable differences in uptake across several bone marrow cell sub-populations. Gene expression patterns in the c-Kit+ cells were evaluated for increased DNA repair gene expression. Gene expression analyses were performed on sorted CD117+ and CD117− cells from whole bone marrow from the β-globin/GFP mice using Illumina arrays.

TABLE 3
Selected genes that were up-regulated in CD117+ enriched
cells as compared to CD117− cells with increased expression
of transcripts expected to be associated with CD117 including
c-Kit, VEGF (vascular endothelial growth factor), Sca1 (stem
cell antigen-1), and Erdr1 (erythroid differentiation regulator 1).

| | | | Fold Change | |
|---|---|---|---|---|
| | CD117 | CD117 | CD117 negative/ | |
| Gene | Negative | Positive | CD117 positive | P value |
| c-Kit | 593.98 | 2368.32 | −3.98715 | 0.0051 |
| VEGF | 344.34 | 1109.97 | −3.22341 | 0.0084 |
| Sca1 | 208.24 | 490.86 | −2.35711 | 0.0126 |
| Erdr1 | 1011.81 | 2760.26 | 2.72805 | 0.0319 |

Numerous genes involved in DNA repair, including BRCA1, BRCA2, Rad51, ERCC2, XRCC2, XRCC3, showed higher levels of expression in CD117+ cells. Two key HDR genes expected to play a role in PNA-induced recombination, BRCA2 and Rad51, were among the upregulated genes detected by the array. Increased expression of these genes was confirmed in CD117+ cells at the mRNA level by quantitative RT-PCR (FIGS. 2E and 2F) and at the protein level by western blot.

Based on these findings, activation of the c-Kit pathway by SCF treatment to further increase DNA repair gene expression was examined. Gene expression profiling on SCF-treated CD117+ cells versus untreated CD117+ cells showed additional up-regulation of numerous DNA repair genes (FIG. 2G), again including Rad51 and BRCA2.

### Example 6: The c-Kit Pathway Induces Functionally Elevated DNA Repair

### Materials and Methods

### Reporter Gene Assay for Homology-Dependent Repair

An inactivating I-Scel site was cloned 56 amino acids into the firefly luciferase open reading frame under the control of a CMV promoter. The reporter construct also contains a promoterless luciferase gene used as a template for homologous recombination. A double-strand break in the luciferase reporter is created by in vitro digestion with the I-Sce I restriction enzyme (NEB #R0694L). Plasmid DNA was digested with I-Sce 1 for 1 hour at 37° C. at a ratio of 10 units enzyme to 1 μg DNA and then the enzyme was inactivated at 65° C. for 20 minutes. The linearization of the plasmid was confirmed for each digestion via gel electrophoresis and the linear plasmid was purified using the Qiagen Qiaquick spin columns. After separation CD117+ and CD117− cells from bone marrow of β-globin/GFP transgenic mice, cells were transfected using the Lonza 2b Nucleofector Device. 5×10⁵ cells were transfected with 1 μg of either the luciferase reporter vector or a positive control firefly luciferase expression vector, along with 50 ng of a Renilla luciferase expression plasmid as a transfection efficiency control. All transfections were performed in triplicate. After transfection the cells were plated at a density of 5×10⁵ cells/ml in 12-well plates. After 24 hours incubation post transfection, luciferase activity was measured using the Promega Dual Luciferase Assay Kit. In each sample firefly luciferase activity was normalized to the Renilla luciferase transfection control. Reporter reactivation was calculated as a ratio of normalized firefly luciferase activity in the cells transfected with the reporter plasmid to the positive control.

### Results

To test whether the above increases in DNA repair gene expression could be correlated with functional differences in DNA repair, a luciferase-based assay was used to quantify repair of DNA double-strand breaks (DSBs) by HDR. In this assay, repair of a DSB in a reporter plasmid via intramolecular homologous recombination creates (“reactivates”) a functional luciferase gene (FIG. 2H), and so the assay provides a measure of HDR capacity (FIG. 2J). The results show increased luciferase reactivation in CD117+ compared to CD117− cells (FIG. 2H). The repair activity in the CD117+ cells was diminished by treatment with the kinase inhibitors MEK162, BKM120 and dasatnib (FIG. 2H); conversely, it was further boosted by SCF treatment (FIG. 2I). These results indicate that a functional c-Kit signaling pathway mediates increased HDR.

### Example 7: In Vivo Gene Editing by Intravenous Injections of PNA/DNA NPs is Enhanced by SCF Treatment

### Materials and Methods

### Mouse Models and In Vivo Treatments

All animal use was in accordance with the guidelines of the Animal Care and Use Committee of Yale University and conformed to the recommendations in the Guide for the Care and Use of Laboratory Animals (Institute of Laboratory Animal Resources, National Research Council, National Academy of Sciences, 1996).

The β-globin/GFP transgenic mice were obtained from Ryszard Kole, University of North Carolina (Sazani, et al., Nat. Biotechnol., 20:1228-1233 (2002)). For treatment of the mice, where indicated SCF (15.6 ug per mouse, Recombinant Mouse SCF, carrier-free, R&D catalog #455-mc-050/CF) was injected intraperitoneally 3 hrs prior to treatment with 4 mg of NPs in 150 μl PBS delivered via retro-orbital intravenous injection. In some cases, mice were sacrificed 48 hrs after the NP injections and bone marrow and spleen cells were harvested for further analysis. The bone marrow and spleen cells (500,000 each) were co-labelled with APC conjugated antibodies as described above and flow cytometry was performed as above. For deep sequencing analyses, CD117+ cells were isolated based on magnetic separation methods according to BD Bioscience protocol (BDImagTm Hematopoietic Progenitor Stem Cell Enrichment Set—DM), and genomic DNA from three mice was pooled followed by sequence analysis as described (McNeer, et al., Gene Therapy, 20:658-669 (2013)).

The IVS2-654 β-thalassemic mice were also obtained from Ryszard Kole, University of North Carolina (Svasti, et al., Proc Natl Acad Sci USA, 106:1205-1210 (2009)). For treatment of the mice, where indicated SCF (15.6 ug per mouse, Recombinant Mouse SCF, carrier-free, R&D catalog #455-mc-050/CF) was injected intraperitoneally 3 hrs prior to treatment with 4 mg of NPs in 150 μl PBS delivered via retro-orbital intravenous injection. Each mouse received 4 treatments given at 48 hr intervals. Mice were anesthetized with isoflurane followed by retro-orbital bleeding (~100 L) using ethylenediaminetetraacetic acid-treated glass capillary tubes. The blood was evacuated into tubes with 5 μL of 0.5 M EDTA acid in heparinized coated tubes. Complete blood counts were performed using a Hemavet 950FS (Drew Scientific, Oxford, CT) according to the manufacturer's protocol. Slides containing blood smears were stained with Wright and Giemsa stain for microscopy. Methylene blue staining was used for reticulocyte counts. Spleen images and weights were taken after selected mice were sacrificed on day 36 after the last treatment. Harvested spleens were fixed in 10% neutral buffered formalin and processed by Yale Pathology Tissue Services for H&E, CD61 and E cadherin staining.

For assigning animals into treatment groups as listed above, littermate animals were genotyped, and then the pups carrying the required genotypes (either β-globin/GFP transgenic mice or IVS2-654 β-thalassemic mice) were randomized into the several treatment groups in cohorts of 3 to 6, as indicated. The investigators were not blinded as to treatment groups.

### Genomic DNA Extraction and Deep Sequence Analysis

Genomic DNA from mouse cells treated ex vivo or in vivo, as indicated, was harvested using the Wizard Genomic Purification Kit (Promega), and then electrophoresed in a 1% low melting point agarose gel in TAE, to separate genomic DNA from possible residual PNA and/or DNA oligonucleotide. The high-molecular weight species, representing genomic DNA, was cut from the agarose gel and extracted using the Wizard SV Gel and PCR Clean-Up System (Promega) according to manufacturer's instructions. Once genomic DNA was isolated from treated cells or mouse tissue, PCR reactions were performed with high fidelity TAQ polymerase. Each PCR tube consisted of 28.2 μL dH2O, 5 μL 10×HiFi Buffer, 3 μL 50 mM MgCl¬2, 1 μL DNTP, 1 μL each of forward and reverse primer, 0.8 μL High Fidelity Platinum Taq Polymerase (Invitrogen, Carlsbad CA) and 10 μL DNA template. PCR products were prepared by end-repair and adapter ligation according to Illumina protocols (San Diego, CA), and samples sequenced by the Illumina HiSeq with 75 paired-end reads at the Yale Center for Genome Analysis. Samples were analyzed as previously described (McNeer, et al., Gene Therapy, 20:658-669 (2013)). Primers for deep sequencing were designed using Primer3 data base.

### Results

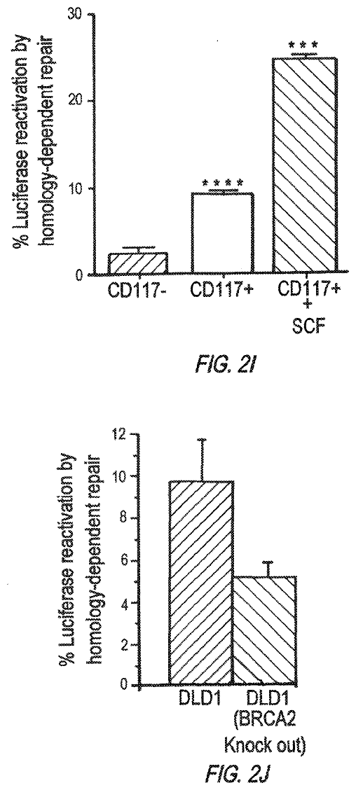

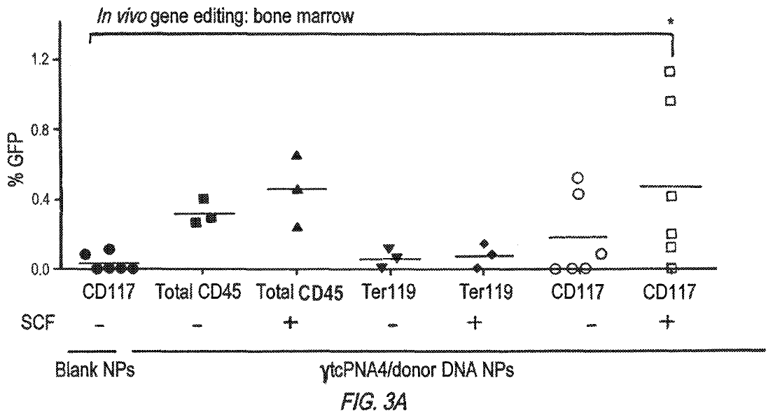

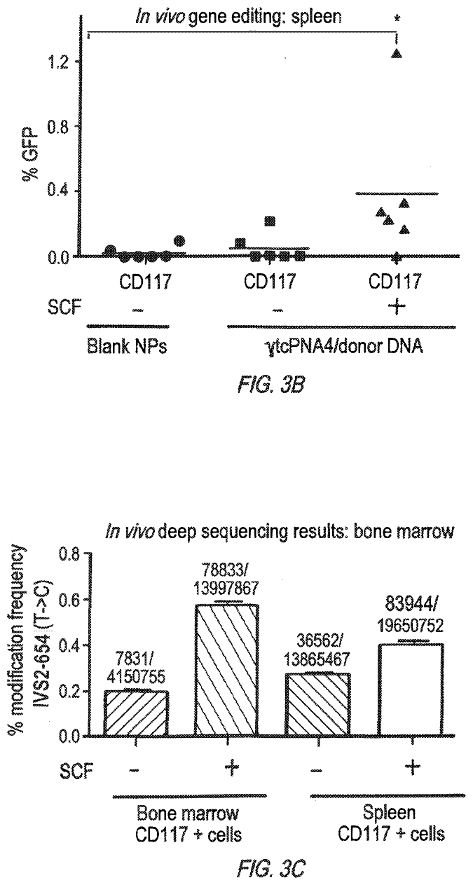

The potential for in vivo gene editing in the β-globin/GFP transgenic mice was explored by intravenous injection of NPs containing γtcPNA4 and donor DNA. The ability of SCF treatment to enhance gene editing in vivo was also tested. Mice were treated with a single intravenous dose of 4 mg NPs in 150 μl PBS, and 2 days later the mice were sacrificed for analysis of gene editing in cells from the bone marrow and spleen. Some mice also received murine SCF (15.6 μg) given by intraperitoneal injection 3 hr prior to the NP injection, as indicated. In vivo gene editing was scored by GFP expression in marker-sorted cell populations from bone marrow and spleen (FIGS. 3A and B). The highest levels of gene editing were seen in CD117+ cells from bone marrow and spleen of the SCF-treated mice, with frequencies in the range of 1% in several mice, and average frequencies in the 0.4% to 0.5% range.

These results were confirmed by performing deep sequencing analysis on genomic DNA from CD117+ cells isolated from bone marrow and spleen of treated mice (FIG. 3C), which revealed gene editing frequencies in the range of 0.2% in the bone marrow of mice treated with NPs alone and 0.6% in mice receiving SCF along with the NPs, consistent with the frequencies of gene correction quantified by GFP expression. Deep-sequencing was also used to assess off-target effects in the bone marrow cells of the mice that were treated with SCF and γtcPNA4 and donor DNA NPs (Table 4). By BLAST analysis, seven off-target sites with partial homology to the target site of γtcPNA4 in β-globin intron 2 were identified. Mutation frequencies at these sites were quantified via deep sequencing. Extremely low frequencies of off-target effects were found in the γtcPNA4/donor DNA treated mice, with six sites showing no detectable sequence changes out of millions of reads and two sites showing modification frequencies of only 0.0074% and 0.00018% compared to 0.56% at the targeted β-globin site. (Table 4). The overall off-target modification frequency at all seven sites combined was 0.00034%, 1,647-fold lower than the frequency of the targeted gene editing.

TABLE 4
Off-target effects in bone marrow cells following intravenous
treatment of β-globin/GFP mice with γtcPNA4/donor DNA NPs.

| | | Size of | | | |
|---|---|---|---|---|---|
| | Sequences of partial | region | Alleles | Number | |
| Gene locus | homology (5′ to 3′) | sequenced | sequenced | modified | Frequency % |
| β-globin | TGCCCTGAAAGAAAGA | 128 | 1399786 | 78833 | 0.56 |
| | GA (SEQ ID NO: 16) | | | | |
| Vascular cell | AGCCCTGAAAGAAAG | 111 | 480013 | 0 | 0 |
| adhesion | AGA (SEQ ID NO: 196) | | | | |
| protein | | | | | |
| precursor 1 | | | | | |
| Polypyrimidine | GAACCTGAAAGAAAG | 101 | 349723 | 26 | 0.0074 |
| tract binding | AGA (SEQ ID NO: 197) | | | | |
| protein | | | | | |
| Protocadherin | CACCCTGAAAGAAAGA | 115 | 73245 | 0 | 0 |
| fat 4 precursor | AA (SEQ ID NO: 198) | | | | |
| Olfactory | AAGCCTGAAAGAAAG | 172 | 1092990 | 2 | 0.00018 |
| receptor | AGT (SEQ ID NO: 199) | | | | |
| 266 | | | | | |
| Syntaxin | AGAAATGAAAGAAAG | 150 | 2478636 | 0 | 0 |
| binding | AGA (SEQ ID NO: 200) | | | | |
| protein | | | | | |
| Muscleblind | GGTGGTGAAAGAAAG | 165 | 2331971 | 0 | 0 |
| like protein | AGA (SEQ ID NO: 201) | | | | |
| Ceruloplasmin | AGGACTGAAAGAAAG | 154 | 1390439 | 0 | 0 |
| isoform | AGT (SEQ ID NO: 202) | | | | |
| Total off-target | | | 8197017 | 28 | 0.00034 |

The top seven gene loci with partial homology to the 18 bp γtcPNA4 target site in β-globin intron 2 were identified, with the sequences as indicated. β-globin/GFP mice were treated with SCF followed by intravenous infusion with NPs containing γtcPNA4/donor DNA, and genomic DNA from c-Kit+ bone marrow cells was subject to deep sequencing analysis at these loci. The size of the region sequenced around each site is listed, along with the number of alleles sequenced and the number of alleles with modified sequences.

### Example 8: SCF and PNA NP Treatment can Correct a Genomic Mutation in a Mouse β-Thalassemia Disease Model

To test the extent to which combined SCF and PNA NP treatment in vivo could correct a human β-thalassemia mutation in a mouse disease model, a transgenic mouse line was utilized in which the two (cis) murine adult beta globin genes were replaced with a single copy of the human β-globin gene with the thalassemia-associated IVS2-654 mutation (Svasti, et al., Proc Natl AcadSci USA, 106:1205-1210 (2009)). Homozygous mice do not survive, and heterozygotes have a moderate form of β-thalassemia, with marked hemolytic anemia, microcytosis, and increased MCHC and red cell distribution width reflecting reduced amounts of mouse β-globin and no human β-globin (Lewis, et al., Blood, 91:2152-2156 (1998); Svasti, et al., Proc Natl Acad Sci USA, 106:1205-1210 (2009)). Blood smears from these mice show erythrocyte morphologies consistent with β-thalassemia.

### Materials and Methods

Treatment groups for this experiment included (1) blank NPs; (2) SCF treatment alone (no NPs); (3) SCF plus γtcPNA4/donor DNA NPs; and (4) SCF plus γtcPNA4-Scr/donor DNA. SCF injections were given i.p., and NPs were given i.v. via retro-orbital injection. Each treatment group consisted of six mice, and each mouse received four treatments at two-day intervals. Blood smears examined at day 0 (before treatment) and at day 36 after the last treatment showed marked improvement in RBC morphology on day 36 in the γtcPNA4/donor DNA treated mice but not in the mice treated with either blank NPs, SCF alone, or SCF plus γtcPNA4-Scr/donor DNA. Compared to wild-type, the untreated group (and corresponding control animals) exhibit extreme poikilocytosis which is typical of β-thalassemia, as well as the presence of numerous target cells, cabot rings, anisochromasia, and ovalocytosis, changes characteristic of β-thalassemia. Treatment with γtcPNA4/donor DNA and SCF ameliorates the poikilocytosis and yields a reduction in anisocytosis, ovalocytosis, and target cells indicative of reduced alpha-globin precipitation in the RBCs.

### Results

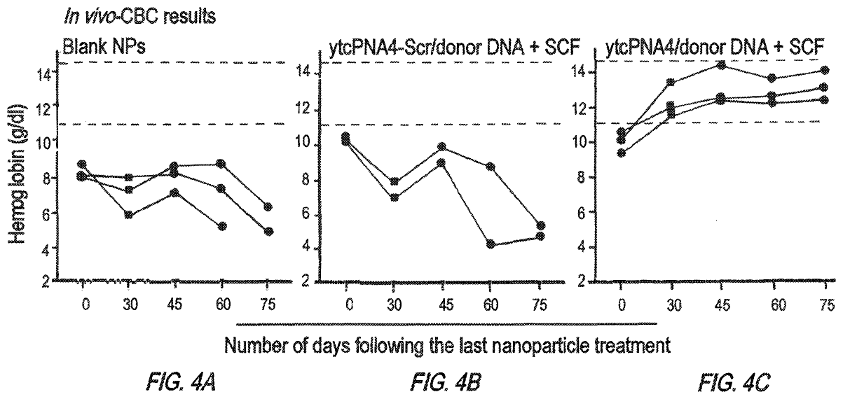

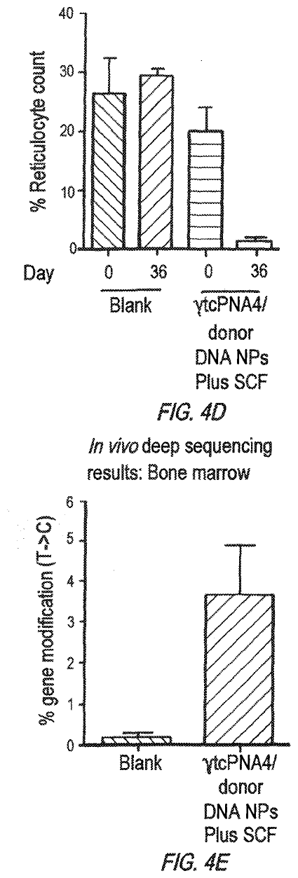

CBC analyses performed on blood samples taken at 30, 45, 60, and 75 days post-treatment from mice in each group showed persistent correction of the anemia in the mice treated with SCF plus the γtcPNA4/donor DNA NPs (FIG. 4A-4C), with elevation of the blood hemoglobin levels into the normal range. Only the SCF plus γtcPNA4/donor DNA-treated mice achieved and maintained hemoglobin levels within the normal range during the duration of the experiment, reflecting the increased hemoglobin stability conferred by the gene editing.

The anemia was not improved in any of the controls. Reticulocyte counts were observed in mice treated with SCF plus the γtcPNA4/donor DNA NPs but not in the mice treated with blank NPs (FIG. 4D). Deep sequencing analyses were performed on genomic DNA extracted from bone marrow cells of three mice from each group that were sacrificed on day 36 post-treatment. Correction of the targeted mutation was seen at a frequency of almost 4% in the γtcPNA4/donor DNA treated group (FIG. 4E), whereas no correction was seen in the mice treated with blank NPs. In addition, in keeping with the correction of the anemia and suppression of the reticulocytosis, the γtcPNA4/donor DNA treated mice also showed reduced splenomegaly at 36 days post-treatment.

Consistent with the reduced splenomegaly, histologic examination of the spleens of mice sacrificed on day 36 showed substantially improved splenic architecture specifically in the γtcPNA4/donor DNA treated mice. The regular splenic histologic pattern of white pulp (lymphoid follicles) surrounded by rims of red pulp as seen in the wild-type spleen is disrupted in the β-thalassemic animals (blank NPs, SCF alone, SCF plus scrambled γtcPNA4-Scr/donor DNA NPs) due to extramedullary hematopoiesis, which results in an expansion in the red pulp (causing the splenomegaly) and disruption of the white pulp. The CD61 and Ecad immunohistochemical stains highlight the increased cellularity characteristic of extramedullary hematopoiesis and demonstrate that the expanded red pulp in the 3-thalassemic animals includes elevated numbers of megakaryocytes and erythroid precursors, respectively. This increased cellularity is substantially ameliorated in the γtcPNA4/donor DNA treated mice.

Deep-sequencing was also used to assess off-target effects in the bone marrow of the in vivo treated thalassemic mice. As above, seven off-target sites with partial homology to the binding site of γtcPNA4 in the β-globin gene were analyzed. Only extremely low frequencies of off-target effects were found in the γtcPNA4/donor DNA-treated thalassemic mice (Table 5), similar to the results in the β-globin/GFP transgenic mice (Table 4). The overall off-target modification frequency in this case was 0.0032%, 1,218-fold lower than the frequency of β-globin gene editing.

TABLE 5
Off-target effects in bone marrow cells following intravenous
treatment of β-thalassemic mice with SCF and γtcPNA4/donor DNA NPs.

| | | Size of | | | |
|---|---|---|---|---|---|
| | Sequences of partial | region | Alleles | Number | |
| Gene locus | homology (5′ to 3′) | sequenced | sequenced | modified | Frequency % |
| β-globin | TGCCCTGAAAGAAAGAGA | 128 | 8615313 | 337192 | 3.9 |
| | (SEQ ID NO: 16) | | | | |
| Vascular cell | AGCCCTGAAAGAAAGAG | 111 | 482051 | 0 | 0 |
| adhesion | A (SEQ ID NO: 196) | | | | |
| protein | | | | | |
| precursor 1 | | | | | |
| Polypyrimidine | GAACCTGAAAGAAAGAG | 101 | 355567 | 2 | .00056 |
| tract binding | A (SEQ ID NO: 197) | | | | |
| protein | | | | | |
| Protocadherin | CACCCTGAAAGAAAGAAA | 115 | 123158 | 0 | 0 |
| fat 4 precursor | (SEQ ID NO: 198) | | | | |
| Olfactory | AAGCCTGAAAGAAAGAG | 172 | 1099880 | 262 | 0.0231 |
| receptor | T (SEQ ID NO: 199) | | | | |
| 266 | | | | | |
| Syntaxin | AGAAATGAAAGAAAGAG | 150 | 2493024 | 0 | 0 |
| binding protein | A (SEQ ID NO: 200) | | | | |
| Muscleblind like | GGTGGTGAAAGAAAGAG | 165 | 2336715 | 0 | 0 |
| protein | A (SEQ ID NO: 201) | | | | |
| Ceruloplasmin | AGGACTGAAAGAAAGAG | 154 | 1397271 | 0 | 0 |
| isoform | T (SEQ ID NO: 202) | | | | |
| Total off-target | | | 8287666 | 268 | .0032 |

The top seven gene loci with partial homology to the 18 bp γtc13NA4 target site in β-globin intron 2 were identified, with the sequences as indicated. Thalassemic mice were treated with SCF followed by intravenous infusion with NPs containing γtcPNA4/donor DNA, and genomic DNA from c-Kit+ bone marrow cells was subject to deep sequencing analysis at these loci. The size of the region sequenced around each site is listed, along with the number of alleles sequenced and the number of alleles with modified sequences.

The results above demonstrate that chemically modified γPNAs and donor DNAs delivered intravenously via polymer NPs, and given in combination with SCF treatment, can mediate gene editing in vivo at a level sufficient to ameliorate the disease phenotype in the thalassemic mice. Sustained reversal of the anemia, with normalization of serum hemoglobin concentrations and suppression of the reticulocytosis were induced. A morphologic improvement in RBC cytology, indicative of improved RBC stability, along with reduced extramedullary hematopoiesis and reduction in splenomegaly were observed. This constellation of findings indicates that the therapeutic approach has the potential to deliver a substantial clinical response that would relieve the morbidity and mortality associated with (3-thalassemia.

There are at least two important advances for gene editing in this study. One advance is the incorporation of next generation PNA chemistry by substitution within the polyamide backbone at the gamma position to consistently yield increases in gene editing frequencies compared to standard PNAs. This increased efficacy correlates with the enhanced DNA binding properties of γPNAs, which take on a pre-organized helical conformation enforced by the miniPEG γ substitution.

Another advance is the finding that the SCF/c-Kit pathway promotes increased gene editing by triplex-forming PNAs and donor DNAs. Upon ex vivo treatment of bone marrow cells with γPNAs, the gene editing frequency in c-Kit+ cells was as high as 8%. The combination of SCF treatment with the γPNAs yielded even higher frequencies in the c-Kit+ cells, with just over 15% in a single treatment. In vivo, treatment of transgenic mice carrying a β-globin/GFP reporter transgene by i.p. injection of SCF followed by intravenous administration of NPs containing γPNAs and donor DNAs yielded gene editing in CD117+ cells in the bone marrow and spleen at frequencies up to 1% in a single treatment. Prompted by these results in reporter mice, gene editing was tested in the thalassemic mouse model via simple intravenous injection of the optimized combination of SCF and γPNA/donor DNA NPs given four times at two-day intervals. This regimen yielded gene editing at a frequency of almost 4% in total bone marrow cells and produced sustained amelioration of the disease phenotype, achieved in a minimally invasive manner without the need for stem cell harvest or transplantation.

In a series of ex vivo and in vivo assays for hematopoietic colony formation, for induction of inflammatory cytokines, for generation of strand breaks, and for off-target mutagenesis by deep sequencing, there was essentially no measurable cellular toxicity and very low off-target genome effects from the γPNA-containing NPs, providing a possible safety advantage relative to other gene editing approaches (Cradick, et al., Nucleic Acids Res., 41:9584-9592 (2013)).

CD117 is the product of the c-Kit gene and is a receptor tyrosine kinase that mediates downstream signalling to multiple cellular pathways. The results discussed above indicate that activation of this pathway promotes gene editing, rather than CD117 simply being a marker for the phenotype. Inhibition of the c-Kit kinase with dasatinib reduces the frequency by almost 4-fold, whereas treatment with SCF almost doubles the frequency. Mechanistically, CD117+ bone marrow cells, in comparison to CD117− cells, have elevated levels of expression of numerous DNA repair genes, including factors in the HDR pathway that prior work has shown is required for triplex-induced gene editing (Vasquez, et al., Science, 290:530-533 (2000); Rogers, et al., Proc. Natl. Acad. Sci. USA, 99:16695-16700 (2002); Datta, et al., J Biol Chem, 276:18018-18023 (2001); Vasquez, et al., Proc Natl Acad Sci USA, 99:5848-5853 (2002)). When CD117+ cells are treated with SCF, expression of these DNA repair genes is increased even more, correlating with a further increase in gene editing.

In addition, the results show that the elevated expression of DNA repair genes in CD117+ cells is associated with functionally increased HDR activity using an assay for recombination between reporter gene constructs. Treatment of the CD117+ cells with SCF produced a further 2-fold increase in HDR, whereas dasatinib and the other inhibitors yielded reductions in HDR activity. These results show the functional importance of the c-Kit pathway in promoting HDR and provide further mechanistic insight into gene editing pathways.

The 4% frequency of bone marrow gene editing achieved in the thalassemic mice was sufficient to achieve a clear improvement in phenotype, with blood hemoglobin levels rising into the normal range, suppression of the reticulocytosis, and reduction in the splenomegaly that is otherwise associated with extramedullary hematopoiesis. The observation that gene correction at a frequency of 4% could confer a phenotypic impact is consistent with transplantation studies in thalassemic mice and in patients in which mixed chimerism at one ratio of wild-type donor to thalassemic recipient cells in the marrow has produced much higher proportions of donor RBCs in the periphery (Andreani, et al., Bone Marrow Transplant, 7(Suppl 2):75 (1991); Felfly, et al., Mol Ther, 15:1701-1709 (2007)). This effect has been attributed to increased survival and enrichment of genetically corrected erythroblasts during erythropoiesis, decreased ineffective erythropoiesis, and increased survival in the circulation of corrected erythrocytes relative to thalassemic RBCs (Miccio, et al., Proc Natl Acad Sci USA, 105:10547-10552 (2008)).

Overall, these results show the feasibility of NP-mediated delivery of γPNAs and donor DNAs as a therapeutic strategy to achieve in vivo gene editing for treatment of human genetic disorders. The results demonstrate effective NP-mediated gene editing in bone marrow. Other recent work has shown that NP delivery to lung airway epithelia is also possible as a means to achieve correction of the CFTR gene mutation associated with cystic fibrosis (Fields, et al., Adv Healthc Mater (2014); McNeer, et al., Nature Communications in press (2015)).

The finding that SCF stimulates gene editing identifies SCF as a pharmacologic means to boost gene editing, a strategy that should be applicable not just to PNA-mediated gene editing as well as other methods, such as CRISPR/Cas9, SFHR, or ZFNs. Even though the γPNAs show consistently improved gene editing potency, the level of off-target effects in the genome remains extremely low. This is in keeping with the lack of any intrinsic nuclease activity in the PNAs (in contrast to ZFNs or CRISPR/Cas9), and reflects the mechanism of triplex-induced gene editing, which acts by creating an altered helix at the target-binding site that engages endogenous high fidelity DNA repair pathways. The SCF/c-Kit pathway also stimulates these same pathways, providing for enhanced gene editing without increasing off-target risk or cellular toxicity.

### Example 9: Repair Proteins Modulate Triplex-Forming PNA Mediated Gene Editing

### Materials and Methods

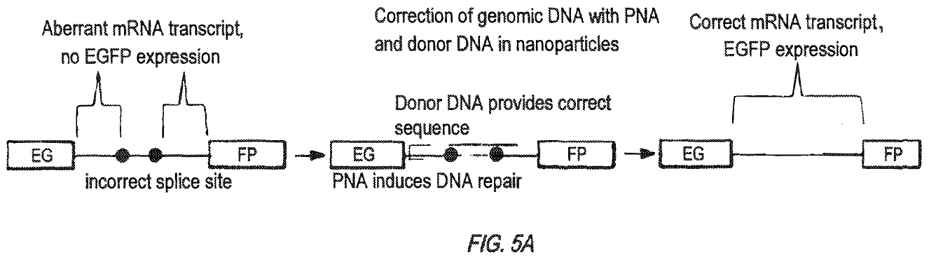

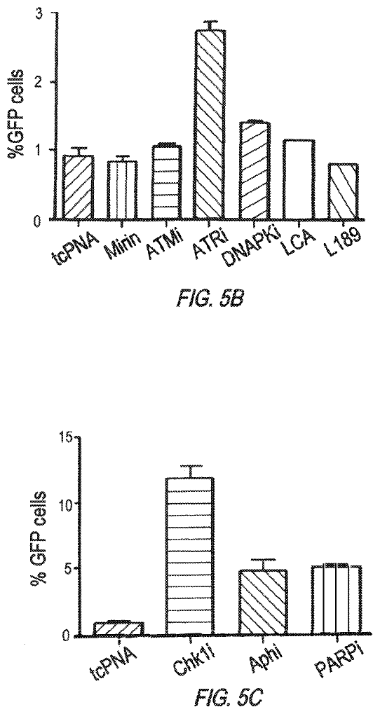

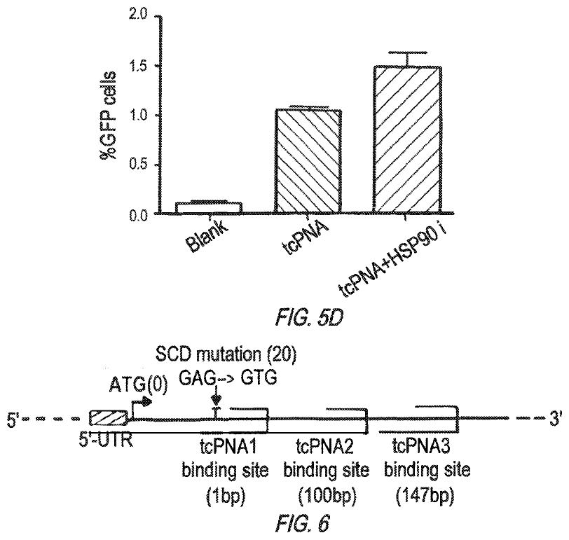

Skin fibroblasts were isolated from the β-globin/GFP mice (intron 2 of human β-globin inserted with in the GFP coding regions) and grown in culture in DMEM medium plus 10% FCS. The intron contains the IVS2-654 (C->T) mutation. The gene correction assay is illustrated in FIG. 5A.

The fibroblasts were treated ex vivo with nanoparticles containing tcPNA1+Donor DNA and 72 hours later flow cytometry analysis was performed to quantify the % gene correction based on the frequency of GFP positive cells. In some cases, DNA repair inhibitors or other small molecule inhibitors were given 48 hours before the nanoparticle treatment.

TABLE 6
ATR pathway inhibitors

| | | Working |
|---|---|---|
| Drug | Inhibits | Concentration |
| MIRIN | Mre11 | 20 μM |
| KU55933 | ATM | 20 μM |
| VE-821 | ATR | 10 μM |
| NU7441 | DNAPKcs | 20 μM |
| LCA | Polymerase β | 50 μM |
| L189 | DNA ligase I III IV | 50 μM |

TABLE 7
CHK1, DNA polymerase alpha, and polyADP
ribose polymerase inhibitors

| | | Working | |
|---|---|---|---|
| Drug | Inhibits | Concentration | |
| Aphidicolin | Polymerase α | 1 | μg/ml |
| SB218075 | Chk1 | 1 | μM |
| AZD | PARP | 20 | μM |

### Results

Inhibition of ATR boosts gene editing in the GFP/beta globin gene correction assay in mouse fibroblasts. The results are presented in FIG. 5B.

Inhibition of CHK1 substantially boosts gene editing in GFP/beta globin gene correction assay. Inhibition of DNA polymerase alpha (by aphidicolin) or of polyADP ribose polymerase by AZD-2281 (olaparib) also boosts gene editing. The results are presented in FIG. 5C.

Inhibition of heat shock protein 90 (HSP90) by STA-9090/Ganetespib enhances gene editing in the GFP/beta globin gene correction assay. The results are presented in FIG. 5D.

### Example 10: Partial γ Substitution in the Hoogsteen Domain Increases Gene Correction Efficiency

### Results

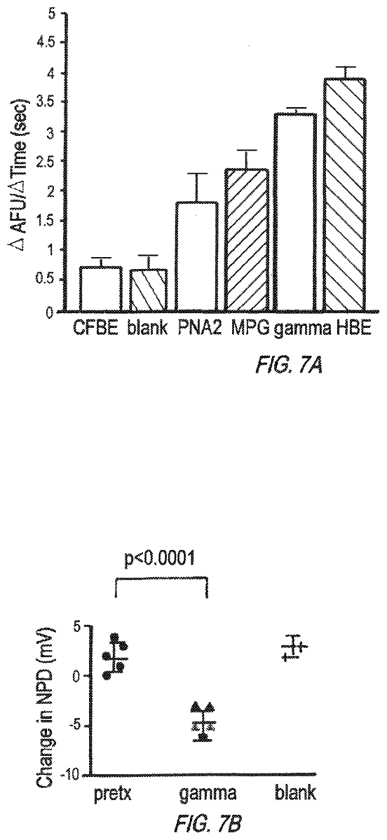

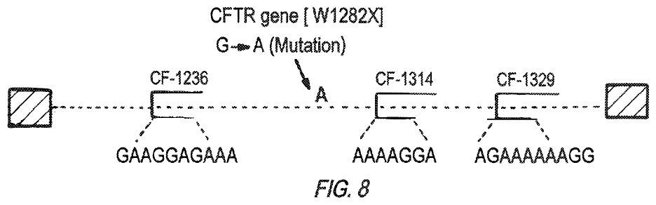

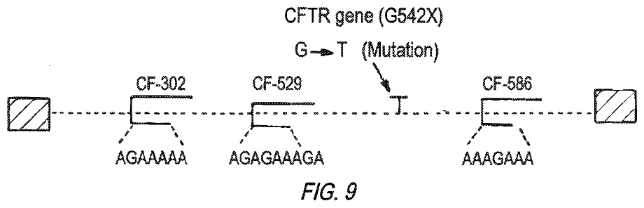

A substantial increase in gene editing in F508del CFTR using γtcPNAs with just partial γ substitution and only in the Hoogsteen domain of CF PNA2. As shown in FIGS. 7A and 7B, with only 4 γ residues in the Hoogsteen domain, a more than 50% increase in activity for CFTR gene correction was achieved in CFBE cells (via NPs containing γtcPNAs) as judged by the MQAE assay (FIG. 7A). A substantial increase in activity with γtcPNA containing NPs was also achieved in vivo in CF mice following intranasal delivery, as determined by NPD measurements (FIG. 7B).

### Example 11: Nanoparticle Delivered tcPNA and Donor Oligonucleotide Correct a Sickle Cell Mutation In Vivo

### Materials and Methods

### Mouse Models for Sickle Cells Disease

In sickle cell disease (SCD), the mutation (GAG->GTG) at codon 6 results in glutamic acid changed to valine. For correction of this SCD mutation site in vivo, in vivo studies were performed in two mouse models:
(1) sickle cell gene knock in murine model also known as the Berkeley mouse model introduced by Pászty C, Brion C M, Manci E, Witkowska H E, Stevens M E, Mohandas N, Rubin E M., “Transgenic knockout mice with exclusively human sickle hemoglobin and sickle cell disease.” Science. 1997 Oct. 31; 278(5339):876-8. PMID: 9346488 and
(2) the Townes mouse model developed by Ryan™, Ciavatta D J, Townes T M, “Knockout-transgenic mouse model of sickle cell disease.” Science. 1997 Oct. 31; 278(5339):873-6. PMID: 9346487.

Both of these mouse models express exclusively human sickle hemoglobin (HbS). They were produced by generating transgenic mice expressing human α-, γ-, and β^{s}-globin that were then bred with knockout mice that had deletions of the murine α- and β-globin genes. Thus the resulting progeny no longer express mouse α- and β-globin. Instead, they express exclusively human α- and β^{s}-globin. Hence, the mice express human sickle hemoglobin and possess many of the major hematologic and histopathologic features of individuals with SCD.

### Nanoparticles

tcPNAs and donor DNAs, at a molar ratio of 2:1, were incorporated into PLGA NPs. The NP formulations were evaluated by scanning electron microscopy (SEM) and dynamic light scattering (DLS).

### Treatment Protocol

Three each (i.e., n=3) of Berkley and Townes mice were treated with (1) Blank PLGA nanoparticles, (2) 4 treatments of sc-tcPNA1/donor DNA in PLGA nanoparticles, (3) 4 treatments of sc-tcPNA2/donor DNA in PLGA nanoparticles, (4) 4 treatments of sc-tcPNA3/donor DNA in PLGA nanoparticles. Mice were injected intravenously with 2 mg of NPs containing the PNAs and donor DNAs every two days for a total of 4 injections. After the last treatment, bone marrow and spleen were collected for histology, deep sequencing, and restriction enzyme digest.

### Results

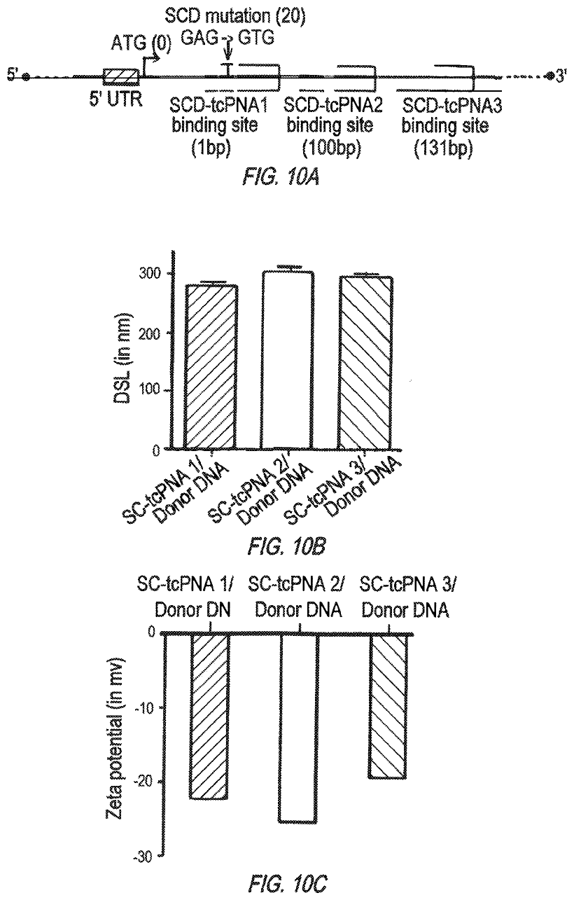

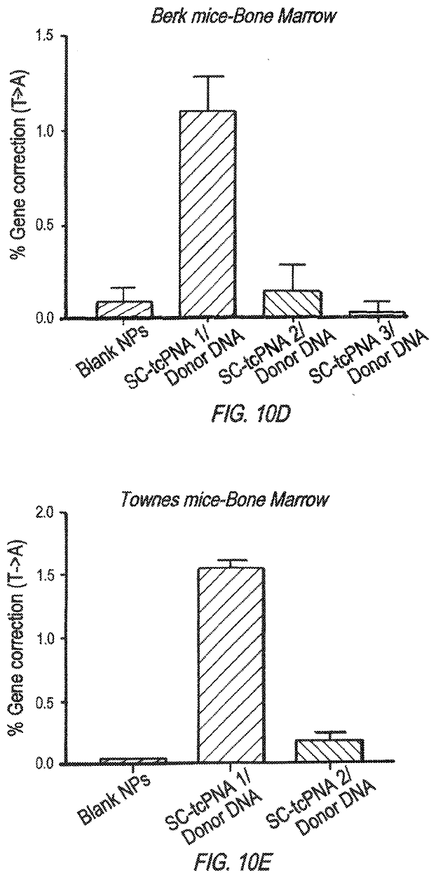

Three polypurine sites in the β-globin gene in the vicinity of the SCD codon. Triplex formation can catalyze recombination at sites up to several hundred base pairs away. A series of tcPNAs were designed to bind to selected polypurine stretches in the β-globin gene in the vicinity of the SCD mutation and synthesized (FIG. 10A). A sense donor DNA (a single-stranded 60-mer matching nucleotides in β-globin gene and end-protected from degradation by 3 terminal phosphorothioate internucleoside linkages was also designed.

Gel mobility shift assays demonstrated binding of SCDtcPNA1, SCDtcPNA2, SCDtcPNA3 to 120 bp double-stranded DNA fragments containing β-globin sequences. Each 120 bp dsDNA contained the binding site for the respective tcPNAs. The binding assays revealed that all synthesized SCD tcPNAs bind specifically to double-stranded genomic DNA under physiological conditions.

Poly (lactic-co-glycolic acid) (PLGA) NPs can effectively deliver PNA/donor DNA combinations into primary human and mouse hematopoietic cells with essentially no toxicity. Here, tcPNAs and donor DNAs, at a molar ratio of 2:1, were incorporated into PLGA NPs. The NP formulations were evaluated by scanning electron microscopy (SEM) and dynamic light scattering (DLS). All the NPs exhibited sizes within the expected range and showed uniform charge distribution (FIGS. 10B-10C).

Next, correction of SCD mutation in the two disease mouse models was carried out as described above. Treatment groups included (1) blank NPs; (2) SCD tcPNA1/donor DNA; (3) SCD tcPNA2/donor DNA; and (4) SCD tcPNA3/donor DNA. Mice were injected intravenously with 2 mg of NPs containing the PNAs and DNAs every two days for a total of 4 injections.

Deep sequencing analyses of the human beta globin alleles were performed on genomic DNA taken from total bone marrow cells of mice on day 36 post-treatment. Correction of the SCD mutation was seen at a frequency of almost 1.5% in the SCD tcPNA1/donor DNA treated group in the Townes mice (FIG. 10D) and 1.2% gene correction in the Berkley mice (FIG. 10E), whereas no correction was seen in the mice treated with blank NPs. The results were confirmed using restriction enzyme (Bsu361) digestion which cuts only when the sequence at codon 6 has been edited from the SCD mutation to the wild-type sequence.

Sequences with γPNA substitutions based on the above SCD PNAs can be and have been designed, and include, for example, partial or complete γPNA substitution in the Watson-Crick domain, partial or complete substitutions in the Hoogsteen domain, or a combination thereof.

### Example 12: In Utero Gene Correction Cures β-Thalassemia in Mice

### Materials and Methods

### Oligonucleotides

Mini-PEG γPNA monomers were prepared as previously described (Bahal R et al., Chembiochem 13, 56-60 (2012)). PNA oligomers were synthesized, purified, and characterized as shown in preceding Examples (Bahal R et al., Nat Commun 7, 13304 (2016)).

### PLGA Nanoparticle Synthesis and Characterization

PLGA (50:50 ester-terminated, 0.95-1.2 g/dl, LACTEL absorbable polymers; Birmingham, AL) NPs containing C6 (Sigma; St Louis, MO) or DiD (Thermo Scientific; Rockford, IL) were synthesized using a previously described single-emulsion solvent evaporation technique (McNeer N A et al., Mol Ther 19, 172-180 (2011)). C6 or DiD was added to the polymer solution at a 0.2% wt:wt dye:polymer ratio. PNA/DNA and blank PLGA NPs were synthesized using a previously described double-emulsion solvent evaporation technique modified to encapsulate PNA and DNA oligomers (Ricciardi A S et al., Methods Mol Biol 1176, 89-106 (2014)). All PNA/DNA NP batches were formulated with 2 nmole mg⁻¹ of PNA and 1 nmole mg⁻¹ DNA. Blank NPs were loaded with phosphate-buffered saline. Scanning electron microscopy (SEM) was performed using an XL-30 scanning electron microscope (FEI; Hillsboro, Oregon) as previously described. Dynamic light scattering (DLS) was performed to measure the NPs size (hydrodynamic diameter) and surface charge (zeta potential) using a Malvern Nano-ZS (Malvern Instruments, UK). Nucleic acid release was analyzed as previously described.

### Mouse Models and Genotyping

All animal use was in accordance with the guidelines of the Animal Care and Use Committee of Yale University and conformed to the recommendations in the Guide for the Care and Use of Laboratory Animals (Institute of Laboratory Animal Resources, National Research Council, National Academy of Sciences, 1996). C57BL/6 mice were obtained from Charles River Laboratories (Wilmington, MA). The IVS2-654 β-thalassemia mice were obtained from Ryszard Kole, University of North Carolina (Chapel Hill, NC) (Lewis, et al., Blood, 91:2152-2156 (1998)). Litters from this mouse model were genotyped prior to weaning. Genomic DNA (gDNA) was isolated from tail clippings using the Wizard SV DNA Purification System (Promega; Madison, WI). Genotyping PCR was performed to detect the presence of the human β-globin gene, indicating the mouse has a Hbb^{th-4}/Hbb⁺ genotype, using a species independent forward primer (complementary to both mouse and human β-globin sequences) and two species dependent reverse primers. PCR was performed using high fidelity Platinum TAQ polymerase (Invitrogen; Carlsbad, CA); reaction conditions are as follows: 5.0 μl 10×HiFi buffer, 3.0 μl MgCl₂, 1.0 μl dNTPs, 2.0 μl 10 μM forward primer, 1.0 μl 10 μM human reverse primer, 1.0 μl 10 μM mouse reverse primer, 0.8 μl HiFi Taq, 90-400 ng gDNA and remaining volume to 50 μl with dH2O. Thermocycler conditions were as follows: 94° C. 2 min, [94° C. 30 s, 55° C. 45 s, 68° C. 1 min]×35 cycles, 68° C. 1 min, hold at 4° C. PCR products were run on a 2% agarose gel. The amplicon derived from the mouse reverse primer is 196 bp and the amplicon from the human reverse primer is 508 bp. The presence of bands at both 196 and 508 bp indicates the Hbb^{th-4}/Hbb⁺ genotype. A single band at 196 bp indicates the Hbb⁺/Hbb⁺ genotype. Only heterozygous mice were included in this study.

### In Utero NP Delivery and Imaging

Time dated pregnant mice (8-12 weeks old) between 15 to 18 days post conception were anesthetized with inhaled isoflurane (3% vol/vol for induction, 2% vol/vol for maintenance). The gravid uterus was exposed through a midline laparotomy incision. For the biodistribution studies, lyophilized fluorescent nanoparticles were re-suspended by vortex and water bath sonication in 1×dPBS to a concentration of 9 mg/ml. Intravascular injections were performed at E15.5 and E16.5. A volume of 15 μl of 9 mg/ml NP suspension was drawn up into a glass micropipette (tip diameter ~60 μm) and injected intravascularly via vitelline vein of each fetus using a pneumatic microinjector (Narishige; Japan). Intra-amniotic injections were performed at E15.5, E16.5, E17.5 and E18.5. A volume of 20 μl of 9 mg/ml NP suspension was injected directly into the amniotic cavity of each fetus. Pregnant mice were sacrificed three hours post DiD PLGA NP injection and fluorescence and x-ray imaging was performed on a Carestream In-Vivo MS FX PRO (Bruker; Billerica, MA). Pregnant mice were also sacrificed 3 hours post C6 PLGA NP injection. Fetuses were delivered via cesarean section and washed in PBS. Ex vivo fetal fluorescence stereomicrope imaging was performed on a Leica M80 stereomicroscope (Wetzlar, Germany). Fetuses and maternal organs were fixed overnight in 4% paraformaldehyde (Electron Microscopy Sciences; Hartfield, PA) at 4° C. The tissues were next dehydrated in 20% sucrose and embedded in Optimal Cutting Temperature (OCT) Compound (Torrance, CA). Frozen 15 μm thick fetal and maternal liver sections were mounted on glass slides and stained with Hoescht dye. Confocal imaging of the frozen sections was performed on a Zeiss Axio Observer Z1 microscope (Oberkochen, Germany).

For the safety studies, the fetuses of time dated pregnant C57BL/6 females (8-10 weeks old) were injected intravenously at E15.5 as described above with 15 μl of 9 mg/ml blank NPs or PNA/DNA NPs. Intra-amniotic injections were performed at E16.5; 20 μl of 9 mg/ml blank NPs or PNA/DNA NPs were injected into the amniotic cavity. Untreated pregnant mice were anesthetized and the gravid uterus was exposed as described above. The fetuses were counted, the uterus was returned to the abdomen, and the midline incision was closed. The number of untreated, intravenously and intraamniotically treated pups surviving was counted at the time of weaning, 21 days. The weight of injected and untreated control pups was measured for a period of 10 months. The fetuses of time dated pregnant Hbb^{th-4}/Hbb⁺ mice (mated with Hbb^{th-4}/Hbb⁺ males) were injected intravenously as described above with 15 μl of either 9 mg/ml or 12 mg/ml PNA/DNA NPs, correlating to doses of 300 mg kg⁻¹ or 400 mg kg⁻¹, respectively.

### Cytokine Array Analysis

The fetuses of time dated pregnant C57BL/6 females (8-10 weeks old) were injected intravenously at E15.5 as described above with 15 μl of blank NPs (9 mg/ml), PNA/DNA NPs (9 mg/ml), or 1×dPBS. After 48 hours, fetal plasma samples were collected. Plasma samples from fetuses receiving PBS, blank NPs, or PNA/DNA NPs and untreated fetuses were submitted to the CytoPlex Core Facility at Yale University. The facility performed luminex based cytokine detection and quantification using the Bio-Plex Pro Mouse Cytokine 23-Plex assay available from Bio-Rad (Hercules, CA).

### Peripheral Blood Analysis

Mice were anesthetized using open-drop 30% w/v isoflurane in propylene glycol. A volume of 50-100 μl of blood was collected retro-orbitally using heparinized microhematocrit capillary tubes (Fisher Scientific; Pittsburgh, PA) and evacuated into heparinized coated tubes containing 5 μl 0.5 M EDTA acid. Complete blood counts were performed using a Hemavet 950FS (Drew Scientific; Oxford, CT) according to the manufacturer's protocol. A volume of 1-3 μl of fresh blood was smeared onto glass slides and stained with Wright-Giemsa stain (Sigma-Aldrich; St. Louis, MO) for 20 s. Slides were washed in 1×dPBS for 10 minutes and air-dried. An additional 10 μl of blood was incubated with 3 μl new methlylene blue reticulocyte stain (Sigma-Aldrich; St. Louis, MO) for 10 min after which blood smears were prepared. A cover slip was mounted on air-dried smears with Cytoseal 60 (Thermo Scientific; Rockford, IL). All blood smears were imaged on an Olympus FSX100 microscope. Two individuals independently counted the number of reticulocytes present in 500 cells. The relative reticulocyte count was calculated as the number reticulocytes in 1000 RBCs divided by ten.

### Histology

Spleen images were taken and weights were recorded for some Hbb^{th-4}/Hbb⁺ mice 15-30 weeks post PNA/DNA NP delivery, age matched untreated Hbb^{th-4}/Hbb⁺ mice, and wild-type mice. Harvested spleens were fixed in 10% neutral buffered formalin and processed by Yale Pathology Tissue Services for H&E staining as well as E-cadherin and CD-61 immunohistochemistry. Spleen sections were imaged on an Olympus FSX100 microscope.

### Fetal Bone Marrow Harvest and Cell Sorting

The bone marrow of untreated and PNA/DNA NP treated Hbb^{th-4}/Hbb⁺ mice was harvested 3 days post NP delivery on E18.5 as previously reported (Coskun S et al., Cell Rep 9, 581-590 (2014)). Freshly isolated fetal bone marrow cells were suspended in ice-cold DMEM+ (Dulbecco's modified Eagle's medium, 10 mM HEPES, 2% fetal bovine serum [FBS]) at 10⁷ cells/ml and stained for 15 min on ice with anti-cKit-PE, anti-Scal-(PerCP)-Cy5.5, and FITC-conjugated lineage marker antibodies (CD4, CD8, Ter119, Gr-1, and CD45) (Thermo Scientific; Rockford, IL). Samples were then washed with 10× volume of HBSS+ (Hank's balanced salt solution, 10 mM HEPES, 2% FBS) and centrifuged at 2,000 rpm for 8 min at 4° C. Cell pellets were re-suspended in DMEM+ and samples were immediately sorted by flow cytometry (BD FACSAria).

### Deep Sequencing Analysis

gDNA from the bone marrow and liver of adult PNA/DNA NP treated Hbb^{th-4}/Hbb⁺ mice was harvested using the Wizard SV DNA Purification System (Promega, Madison, WI) according to manufacturer's instructions. gDNA was harvested from sorted fetal bone marrow and liver cells using a phenol-chloroform extraction method. Cells were digested overnight in10 mM Tris-HCl (pH 8), 150 mM NaCl, 20 mM ethylenediamine tetracetic acid and 1% sodium dodecyl sulfate, with proteinase K. Digests were subjected to extraction with phenol/chloroform/isoamyl alcohol followed by re-extraction with chloroform, precipitated with KOAc in EtOH, spun down and dried at room temperature and resuspended in dH₂O.

PCR reactions were performed with high fidelity TAQ polymerase (Invitrogen; Carlsbad, CA). Each PCR tube consisted of 28.2 μL dH₂O, 5 μL 10×HiFi Buffer, 3 μL 50 mM MgCl₂, 1 μL DNTP, 1 μL each of forward and reverse primer, 0.8 μL High Fidelity Platinum Taq Polymerase and 10 μL 40 ng/ml gDNA. Thermocycler conditions were as follows: 94° C. 2 min (94° C. 30 s, 55° C. 45 s, 68° C. 1 min)×35 cycles, 68° C. 1 min, hold at 4° C. PCR products were purified using the QIAquick PCR Purification Kit (Qiagen; Hilden, Germany). PCR products were prepared by end-repair and adapter ligation according to Illumina protocols (San Diego, CA), and samples were sequenced by the Illumina HiSeq 2500 with 75 paired-end reads at the Yale Center for Genome Analysis. Samples were analyzed as previously described. Briefly, paired-end reads were merged using PEAR (v. 0.9.6) (Zhang J et al., Bioinformatics 30, 614-620 (2014)). The merged reads were mapped to the loci of interest using BWA-MEM aligner (v. 0.7.12) (Li H et al., Bioinformatics 26, 589-595 (2010)). The nucleotide composition for each position in the alignment was obtained with in-house python scripts. To study off-target effects, the presence of 16 bp k-mers of the donor sequence was searched in the off-target sequencing libraries allowing for one mismatch using BBTools.

### Droplet Digital PCR

gDNA was extracted from all tissues using the Wizard SV DNA Purification System (Promega, Madison, WI) according to manufacturer's instructions. The concentration of extracted gDNA samples was measured using a QuBit® dsDNA BR assay kit (Invitrogen, Carlsbad, CA) according to manufacturer's instructions. Up to 80 ng of gDNA was used for each sample per reaction. PCR reactions were set up as followed: 11 μl 2×ddPCR™ supermix for probes (no dUTP) (Bio-Rad, Hercules, CA), 0.2 μl forward primer (100 μM), 0.2 μl reverse primer (100 μM), 0.053 μl β-thal probe (100 μM), 0.053 μl wild-type probe (100 μM) (Integrated DNA Technologies, Coralville, IA), 0.5 μl EcoR1, 10 μl gDNA and dH₂O. Droplets were generated using the Automated Droplet Generator (AutoDG™) (Bio-Rad). Thermocycling conditions were as follows: 95° C. 10 min, (94° C. 30 s, 55.3° C. 1 min—ramp 2° C./s)×40 cycles, 98° C. 10 min, hold at 4° C.

Droplets were allowed to rest at 4° C. for at least 30 minutes after cycling and were then read using the QX200™ Droplet Reader (Bio-Rad). Data were analysed using QuantaSoft™ software. Data are represented as the fractional abundance of the wild-type allele.

### Results

Every year, an estimated 8 million children are born worldwide with severe genetic disorders or birth defects. Of these diseases, hemoglobinopathies are the most commonly inherited single-gene disorders, with a global carrier frequency of over 5% (Modell B et al., Bull World Health Organ 86, 480-487 (2008)). Depending on the severity of the disease, children affected by β-thalassemia may require lifelong transfusions or bone marrow transplantation, which can lead to serious complications such as iron overload, sepsis, or graft-versus-host disease. Recent advances in non-invasive genetic testing allow for early gestation diagnosis of genetic disorders such as thalassemia (Fan H C et al., Nature 487, 320-324 (2012)), providing a window during which genetic correction could be achieved prior to birth.

In utero gene therapy thus far has focused on stem cell transplantation and viral-mediated gene delivery, reviewed in (Almeida-Porada G et al., Mol Ther Methods Clin Dev 5, 16020 (2016); McClain L E et al., Best Pract Res C/in Obstet Gynaecol 31, 88-98 (2016)), approaches that do not allow for correction of a gene in its endogenous environment. Site-specific gene editing to correct disease-causing mutations, however, can be coordinated efficiently and safely in postnatal animals via the intravenous or inhalational administration of polymeric, biodegradable nanoparticles loaded with peptide nucleic acids (PNAs) and single-stranded donor DNAs (McNeer N A et al., Gene Ther 20, 658-669 (2013); Bahal R et al., Nat Commun 7, 13304 (2016); McNeer N A et al., Nat Commun 6, 6952 (2015)). Here, the feasibility, safety, and efficacy of in utero gene editing mediated by PNA/DNA containing nanoparticles were determined.

Fetal surgeons and maternal fetal medicine physicians can safely access the amniotic cavity for amniocentesis and cannulate umbilical vessels for fetal blood transfusions under ultrasound guidance as early as 13 weeks of gestation in humans (Bang J, et al., Br Med J(Cin Res Ed) 284, 373-374 (1982); Kempe A et al., Ultrasound Obstet Gynecol 29, 226-228 (2007)). These procedures have been used in clinical practice since the 1980's and carry a very low risk of fetal loss (~1%) (Van Kamp I L et al., Am J Obstet Gynecol 192, 171-177 (2005)).

It was hypothesized that these same techniques could be safely used to introduce nanoparticles (NPs) in utero. This hypothesis was tested using poly(lactic-co-glycolic acid) PLGA NPs encapsulating fluorescent dyes. All NPs used were spherical visualized by Scanning electron microscope (SEM), and similar in size (~200 nm) and zeta potential (~−25 mV) measured by dynamic light scattering (DLS). Specifically, PLGA NPs encapsulating C6 have an average size of 205±71 nm, and zeta potential of −28.7±5 mV, and PLGA NPs encapsulating DiD have an average size of 214±92 nm, and zeta potential of −24.5±4 mV. Fluorescent NPs were administered to fetal B6 mice either intravenously via the vitelline vein or directly into the amniotic cavity at gestational ages later than E15: a glass micropipette injecting coumarin 6 (C6) NPs into the vitelline vein at E15.5; and intra-amniotic injection of C6 NPs at e16.5 (bottom) (n=69 fetuses IV; n=140 fetuses IA). The timing of these injections corresponds to a gestational time point in human development during which agents can be safely delivered via the umbilical vessels.

Administration of NPs to fetal mice results in particle retention within the fetuses with no detectable particle accumulation in the maternal mouse. Fetal brains, hearts, lungs, livers, guts, and kidneys were examined. Intra-vitelline vein delivery of fluorescent PLGA NPs results in widespread fetal particle distribution at both E15.5 and E16.5 with the most abundant NP accumulation in the fetal liver. Substantial accumulation of NPs in the fetal liver is expected during development because the extraembryonic vitelline veins anastomose to form the portal circulation.

During physiologic mammalian fetal development, the fetus breaths amniotic fluid into and out of the developing lungs, providing the necessary forces to direct lung development and growth (Kitterman J A, Clin Perinatol 23, 727-740 (1996)). Developing fetuses additionally swallow amniotic fluid, which aids the formation of the gastrointestinal tract (Mulvihill S. J. et al., J Surg Res 40, 291-296 (1986)). Thus, introduction of NPs into the amniotic fluid at gestational ages after the onset of fetal breathing and swallowing could result in direct delivery to the respiratory and gastrointestinal tracts, respectively. Intra-amniotic (IA) injection of fluorescent NPs at E15.5 did not lead to any detectable particle accumulation within the fetus. However, IA injection at E16.5, the expected time of onset of pronounced fetal breathing and swallowing (Douar A M et al., Gene Ther 4, 883-890 (1997)), resulted in particle accumulation in the fetal lung and gut. NP accumulation in the lung and gut was also observed after IA injections at E17.5 and E18.5, with increased intensity of accumulation at the later gestational ages.

In earlier work (McNeer N A et al., Mol Ther 19, 172-180 (2011); Schleifman E B et al., Mol Ther Nucleic Acids 2, e135 (2013)), it has been demonstrated the effectiveness of a gene editing approach that is mediated by tail-clamp peptide nucleic acid (tcPNA) oligomers and single-stranded “donor DNAs” encapsulated in NPs. tcPNAs are triplex-forming molecules that contain a specific sequence of nucleobases supported by a modified polyamide backbone. The backbone increases stability, confers resistance to intracellular degradation, and enhances binding affinity to complementary nucleic acids (Nielsen P E et al., Bioconjug Chem 5, 3-7 (1994)). Once inside a cell, the tcPNAs bind to a specific genomic target site via both Watson-Crick and Hoogsteen base-pairing (Egholm M et al., Nature 365, 566-568 (1993)). Formation of a PNA/DNA/PNA triplex induces endogenous DNA repair mechanisms that stimulate the recombination of the donor DNA molecule containing the correct sequence, resulting in specific, in situ gene correction (Rogers F A et al., Proc Natl Acad Sci USA 99, 16695-16700 (2002)). In previous work, the tcPNAs/DNAs were formulated in PLGA NPs for minimally invasive in vivo delivery in adult mice to create site-specific genomic modifications with extremely low off-target effects.

This work is believed to represent the first demonstration of NP delivery directly to a developing mammalian fetus for gene editing. Experiments were designed to evaluate the safety of this approach. No significant differences were observed in the survival of pups to weaning between those treated with NPs, either intravenously or intra-amniotically, compared to pups that received no in utero NP treatment were additionally observed (FIG. 11A). For the mice that lived to weaning, no significant differences in the long-term survival between untreated mice and those that received NP treatment in utero (FIG. 11B). After pups were born and matured, no significant differences were observed between untreated mice and those that received in utero NP treatment with respect to growth patterns or body weights (FIGS. 11C and 11D). No gross anatomical deformities, developmental abnormalities, or tumors were observed in the mice that received in utero NP treatments (n=72). Mice that had been treated with NPs in utero were able to have successful pregnancies and litters that were also free of gross abnormalities and tumors (n=14 litters, n=99 pups). Fetal plasma cytokine levels were measured 48 h after the IV delivery of PBS, blank NPs, and PNA/DNA NPs. There were no significant increases in levels of any of the cytokines measured in the NP treated groups compared to untreated fetuses (FIG. 11E), consistent with the preceding Examples, in which no significant cytokine elevations was observed after the IV delivery of NPs in post-natal animals.

During murine development, hematopoietic stem cells (HSCs) first emerge in the para-aortic splanchnopleure after the 7th day of gestation, E7.5, and later in the aorto-gonad-mesonephros, umbilical and vitelline vessels on E10.5 (Orkin S H, et al., Cell 132, 631-644 (2008); Dzierzak E et al., Nat Immunol 9, 129-136 (2008); de Bruijn M F et al., EMBOJ 19, 2465-2474 (2000)). HSCs that develop in these tissues do not differentiate, but go on to seed the fetal liver on E14.5, creating a niche that is capable of supporting HSC self-renewal. Within the fetal liver, HSCs undergo a massive expansion before further seeding the spleen, thymus, and finally the bone marrow at the beginning of E16.5 (Coskun S et al., Cell Rep 9, 581-590 (2014)). The rapid cycling and expansion of HSCs in the fetal liver is in stark contrast to adult bone marrow HSCs that are largely quiescent (Bowie M B et al., J Cin Invest 116, 2808-2816 (2006)). Notably, earlier work found elevated PNA/DNA mediated gene modification in hematopoietic stem and progenitors cells when compared to more differentiated cells (McNeer N A et al., Gene Ther 20, 658-669 (2013); Bahal R et al., Nat Commun 7, 13304 (2016)). The improved gene editing observed in stem and progenitor cells correlated with increased homology-dependent repair (HDR) activity. Importantly, HDR gene expression is elevated in S-phase, indicating that dividing cells may be more susceptible to PNA-mediated gene editing (Ciccia A et al., Mol Cell 40, 179-204 (2010); Chapman J R et al., Mol Cell 47, 497-510 (2012)). Thus, the ability to target or access this rapidly dividing stem cell population within the fetal liver may represent an important therapeutic opportunity. Given the observation that NPs accumulate in the fetal liver when administered at this time of rapid HSC expansion, it was a goal to determine if E15.5 IV injection of NPs encapsulating PNAs and donor DNAs targeting the β-globin locus could be used to correct the underlying genetic mutation and improve the disease phenotype in a transgenic mouse model of β-thalassemia. In this transgenic mouse model, the two (cis) murine β-globin genes were replaced with a single copy of the human β-globin gene containing a β-thalassemia-associated splice site mutation in intron 2 at position 654. No homozygous, Hbb^{th-4}/Hbb^{th-4}, mice survive postnatally. Heterozygous, Hbb^{th-4}/Hbb⁺, mice produce reduced amounts of mouse β-globin chains and no human β-globin, resulting in (3-thalassemia marked by microcytic anemia and splenomegaly (Lewis J et al., Blood 91, 2152-2156 (1998)).

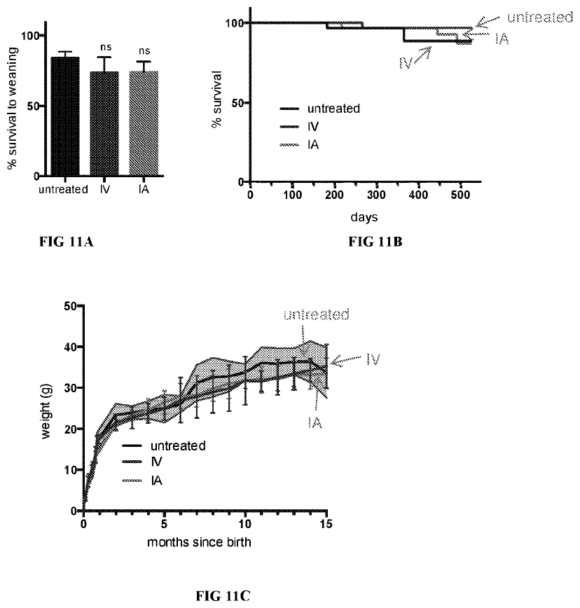

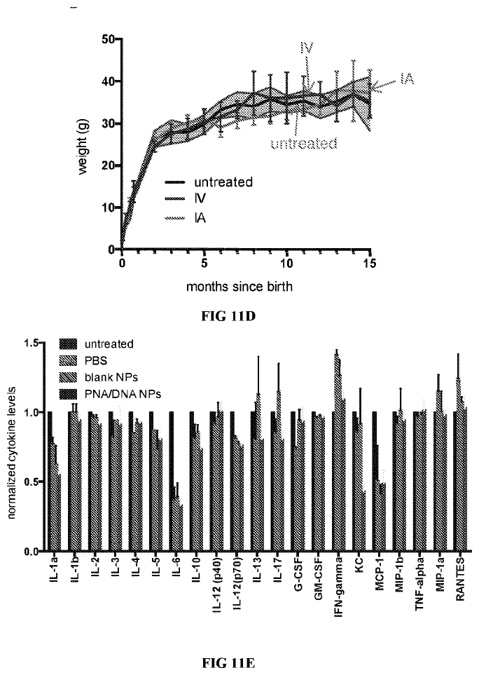

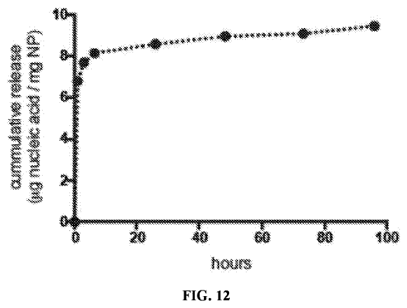

To examine in utero gene editing for postnatal disease improvement in these mice, a previously designed tcPNA/DNA pair, known to correct the underlying disease causing genetic mutation, was loaded into PLGA NPs. These NPs were also spherical, with sizes and zeta potentials similar to those used earlier with an average size of 258±83 nm, and zeta potential of −25.5±6 mV. Nucleic acid release profiles of the NPs in aqueous solution were also consistent with previous formulations (FIG. 12). The tcPNA used for this study was synthesized to contain mini-polyethylene-glycol group (miniPEG) modifications at the gamma position in the polyamide backbone (γPNA) of alternating residues in the Watson-Crick binding domain. These γPNAs pre-organize into a helical conformation that markedly increases DNA binding (Bahal R et al., Chembiochem 13, 56-60 (2012)). It has been previously shown that NP formulations containing γPNA and donor DNA produce gene editing at higher frequencies than unmodified PNAs both ex vivo and in vivo. It has also been shown that delivery of NPs containing this γtcPNA/DNA pair results in the correction of anemia in adult β-thalassemic mice prestimulated with stem cell factor.

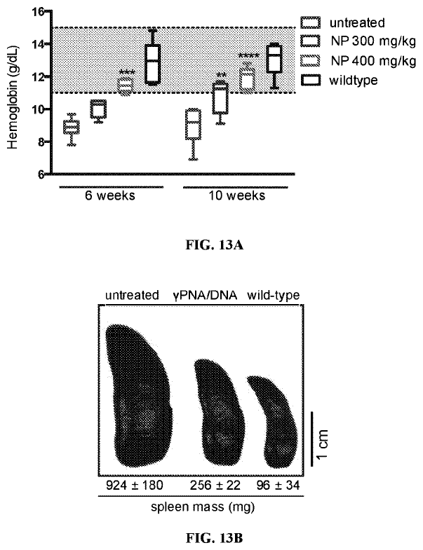

To test if in utero γPNA/DNA NP delivery could lead to postnatal disease improvement, NPs (300 or 400 mg/kg) were IV administered to each fetus via the vitelline vein at E15.5. The resulting pups were genotyped prior to weaning. The blood hemoglobin concentration of treated heterozygous mice was measured at six and ten weeks of age. At both doses, fetuses that were treated with NPs developed into adult mice with significantly higher levels of hemoglobin than untreated β-thalassemic mice. Notably, the higher dose of γPNA/DNA NPs resulted in greater elevation of hemoglobin concentrations into the wild-type range at both 6 and 10 weeks of age (FIG. 13A). The sustained elevation in postnatal hemoglobin was accompanied by a clear improvement in red blood cell (RBC) morphology on peripheral blood smear. Wright-Giemsa stained blood smears of γtcPNA/DNA NP treated mice 15 weeks post treatment were compared to untreated Hbb^{th-4}/Hbb⁺ and wildtype B6 mice. The peripheral smears of untreated mice display anisocytosis, poiklocytosis, and an abundance of target cells, all of which are hallmarks of β-thalassemia that are markedly reduced in the treated mice.

Hbb^{th-4}/Hbb⁺ mice have dramatically enlarged spleens, consistent with the splenomegaly seen in human patients. In utero NP administration produced a 73% reduction in splenic weight in treated mice, compared to controls, measured in adult animals at 30 weeks after the in utero NP treatment (FIG. 13B). H&E stained spleen sections from untreated Hbb^{th-4}/Hbb⁺ mice, Hbb^{th-4}/Hbb⁺ mice 10 weeks after E15.5 12 mg/ml NP treatment and wildtype mice were also compared. The observed reduction in splenomegaly correlated with improved splenic architecture in treated mice, with prominently defined red and white pulp. The demarcations between the white pulp and surrounding red pulp, seen in wild-type mice, were blurred in the untreated Hbb^{th-4}/Hbb⁺ mice as extramedullary hematopoiesis leads to expansion of the red pulp and disruption of the white pulp, resulting in splenomegaly. Decreased immunostaining in spleens for E-cadherin (immature erythroid precursors) and CD61 (megakaryocytes) in treated compared to untreated mice additionally suggests a reduction in extramedullary hematopoiesis. Taken together, the reduction of splenomegaly and improvement in splenic pattern in treated mice further indicates alleviation of anemia. In addition to elevated hemoglobin levels, improved RBC morphology, and reduction of splenomegaly, it was also observed that reticulocyte counts were significantly reduced in the peripheral blood of treated mice (FIG. 14A), indicating substantial correction of anemia.

Importantly, the mice treated with NPs in utero also showed a significant postnatal survival advantage compared to untreated controls. At 500 days after birth, in utero treated mice had 100% survival, in contrast to just 69% survival in the untreated group (FIG. 14B).

To establish the extent of gene editing that leads to these observed postnatal improvements in anemia, deep sequencing analysis was performed on genomic DNA extracted from total bone marrow and liver of mice 15 weeks post NP treatment. Correction of the targeted mutation was detected at a frequency of approximately 6% in both the total bone marrow (Table 8) and liver (Table 9). Deep sequencing was also used to assess off-target effects in the bone marrow and liver. Seven established off-target sites with partial homology to the binding site of the γPNA were also sequenced. The top seven gene loci in the mouse genome with partial homology to the 18 bp gPNA target site in b-globin intron 2 were identified in the preceding Examples, with the sequences as indicated. The size of the region sequenced around each site, the total number of amplicons sequenced and the number of amplicons with modified sequences are listed (Table 8 and Table 9). Extremely low frequencies of off-target editing in both the bone marrow and liver of treated mice were observed. The total measured off-target frequency is <0.000002%, 3,000,000-fold lower than the frequency of β-globin gene editing. In sorted E18.5 fetal bone marrow HSCs (Lin−, Sca1+, cKit+), 8.81% editing in bone marrow HSCs was observed by deep sequencing (Table 10). It is likely that the sustained correction of anemia and persistence of gene editing into the adulthood of treated mice is explained by the prevalence of a population of fetal HSCs, which were successfully edited by a single dose of γtcPNA/DNA loaded NPs.

TABLE 8
Deep sequencing analysis of targeted gene editing in the total
bone marrow

| | | Size of | | | |
|---|---|---|---|---|---|
| | Sequences of partial homology | region | Amplicons | Number | Frequency |
| Gene Locus | (5′ to 3′) | sequenced | sequenced | modified | (%) |
| β-globin | TGCCCTGAAAGAAAGA GA | 128 | 7230901 | 448040 | 6.20 |
| | (SEQ ID NO: 16) | | | | |
| Vascular cell | AGCCCTGAAAGAAAGA GA | 111 | 6280254 | 0 | 0 |
| adhesion protein | (SEQ ID NO: 196) | | | | |
| precursor 1 | | | | | |
| Polypyrimidine | GAACCTGAAAGAAAGA GA | 101 | 4452674 | 0 | 0 |
| tract binding | (SEQ ID NO: 197) | | | | |
| protein | | | | | |
| Protocadherin fat | CACCCTGAAAGAAAGA AG | 115 | 5742183 | 0 | 0 |
| 4 precursor | (SEQ ID NO: 221) | | | | |
| Olfactory | AAGCCTGAAAGAAAGA TT | 172 | 6148312 | 0 | 0 |
| receptor 266 | (SEQ ID NO: 222) | | | | |
| Syntaxin binding | AGAAATGAAAGAAAGA GA | 150 | 6660839 | 0 | 0 |
| protein | (SEQ ID NO: 200) | | | | |
| Muscleblind like | GGTGGTGAAAGAAAGA GA | 165 | 5553180 | 0 | 0 |
| protein | (SEQ ID NO: 201) | | | | |
| Ceruloplasmin | AGGACTGAAAGAAAGA GT | 154 | 6197021 | 0 | 0 |
| isoform | (SEQ ID NO: 202) | | | | |
| Total off-target | | | 41034463 | 0 | <0.000002 |

TABLE 9
Deep sequencing analysis of targeted gene editing versus off-
target effects in liver cells 15 weeks after a single in utero γtcPNA/DNA
NP treatment

| | | Size of | | | |
|---|---|---|---|---|---|
| | Sequences of partial homology | region | Amplicons | Number | Frequency |
| Gene Locus | (5′ to 3′) | sequenced | sequenced | modified | (%) |
| β-globin | TGCCCTGAAAGAAAGA GA | 128 | 6403869 | 463235 | 6.59 |
| | (SEQ ID NO: 16) | | | | |
| Vascular cell | AGCCCTGAAAGAAAGA | 111 | 7307127 | 0 | 0 |
| adhesion protein | GA (SEQ ID NO: 196) | | | | |
| precursor 1 | | | | | |
| Polypyrimidine | GAACCTGAAAGAAAGA | 101 | 4128040 | 0 | 0 |
| tract binding | GA (SEQ ID NO: 197) | | | | |
| protein | | | | | |
| Protocadherin | CACCCTGAAAGAAAGA AG | 115 | 6615009 | 0 | 0 |
| fat 4 precursor | (SEQ ID NO: 221) | | | | |
| Olfactory | AAGCCTGAAAGAAAGA TT | 172 | 6690832 | 0 | 0 |
| receptor 266 | (SEQ ID NO: 222) | | | | |
| Syntaxin binding | AGAAATGAAAGAAAGA | 150 | 6567607 | 0 | 0 |
| protein | GA (SEQ ID NO: 200) | | | | |
| Muscleblind like | GGTGGTGAAAGAAAGA | 165 | 6644517 | 0 | 0 |
| protein | GA (SEQ ID NO: 201) | | | | |
| Ceruloplasmin | AGGACTGAAAGAAAGA | 154 | 7784313 | 0 | 0 |
| isoform | GT (SEQ ID NO: 202) | | | | |
| Total off-target | | | 45717445 | 0 | <0.000002 |

TABLE 10
Deep sequencing analysis of E18.5 HSCs from the
bone marrow of γtcPNA/DNA NP treated fetal mice

| | Amplicons | Number | |
|---|---|---|---|
| Cell Type | sequenced | modified | Frequency (%) |
| Bone marrow | 4707133 | 414668 | 8.81 |
| HSCs | | | |

As another method of quantification, a droplet digital PCR (ddPCR) assay was developed and validated (FIG. 14D-14E). Probes differentiating the two alleles are specific for the gDNA template present in the reaction (beta-thal only and wild-type only controls). The method was used to quantify the percentage of modified β-globin gene alleles in 11 organs and tissues from the Hbb^{th-4}/Hbb⁺ mice that had been treated with NPs in utero (all samples were collected after 15 weeks, as above). In the bone marrow cells, ddPCR analysis confirmed an average of ~6% editing, consistent with the deep sequencing data (FIG. 14C). Gene editing was detected in several other tissues, but at lower frequencies than seen in the bone marrow (FIG. 14C).

Genetic correction during pregnancy could provide treatment or cure of numerous diseases and allow normal fetal development. Monogenic diseases that pose the risk of serious fetal, neonatal, and pediatric morbidity or mortality, such as β-thalassemia, are particularly attractive targets for in utero gene editing, reviewed in (Roybal J L et al., Semin Fetal Neonatal Med 15, 46-51 (2010)). β-thalassemia and other hemoglobinopathies are relatively common, may manifest early in life, and can be cured by low levels of functional protein activity. By delivering gene-editing therapies in utero, it is possible to gain access to dividing stem and progenitor cell populations, which can result in propagation of the corrected gene in all progeny cells. Here, it was demonstrated that in utero delivery of PNA/DNA nanoparticles is a safe and effective means of achieving clinically significant frequencies of site-specific gene editing that results in sustained postnatal alleviation of disease. This work establishes the framework for a clinically translatable approach to in utero gene editing that can be applied straightforwardly to numerous inherited disorders.

### Example 13: In Utero PNA/DNA Nanoparticle Treatment Yields Long-Term Gene Editing

### Materials and Methods

The fetuses of 654-eGFP mice were either untreated, treated with γPNA/DNA PLGA NPs either intravenously at E15.5 (15 μl of NPs resuspended at 9 mg/ml in 1×dPBS) or intra-amniotically at E16.5 (20 μl of NPs resuspended at 9 mg/ml in 1×dPBS), or blank PLGA NPs. Genomic DNA was extracted from the tissues (liver, lung, brain, gut, bone marrow, kidney, spleen, heart, pancreas, testes, seminal vesicle, ovary, and uterus) nine (9) months after in utero treatment. The presence of edited alleles was assayed by droplet digital PCR (ddPCR). The γPNA/DNA oligonucleotide are the same as those used in Example 12 above.

### Results

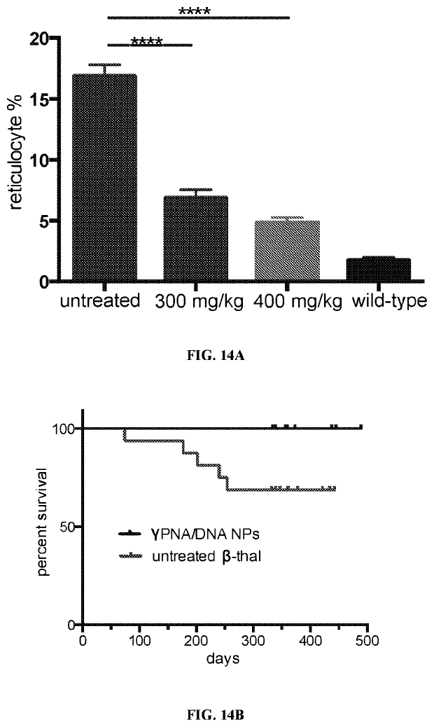

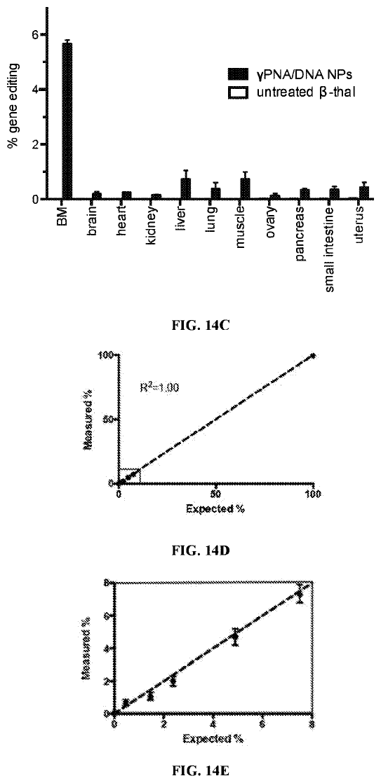

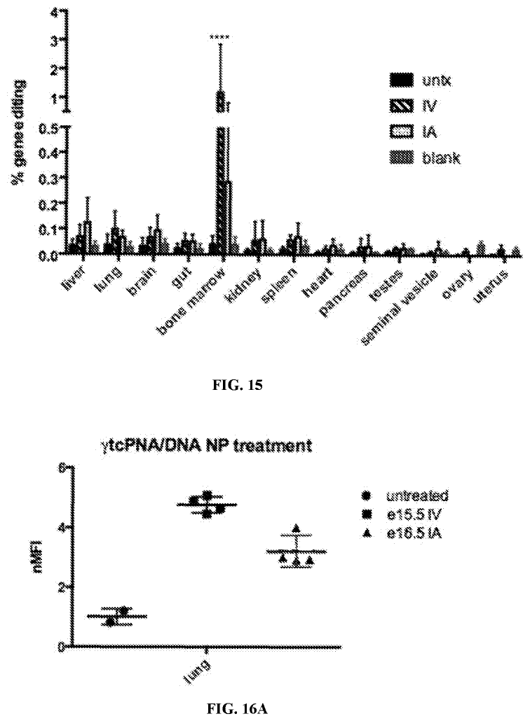

A significant level of gene editing was detected in the bone marrow of mice who received intravenous PNA/DNA NP treatment at E15.5; n=9 for all treatment groups; p<0.0001. See FIG. 15.

### Example 14: In Utero Gene Editing in the Lung after in Utero PNA/DNA Nanoparticle Treatment

### Materials and Methods

The fetuses of 654-eGFP mice were either untreated, or treated with γPNA/DNA PLGA NPs either intravenously at E15.5 (15 μl of NPs resuspended at 9 mg/ml in 1×dPBS) or intra-amniotically at E16.5 (20 μl of NPs resuspended at 9 mg/ml in 1×dPBS). Genomic DNA was extracted from the lung 4 days after in utero treatment. The γPNA/DNA oligonucleotide are the same as those used in Example 12 above.

### Results

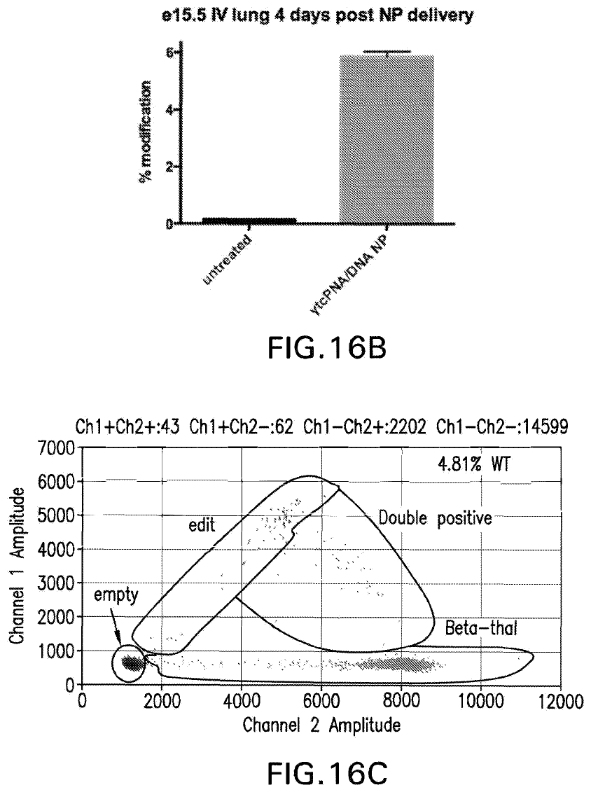

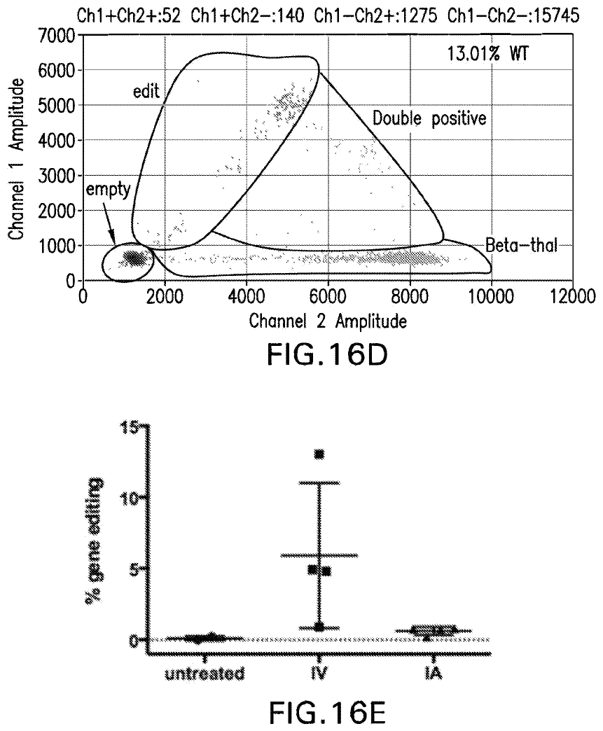

Gene editing was detected by flow cytometry (FIG. 16A), deep sequencing (intravenous treated fetuses only) (FIG. 16B), and ddPCR (FIGS. 16C, 16D, and 16E). In the 654-eGFP mouse model, the presence of green fluorescent cells, detected by flow cytometry, indicates gene editing; nMFI=normalized mean fluorescent intensity (FIG. 16A). FIGS. 16C and 16D) are representative 2D ddPCR plots of edited lungs 4 days post-intravenous in utero NP treatment. Looped dots labeled “empty” indicate empty droplets (no DNA template), looped dots labeled “beta-thal” indicate droplets loaded with unedited templates containing the beta-thalasemia 654-splice site mutation, looped dots labeled “edit” indicate droplets containing edited templates, and looped dots labeled “double positive” indicate droplets that contain both edited and un-edited templates. The aggregate, quantitative ddPCR data is shown in FIG. 16E. n=4 for all treatment groups.

### Example 15: In Utero Gene Editing in a F508Del Mouse Model of Cystic Fibrosis after E16.5 PNA/DNA Nanoparticle Delivery

### Materials and Methods

In Utero Gene Editing in a F508Del Mouse Model of Cystic Fibrosis after E16.5 PNA DNA NP Delivery

PLGA/PBAE/MPG NPs were loaded with (unmodified) PNA that binds in the murine CFTR gene and donor DNA. Zeta potential and Z-average were measured by DLS and the dry diameter derived from the electron micrograph using ImageJ.

| Zeta Potential | Z-Average | Diameter |
|---|---|---|
| (mv) | (DLS) (nm) | (nm) (dry) |
| 15.9 +/− 0.7 | 267.2 +/− 7.1 | 207 +/− 59.8 |

### Results

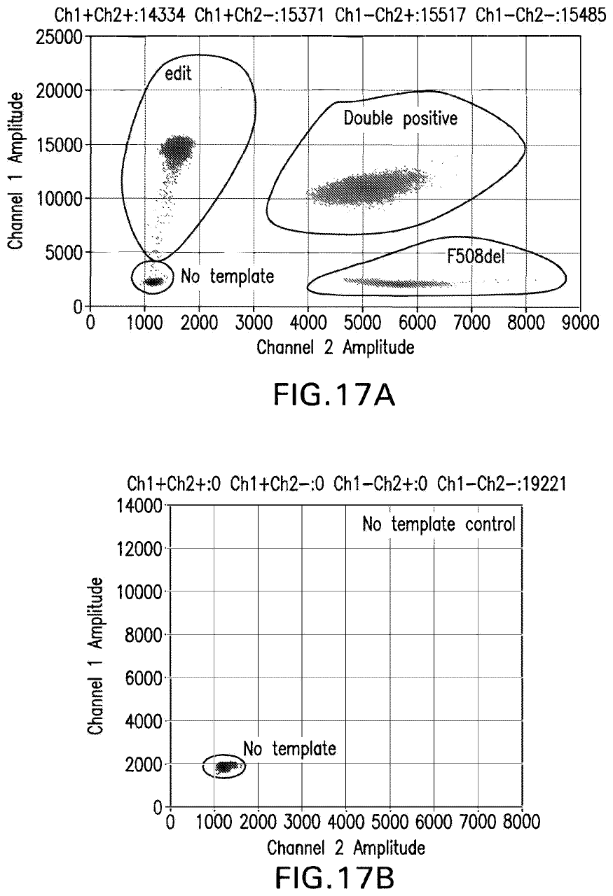

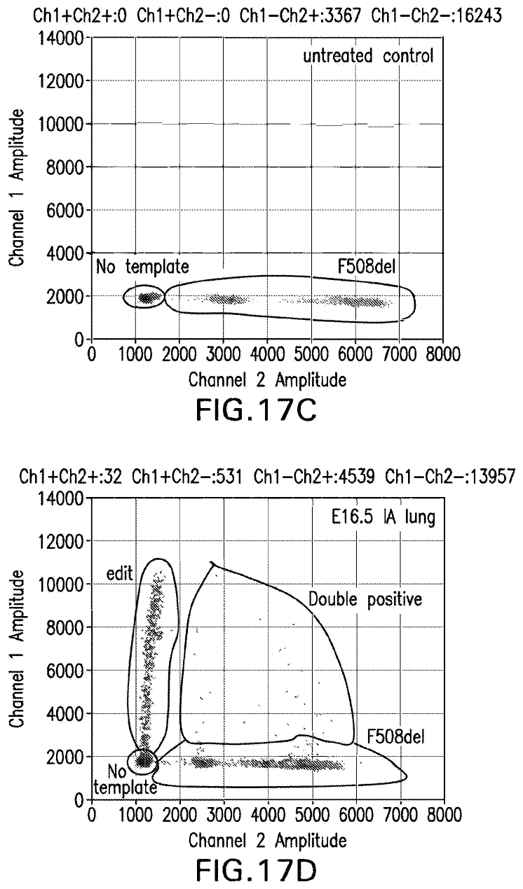

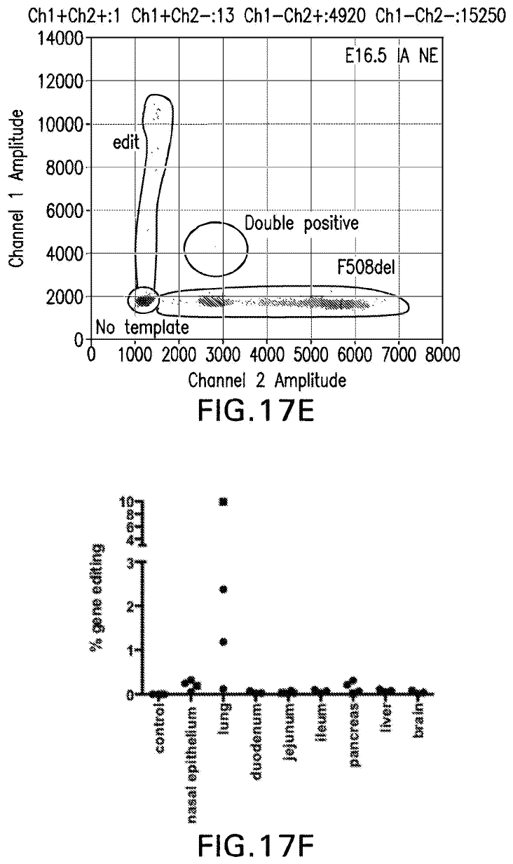

Droplet digital PCR was used to detect gene editing in the F508del model of cystic fibrosis. FIG. 17A illustrates the design of a ddPCR assay to detect murine CFTR gene editing. This 2D plot is the overlay of four samples (no template control (“no template”), ssDNA containing the F508del mutation (“F508del”), ssDNA containing the edited template (“edit”), and a sample containing both F508del and edited ssDNA (“double positive”)) to demonstrate the efficacy of the assay to discriminate the edited and unedited templates and to depict the expected location of dots containing each allele. FIG. 17B-17E are representative ddPCR 2D plots of no template control (17B), untreated gDNA (17C), gDNA extracted from the lung (17D) and nasal epithelium (17E) 8 months after E16.5 intra-amniotic treatment with PNA/DNA PLGA/PBAE/MPG NPs (20 μl of NPs resuspended to a concentration of 9 mg/ml in 1×dPBS). Looped dots labeled “no template” indicate empty droplets (no DNA template), looped dots labeled “F508del” indicate droplets containing the F508del mutation containing allele, looped dots labeled “edit” indicate droplets containing edited alleles, and looped dots labeled “double positive” indicate droplets that contain both edited and un-edited alleles.

FIG. 17F shows the results of ddPCR of genomic DNA from multiple CF tissues 8 months (square), 15 months (circle) after E16.5 intra-amniotic NP delivery, and control CF gDNA. Each fetus was given 20 ul of NPs resuspended to a concentration of 9 mg/ml in 1×dPBS, (n=4).

### Example 16: Characterizing the Biodistribution of Intravenously and Intra-Amniotically Injected Nanoparticles in Rat Fetuses

### Materials and Methods

Under isoflurane anesthesia, laparotomies were performed on time-dated pregnant Sprague-Dawley rats at varied time points from gestational age (E) 16-20 d. Using a micropipette attached to a pneumatic injector, rat fetuses were injected with poly(lactide-co-glycolide) (PLGA) nanoparticles (NPs) loaded with a green fluorescent dye (DiO) either directly into the amniotic cavity or IV through the vitelline vein. Three hours later the rat fetuses were sacrificed. The tissues were later examined using confocal microscopy to determine particle distribution.

### Results

The biodistribution of fluorescently-tagged nanoparticles was determined after being injected intravenously (IV) or intra-amniotically (IA) into rat fetuses. IV injections were performed successfully from days E16-18. The administered NPs were well distributed to lung, liver, and gastrointestinal tract, but were not found in skin or brain tissue. Successful IA injections were performed at age ranging from E16-E20. Particle distribution to the lung was not observed at early ages, but was observed at E19, suggesting that this is when the rat fetuses start to breathe and swallow. Characterizing the biodistribution of NPs in the rat is paramount prior to delivering NPs loaded with therapeutic agents to fetal rats. This study reveals that in rats, NP delivery throughout the fetus is most successful with IV injection; however NP delivery to lungs does occur at E19 and later via IA injection. Thus, it enables delivering active agent such as oligonucleotide to alter the development of the fetal lung in pulmonary disorders such as pulmonary hypoplasia.

### Example 17: In Utero Delivery of PNA/DNA Improves Impaired Response to Cyclic AMP Stimulation

### Materials and Methods

PLGA and PBAE/PLGA blended particles loaded with PNA were prepared as described herein, and in McNeer, et al., Nature Commun., 6:6952. doi: 10.1038/ncomms7952 (2015), and Fields, et al., Adv Healthc Mater., 4(3):361-6 (2015). doi: 10.1002/adhm.201400355 (2015) Epub 2014, administered to embryonic mice in utero: intravenously on E15.5 or intra-amniotic on E16.5, and the nasal potential difference measured.

The nasal potential difference is a non-invasive assay used to detect chloride transport in vivo. CF nasal epithelia in human and mice normally exhibit a large lumen-negative nasal potential that is amiloride-sensitive as well as a lack of activation of cyclic AMP-stimulated chloride efflux. This is in contrast to the response in wild-type or unaffected tissue, which exhibit a more modest amiloride-sensitive response and a robust cyclic AMP-stimulated chloride efflux. The lack of activation of cyclic AMP-stimulated chloride efflux in CF tissue is due directly to CFTR dysfunction and serves as a surrogate of CFTR activity.

### Results

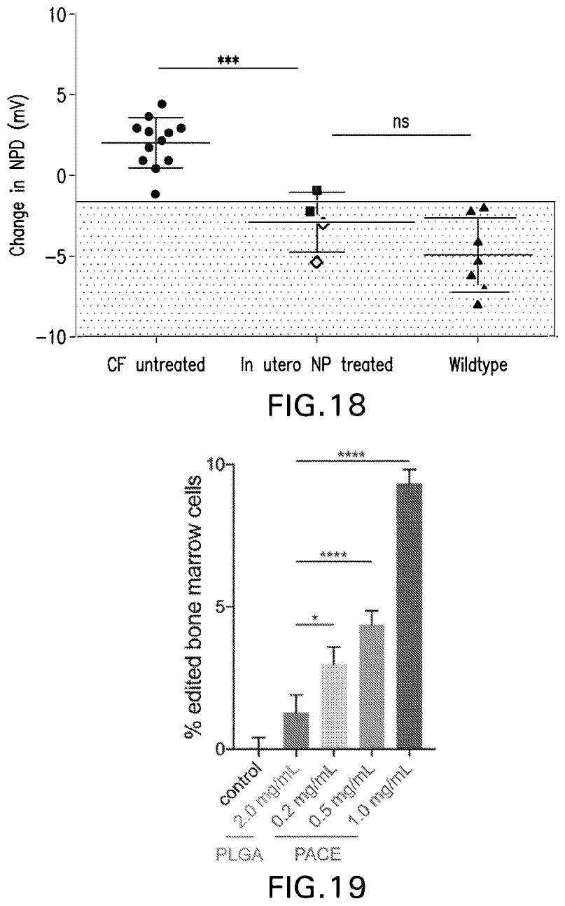

After in utero PNA/DNA NP treatment, the impaired response to cyclic AMP stimulation was partially corrected after intra-amniotic NP treatment and more robustly corrected after intravenous NP treatment. The treated mice hyperpolarize in response to forskolin, a stimulator of cyclic AMP, which is more characteristic of wild-type mice. The nasal potential difference (NPD) was measured four months after in utero NP treatment, indicating that a single, in utero NP treatment resulted in long term functional disease improvement. The results are presented in FIG. 18.

### Example 18: BM Cells Treated Ex Vivo with PNA/DNA-Loaded PACE NPs Exhibited Increased Gene Editing Compared to PLGA

Poly(amine-co-ester) (PACE) polymers are biocompatible/biodegradable, highly customizable, and mildly cationic, which aids in nucleic acid association. The polymers are synthesized in small batches via enzymatic copolymerization of diesters with amino-substituted diols. Classic PACE has three monomers: a lactone that confers hydrophobicity, an amino-diol that confers a cationic charge, and an ester.

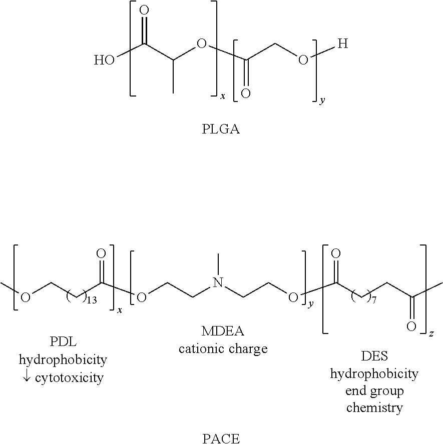

Altering feed monomer ratios, particularly the lactone, yields materials with different physical and chemical properties. We synthesized higher lactone content polymers (50-80 mol %), as these form solid NPs using the same emulsion methods as used for PLGA. Results shows that PACE nanoparticles are taken-up efficiently in primary bone marrow (BM) and mouse embryonic fibroblast (MEF) cells. Further, gene editing of the IVS2-654 mutation in BM cells treated ex vivo with PNA/DNA-loaded PLGA or 60% PDL PACE NPs showed increased gene editing by PACE compared to PLGA after 48 hours (FIG. 19).

### Example 19: Intra-Amniotic Delivery in a Rat Myelomeningocele Model to Determine the Effect of Particle Properties on Binding to MMC Defect

Myelomeningocele (MMC), or open spina bifida, is a devastating spinal birth defect that results in lifelong neurological morbidity. As the most severe form of spina bifida, MMC leads to lifelong lower body paralysis, bowel and bladder dysfunction. According to one study referenced by the Centers for Disease Control (CDC), the medical costs for MMC patients in the first year of life ranged from a median of $21,937 to a maximum of $1.35 million in 2012. The estimated annual cost of care for spina bifida patients in the United States in 1989 was $775 million, which would equal about $1.45 billion in today's dollars. The incidence of spina bifida is approximately 1 in 2000 to 2500 live births in the United States. This does not take into account the estimated 63% of MMC pregnancies in which the fetus is aborted, either spontaneously or intentionally. For mothers who have already given birth to one child with a neural tube defect, there is an approximately 4% risk that the next child will have spina bifida. For parents who have at least two children with neural tube defects, the risk of spina bifida in subsequent pregnancies increases to 10%.

The majority of spina bifida defects are found in the lower back, but defects can exist essentially anywhere along the spine, including the upper back and neck. Individual defects can vary significantly from patient to patient, and the term “spina bifida” encompasses a spectrum of open spinal defects. Patients with the mildest form of the anomaly, spina bifida occulta, have an open defect in the boney vertebral arches that normally protect the underlying spinal cord. Despite having this boney defect, these patients are usually neurologically normal, with no signs or symptoms except for a small dimple or tuft of hair overlying the defect. On the other end of the spectrum, patients with myelomeningocele (MMC), or open spina bifida, have no skin or soft tissue covering the boney defect along with posterior herniation of a developmentally abnormal spinal cord and meninges. Without any soft tissue or skin covering, the spinal cord is dangerously exposed to physical and chemical damage from the amniotic environment. Patients with MMC are usually born with paralysis of the lower body along with bowel and bladder dysfunction.

Prenatal diagnosis of MMC can be made as early as the first trimester using routine ultrasound surveillance. Since the advent of routine prenatal ultrasound, physicians have noticed that fetuses with MMC are able to move their legs as early as 16 weeks in gestation, but the patients' lower limb movements diminish gradually over the course of pregnancy. Based upon this observation, MMC experts have formulated a “two-hit” disease model in order to better explain the pathophysiology of MMC. The primary insult takes place during the 4th week of gestation when the caudal neuropore fails to close properly. As the fetus continues to grow, this defect in the caudal neural tube results in open exposure of the distal spinal cord. Secondary injury to the spinal cord occurs as it is exposed to intrauterine chemical and mechanical stresses over the course of gestation. The combined primary and secondary “hits” to the spinal cord result in lower extremity paralysis with bowel and bladder dysfunction by the time of birth in the majority of patients.

In addition, loss of hydrostatic pressure from leakage of cerebral spinal fluid (CSF) through the MMC defect results in herniation of the hindbrain (cerebellum) through the foramen magnum of the skull, a condition known as Chiari II malformation. The Chiari hindbrain malformation causes obstruction of CSF flow from brain to spinal column, and the CSF obstruction results in a condition known as hydrocephalus, in which there is rapid increase in head size and intracranial pressure from accumulated CSF. Even with standard post-natal closure of the defect, approximately 80% of MMC patients will require a permanent ventriculo-peritoneal (VP) shunt to drain the excess CSF from the brain into the abdominal cavity. Despite improved multi-disciplinary care, the neonatal mortality rate for MMC patients remains as high as 10%, and only approximately half of those who survive will be able to function independently as adults. As always, the best “treatment” for MMC is prevention, and folate diet supplementation at the turn of the 21st century appears to have decreased the prevalence of all neural tube defects by 21 to 35%. Still, despite large-scale folate supplementation, neural tube defects remain fairly common.

Current options for a pregnant woman faced with a prenatal diagnosis of MMC are termination, delivery and postnatal repair, or fetal surgery. Though prenatal surgery increases the number of patients with MMC who can walk, the results show that, even after the stress and enormous undertaking of prenatal surgery, approximately 60% of the patients still cannot walk. Another significant disadvantage to fetal surgery is a higher rate of premature birth and premature rupture of membranes. Though fetal surgery for MMC has resulted in significant medical advancements, it is expensive, somewhat inaccessible, resource intensive, and potentially dangerous for both mother and fetus.

The current standard of care is postnatal surgical reconstruction to cover the exposed spina elements and prevent infection and further spinal damage. Some centers have started performing prenatal surgery to cover the defect with adjacent skin and muscle, but this prenatal surgery is incredibly costly, very high risk for fetus and mother, highly restricted, and still leaves around 60% of patients unable to walk independently. Therefore, myelomeningocele remains a fairly common, devastating, and expensive problem for which there is no low-risk, inexpensive, effective, widely applicable treatment.

### Materials and Methods

Previous studies have established a reproducible MMC model in Sprague-Dawley rats. Pregnant Sprague-Dawley rats were fed all-trans retinoic acid, and this produces MMC defects in approximately 90% of the rat's offspring. Time-dated pregnant rats were gavage fed all-trans retinoic acid on day 10 of gestation to induce MMC. The control group received no further intervention. On day 18, groups 2, 3, 4 and 5 underwent laparotomy and intra-amniotic (IA) injections of various particles. Group 2 received poly(lactic-co-glycolic acid) (PLGA) nanoparticles (NPs) loaded with coumarin-6 (C6) fluorescent dye. Group 3 received alginate microparticles (MPs) loaded with bovine serum albumin (BSA) and Texas Red® dye. Group 4 received green fluorescent polystyrene MPs modified with terminal carboxyl groups (COOH), and Group 5 received unmodified green fluorescent polystyrene MPs.

All groups were sacrificed 3 hours after injections, and fetuses were examined under a fluorescence stereomicroscope. Images were analyzed with the Fiji image processing program. MMC defect-to-skin mean brightness ratios (MBR) were calculated using data from image overlays of equal area and analyzed by ANOVA with Tukey multiple comparisons test.

### Preparation of Particles

Alginate microparticles loaded with bFGF were resuspended in phosphate buffered saline and sonicated.

PRONOVA Sterile Alginate was purchased from NovaMatrix (Sandvika, Norway) and used in all experiments. Microparticles were fabricated. Briefly, alginate and hypromellose (Sigma-Aldrich) were dissolved in ultrapure water in a 10:1 (w/w) ratio to create a 1.25% alginate solution. Alginate was completely dissolved in an incubated orbital shaker under gentle agitation, at which time the protein cargo, either recombinant human bFGF (Creative Biomart, New York) stabilized with human serum albumin (HSA) or bovine serum albumin (BSA) conjugated to Texas Red® (BSA-TR) (ThermoFisher), was dissolved in the alginate solution. HSA has been shown to be well tolerated in rats and nude mice without evidence of allergy or anaphylaxis. Once combined, the alginate/protein solution was added dropwise into a homogenizing solution of iso-octane (Fischer) and 5% Span 80 (EMD Millipore) (v/v). Next, 30% Tween 80 (Sigma), 700 mM CaCl2) (Sigma) crosslinking solution, and isopropyl alcohol (AmericanBIO) were successively added to the homogenizing solution. After mixing, the solution was transferred to a new tube and washed twice in isopropyl alcohol. Following the final wash, the remaining alcohol was evaporated under a sterile air stream. The alginate particle pellet was then suspended in ultrapure water, aliquoted, flash frozen, and lyophilized for 24 hr until dry.

Goal protein concentrations were 1% TR-BSA (w/w alginate) and 1.5% bFGF (w/w alginate). All particles were stored at −20° C. until use. Particle size was measured with dynamic light scattering and surface charge (zeta potential) was measured in solution using a Malvern Zetasizer (Malvern Instruments, UK).

PLGA nanoparticles were loaded with the hydrophobic fluorescent dye, coumarin 6 (C6), to track the delivery and uptake of nanoparticles (NPs) in fetal tissues after intra-amniotic (IA) administration. PLGA (50:50 ester-terminated, 0.95-1.2 g/dl, LACTEL absorbable polymers; Birmingham, AL) NPs containing C6 (Sigma; St Louis, MO) were synthesized using a previously described single-emulsion solvent evaporation technique. C6 was added to the polymer solution at a 0.2% (w/w) dye-to-polymer ratio. Dynamic light scattering (DLS) was performed to measure the NP size (hydrodynamic diameter) and surface charge (zeta potential) using a Malvern Nano-ZS (Malvern Instruments, UK).

Green fluorescent polystyrene microparticles (MPs) with (PS-COOH) and without (PS-plain) carboxyl surface groups were purchased from Phosphorex (Hopkinton, MA). The average diameter for both types of PS MPs was 10 μm, and they had a concentration of 10 mg/mL in de-ionized water with a small amount of surfactant and 2 mM sodium azide. These polystyrene particles were used without any modification. Surface charge (zeta potential) was measured in solution using a Malvern Zetasizer (Malvern Instruments, UK).

### Injection and Binding Assay

Time-dated pregnant Sprague-Dawley dams were gavage fed ATRA at 60 mg/kg or 40 mg/kg on E10 to induce MMC, as described above. Control and ATRA-exposed dams between 14 and 21 days post-conception were anesthetized with inhaled isoflurane (3% v/v for induction, 2% v/v for maintenance). The gravid uterus was exposed through a midline laparotomy incision. For the binding specificity studies, lyophilized PLGA-C6 and Alg-BSA-TR were re-suspended by vortex and water bath sonication in 1× Dulbecco's phosphate-buffered saline (DPBS) to concentrations of 9 mg/mL (PLGA) and 18 mg/mL (Alg-BSA-TR). Polystyrene MPs (PS-COOH and PS-plain) were used at a concentration of 10 mg/mL. All particles were drawn up into a glass micropipette (tip diameter ~60 μm) and 20 μL injected directly into the amniotic cavity of each fetus using a pneumatic micro-injector (Narishige; Japan). Glass pipettes were pulled, cut, and ground. When injections were complete, the uterus was returned to the abdominal cavity, and the dam's abdominal wall fascia and skin were each closed with a running layer of 5-0 PDS II suture. During recovery, dams were placed in their cages over heating pads with easy access to water and chow, and they were monitored until they were awake and alert. For the PLGA, Alg-BSA-TR, and polystyrene studies, ATRA-exposed dams and wild type controls were sacrificed via chamber-inhaled carbon dioxide 3 hours after injections were completed. The uterus was exposed again, and individual fetuses were dissected free from the uterus within their amniotic sacks. Quality injection assurance was performed by observing the whole amniotic sac under a fluorescence microscope (Leica Microsystems, Wetzlar, Germany). If the amniotic fluid fluoresced under appropriate filter (e.g. green filter for C6), particles were considered to be successfully delivered into the amniotic space.

### Imaging and Measurements

Ex vivo fetal imaging of MMC defects was performed on a Leica M80 fluorescence stereomicroscope (Wetzlar, Germany). Images were analyzed using Fiji imaging software downloaded from the NIH website. Images from the fluorescence stereomicroscope were analyzed using the Fiji image processing program, and MMC defect-to-skin brightness ratios were calculated using raw integrated pixel densities from image overlays of equal area (400×400 pixel squares) (44). MMC defect-to-skin brightness ratios for all groups were compared using one-way analysis of variance (ANOVA) and Tukey multiple comparisons test.

### Amniotic Fluid Analysis

Amniotic fluid was obtained from fetal specimens ranging in age from E16.5 to E21. The amniotic fluid samples were centrifuged at 2000 rpm for 10 minutes to remove any blood clots. Rat/mouse bFGF levels were determined for untreated MMC specimens at age E16.5, E17.5, E18.5, and E21 using a Quantikine® ELISA kit (R&D Systems; Minneapolis, MN). Human bFGF concentration was measured in untreated MMC and Alg-HSA-bFGF treated MMC specimens at E21 using a Legend Max® Human FGF-basic ELISA Kit (BioLegend; San Diego, CA).

### Results

All dams (N=6) and fetuses (n=57) were viable at the time of sacrifice. PLGA NPs (p=0.0461), alginate MPs (p<0.0001), and polystyrene-COOH MPs (p<0.0001) had significantly higher defect-to-skin brightness ratios compared to controls.

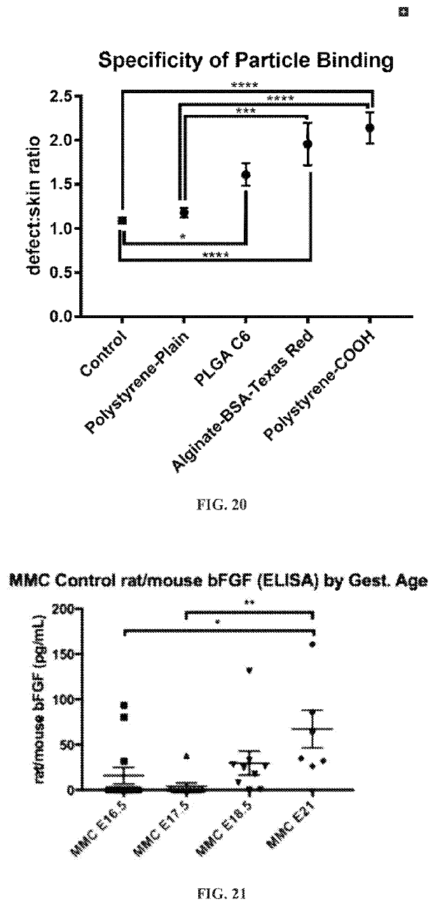

FIG. 20 shows specific and robust MMC defect binding was achieved by polystyrene-COOH MPs (MBR=2.139, 95% confidence interval, CI, 1.768-2.510), as well as alginate MPs (MBR=1.957, 95% CI 1.44-2.48) and PLGA NPs (MBR=1.61, 95% CI 1.34-1.88). Unmodified polystyrene MPs (PS-plain) had the weakest (p=0.9796) and least specific binding (MBR=1.18, 95% CI 1.07-1.30) compared to the non-injected controls (MBR=1.09, 95% CI 1.013-1.168).

Alginate particles were approximately 4.752+/−42 microns in diameter with a zeta potential of −35.3+/−2.5 mV. The PS-COOH particles were approximately 10 microns in diameter with a zeta potential of −47 mV. PS-plain particles were 10 microns in diameter with a zeta potential of −40 mV.

In short, intra-amniotic delivery of biodegradable alginate and PLGA particles to the MMC defect was feasible and did not cause immediate complications in rats. Polystyrene-COOH MPs, alginate MPs and PLGA NPs showed preferential binding to the MMC defect compared to surrounding skin. Specific binding to the MMC defect might be related to negative zeta potential or terminal functional groups. Preferential binding of biodegradable MPs and NPs to the MMC defect allows for targeted delivery of encapsulated therapeutic agents.

Open spina bifida, or MMC, is a devastating anomaly which affects approximately 4000 live births in the United States each year. Many affected neonates survive into adulthood and require full-time care in addition to multiple surgical procedures to correct hydrocephalus, bowel dysfunction, bladder dysfunction, decubitus ulcers, or other associated problems. Therefore, given that spina bifida is the most common neural tube defect, the condition can be very expensive at both personal and societal levels. Though it has shown promising results, fetal surgery is maximally invasive, highly exclusive (excludes 80% of patients screened), expensive, and fraught with risk for both mother and fetus. Attempts at less invasive laparoscopic and fetoscopic interventions have yet to yield results as promising as open fetal surgery, and they involve increased physiologic stress. Furthermore, it seems that even fetal MMC repair is performed too late to adequately preserve exposed neural tissue.

Recent studies in rats and sheep have shown that a gelatin sponge scaffold impregnated with bFGF can encourage ingrowth of skin with partial or even complete coverage of the spinal cord with new skin in some cases. However, in order to place a gelatin scaffold over an MMC defect, one would have to perform fetoscopy or open fetal surgery, and thus the treatment would still be invasive. Experiments were designed to determine whether it would be possible to achieve similar results in a less invasive way through the injection of biodegradable particles carrying therapeutic growth factors. Nano- and microparticles can be engineered to target specific tissue either by surface modification or antibody conjugation. Experiments were designed to investigate whether any particles could be appropriate vehicles for the controlled delivery of growth factors to the MMC defect. bFGF was chosen as the therapeutic growth factor. This is believed to be the first study involving the intra-amniotic injection of biodegradable particles for the delivery of growth factors to the MMC defect.

Given a high rate of spontaneous abortions and very severe defects, it was initially believed that the dose being administered was too high. Upon further investigation, it was discovered that there was a difference in the way in which the ATRA-olive oil was prepared. The investigators in a previous study measured out the amount of ATRA according to each individual dam's weight and suspended that amount of ATRA in 2 mL of olive oil. Due to the logistics of the gavage process, the exact dose of ATRA for each individual dam was not easy to determine before adding it to the olive oil. In order to streamline the process, the total weight of ATRA per vial was measured and suspended in a volume of olive oil required to create a stock solution of 12 mg/mL ATRA in olive oil. The volume administered per dose was then altered according to the dam's weight to achieve the appropriate total ATRA dose. When the dose was decreased to 40 mg/kg, isolated MMC yields around 90% were immediately achieved, and therefore, this dose was used for the remainder of the experiments. The exact reason or reasons for the difference in caudal regression and exencephaly rates was not identified, but it might have been related to the nutritional makeup of the chow. Nevertheless, the isolated MMC specimens resembled those of prior studies on gross examination and histology.

A determination of which biodegradable particles were best for delivering therapeutic agents to the fetal MMC defect was made. Initial experiments included the intra-amniotic injection of PLGA particles encapsulating coumarin-6 green fluorescent dye. Interestingly, without any modifications, the PLGA particles appeared to bind preferentially to the MMC defect of E21 fetuses. E21 is almost full term, and so therapeutic injections were preformed earlier in gestation in order to allow time for tissue growth. Therefore, intra-amniotic injections of PLGA-C6 were performed in fetuses ranging in age from E14 to E18. After qualitative analysis under the fluorescence stereomicroscope, it appeared that the PLGA-C6 particles bound most specifically to the MMC defect at E18 and slightly less specifically at E17. This MMC binding specificity was not nearly as pronounced at age E16 and younger perhaps due to the inherent qualities of the more immature epidermis. E18 was selected as the proper time point to test preferential particle binding.

Alginate microparticles, were also included in the study. PLGA-C6 NPs were compared to Alg-BSA-TR MPs in E18 fetuses and both bound specifically to the MMC defect. Alginate MPs bound slightly more specifically to the MMC defect compared to PLGA NPs.

To investigate why particles preferentially bind to the MMC defect, polystyrene MPs of known size with and without surface carboxyl groups were tested. Given that the particles with more negative zeta potential (PS-COOH vs PS-plain) bound more specifically to the defect, it is possible that either terminal COOH groups, negative zeta potential, or both characteristics lead to improved binding specifically to the MMC defect. Alginate MPs are rich in COOH groups and have a negative zeta potential, and therefore, it is possible that these characteristics of alginate particles contribute to better MMC defect binding compared to PLGA.

### Example 19: Intra-Amniotic Delivery of Fibroblast Growth Factor-Loaded Alginate Microparticles

### Materials and Methods

### Animal Preparation

At a specific time point, pregnant rats are fed all-trans retinoic acid, and this produces MMC defects in approximately 90% of the rat's offspring. Sodium alginate microparticles are produced in the Biomedical Engineering lab and loaded with human basic fibroblast growth factor (bFGF) with a small amount of human serum albumin (HSA) added for stability. At a specific time point, the pregnant rats are placed under general anesthesia, and their uterus is exposed via midline laparotomy.

Each MMC dam was gavage fed either 60 mg/kg or 40 mg/kg of 97% pure all-trans retinoic acid (ATRA) (Across Organics; Morris Plains, NJ) dissolved in extra virgin olive oil (Whole Foods Market; Austin, TX) between 8 PM to 9 PM on gestation day E10. All wildtype dams were left alone until sacrifice. In prior studies, this method has induced MMC in fetal rats at an acceptably high rate (>80%). Early on during the study, however, high rate of caudal regression, exencephaly, and severely large MMC defects were noticed. The dose was decreased from 60 mg/kg to 40 mg/kg and achieved a 97% MMC rate at the new dose. 40 mg/kg was used for the remainder of the study.

### Administration

Using a pulled glass pipette with approximately 60 micron tip, 30 microliters of suspended microparticles are injected into the amniotic space surrounding the rat fetuses. The uterus is then returned to its normal position within the abdomen, and the pregnant dam's abdominal wall is closed. In a human, this procedure would be performed in a manner similar to amniocentesis, during which a ultrasound-guided needle is inserted into the amniotic sac to withdraw a small amount of amniotic fluid for genetic testing.

Specifically, time-dated pregnant Sprague-Dawley dams at E17 and E18 were placed under general anesthesia with inhaled isoflurane (Henry Schein) (3% vol/vol for induction, 2% vol/vol for maintenance). The gravid uterus was exposed through a midline laparotomy incision. Lyophilized Alg-HSA-BFGF was re-suspended in sterile Dulbecco's PBS to a concentration of 20 mg/mL by gentle vortex and water bath sonication. The particle solution was drawn up into a glass micropipette (tip diameter ~60 μm). A pneumatic micro-injector (Narishige; Japan) was used to inject a volume of L into the amniotic cavity of each fetus via a ventral approach. Great care was taken to avoid inadvertently injecting into vessels, placenta, or the fetus itself, and placement of the needle tip was indirectly confirmed by a small retrograde flash of amniotic fluid into the tip immediately prior to injection. When injections were complete, the uterus was returned to the abdominal cavity, and the dam's abdominal wall fascia and skin were each closed with a running layer of 5-0 PDS II suture. During recovery, dams were placed in their cages over heating pads with easy access to water and chow, and they were monitored until they were awake and alert.

### Histological Analysis

Control and injected ATRA-exposed dams were sacrificed via chamber-inhaled carbon dioxide prior to term on E21. The gravid uterus was exposed via laparotomy and removed. Great care was taken to remove each individual fetus from the uterus with its amniotic sac intact. Each fetus's amniotic sac was then removed. Ex vivo fetal imaging of MMC defects was performed on a Leica M80 fluorescence stereomicroscope (Wetzlar, Germany). Fetuses were fixed for 2 days in 4% (w/v) paraformaldehyde (Electron Microscopy Sciences; Hartfield, PA) in PBS and transferred at room temperature in 70% (v/v) ethanol in distilled water to Yale Pathology Tissue Service (YPTS) for paraffin embedding and sectioning. Cut sections were stained with hematoxylin-eosin, pancytokeratin AE1/AE3, or trichrome per YPTS protocols. Sections were obtained at 50 □m intervals beginning at the superior aspect of the MMC defect and moving caudally down through the defect. The slides were imaged on a Zeiss Axio Scope (Carl Zeiss Microscopy; Germany).

### Magnetic Resonance Imaging (MRI)

Wild type and control MMC fetuses were fixed in 4% PFA (w/v) in PBS for 2 weeks and delivered to the Yale Magnetic Resonance Research Center (MRRC). Each specimen was placed into a custom-built MRI-compatible tube filled with Fluorinert—an MRI susceptibility-matching fluid (Sigma-Aldrich). MR images were obtained on an 11.7 T horizontal bore scanner (Bruker, Billerica, MA) with a bore size of 21 cm and maximum gradient strength of 400 mT/m, using a custom-made 1H radio frequency volume coil (4 cm diameter). The DTI experiments were performed using an echo-planar imaging (EPI) spin-echo diffusion-weighted sequence with 4 shots, a diffusion gradient of 6 ms and a delay between the two diffusion gradients of 12 ms. 30 contiguous slices of 0.5 mm thickness were acquired at a resolution of 192×128 with a field of view of 34 mm×22 mm using a repetition time (TR) of 8 s, an echo time (TE) of 27 ms and 32 averages. 35 different images were acquired for each slice, 30 corresponding to various non-collinear diffusion gradient directions with b=1,000 s mm-2 and 5 with no diffusion gradients. Fractional anisotropy (FA) and average diffusion coefficient (ADC) images were generated from the EPI-based DTI images in BioImage Suite.

### Statistical Analysis

Rat/mouse bFGF concentration data along the gestational timeline were obtained via ELISA and analyzed using ordinary one-way analysis of variance with Tukey multiple comparisons test. Adjusted P value<0.05 was considered significant. Post-injection rat/mouse and human bFGF concentration data obtained at E21 via ELISA was analyzed with a 2-tailed unpaired t-test with Welch's correction because equal standard deviations could not be assumed between groups. Adjusted P value less than 0.05 was considered significant. Defect-to-skin brightness ratio data for the fluorescence binding study was analyzed using ANOVA with Tukey multiple comparisons test. Again, an adjusted P value<0.05 was considered significant. Statistical analyses were performed using GraphPad Prism (version 7; GraphPad Software; La Jolla, CA).

### Results

Alginate microparticles have been studied to deliver proteins and growth factors to specific tissues, as well as in topical delivery of drugs to chronic wounds. However, they have not been used for the controlled release delivery of growth factors to a specific area of the fetus in utero, especially the open spinal defect of a fetus with myelomeningocele.

### Fetal Delivery of Alg-HSA-bFGF

Before injecting exogenous bFGF into the amniotic space, the baseline levels of detectable bFGF in rat MMC amniotic fluid was measured. To determine what the baseline bFGF level is in rat amniotic fluid at various time points around the time of the injection, rat/mouse bFGF levels for untreated MMC specimens at age E16.5, E17.5, E18.5, and E21 were obtained and as shown in FIG. 21. The level of bFGF in rat amniotic fluid increases toward full term to a mean of 67 pg/mL (range 26 to 161 pg/mL). After analyzing the data using ordinary ANOVA with Tukey multiple comparisons, the differences between E16.5 (15.8 pg/mL), E17.5 (4.1 pg/mL) and E21 (67.3 pg/mL) were found to be statistically significant (E16.5 vs E21, P=0.02; E17.5 vs E21 P=0.006).

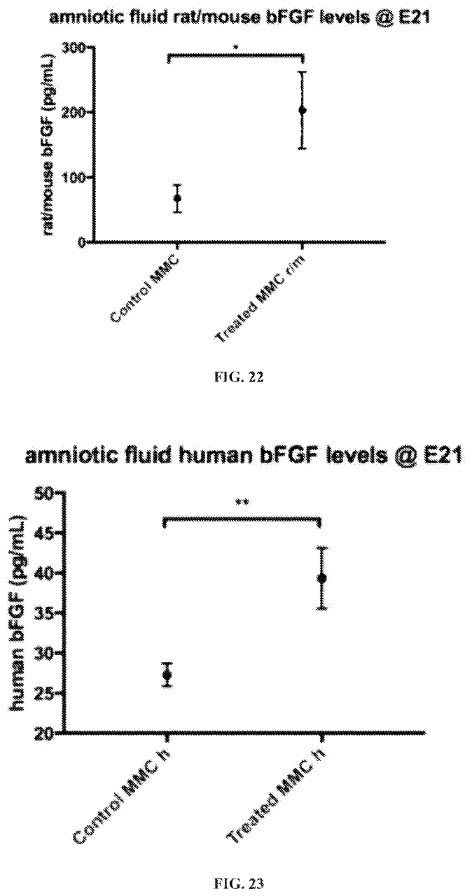

For the Alg-HSA-bFGF study, treated MMCs had higher amniotic fluid levels of both human (39.3+/−3.8 pg/mL vs 27.3+/−1.4 pg/mL, p=0.0046) and rat/mouse bFGF (202.9+/−59 pg/mL vs 67.3+/−20.9 pg/mL, p=0.041) compared to control MMCs (FIGS. 22 and 23). Obtaining amniotic fluid via needle aspiration is difficult toward the end of gestation due to the increasing fetus-to-amniotic fluid ratio. By E21, the amniotic fluid is so low that there is no visible target for needle aspiration, and the fluid must be obtained by opening the amniotic sac and allowing the small amount that remains to drain out. Despite the challenge in obtaining enough amniotic fluid per specimen, values of both rat/mouse and human bFGF were obtained in control MMC and injected MMC specimens. The control MMC bFGF amniotic fluid concentrations ranged from 0 to 161 pg/mL, and the levels appeared to increase significantly toward full term.

Next, the amount of rat/mouse and human bFGF were measured in control and injected MMC fetuses. The levels of both human and rat/mouse bFGF were significantly higher in the injected amniotic fluid vs the control fluid at day E21. While the human bFGF level was approximately 12 ng/mL higher in the injected specimens compared to control, the rat/mouse bFGF level was approximately 135 ng/mL higher in the injected amniotic fluid by E21 (p=0.04). The reason for the larger increase in rat/mouse bFGF over human bFGF is not clear, but may be related to a host inflammatory response to the injections. The user manual of the rat/mouse Quantikine® ELISA kit states that its detection antibody cross-reacts with human recombinant bFGF, but the extent of cross-reactivity is not made clear in the user manual provided by the company. Also, according to the user manual included in the LEGEND Max™ human bFGF ELISA kit, it should not detect rat/mouse bFGF. Therefore, the fact that the control human bFGF level is higher than zero indicates an high background, and this high background could be due to user error or a faulty kit.

All dams that underwent laparotomy and IA injections with Alginate-HSA-bFGF, blank Alginate, Alginate-BSA-TR, bFGF alone and PBS were alive at the time of sacrifice (N=22). 150 of 239 fetuses (62.8%) that received IA injections at E17.5 were viable at E21. Fetal loss between the time of injection and sacrifice was not significantly higher in any group that received injections. Un-injected specimens (0 of 24, 0%), those injected with PBS (0 of 13, 0%) and those injected with bFGF alone (0 of 42, 0%) showed no coverage of the defect. Of the remaining control groups, 2 of 18 (11%) treated with Alginate-BSA-TR and 3 of 19 (16%) treated with Alginate-Blank appeared to have at least partial coverage of the MMC defect. Fluorescent stereomicroscopy of the MMC specimen treated with Alginate-BSA-TR revealed positive red fluorescence throughout a layer of tissue covering the defect with negligible fluorescent signal from surrounding normal skin. This finding suggests that the alginate particles aggregated specifically over the MMC defect and likely accomplished cellular or paracellular delivery of fluorescently tagged BSA. On histological analysis of the same specimen, the red fluorescent layer of tissue over the MMC defect had the appearance of abnormal, hyperkeratotic and partially formed skin.

Gross and histomorphological differences were also disclovered between the control MMCs and several of the treated specimens. Even on gross analysis, 7 out of 34 (20.6%) of the injected specimens observed appeared noticeably smaller with opaque rinds overlying the exposed nerve tissue. Although smaller defects might be the result of natural variation among the MMC defects; the analyses of a large number of untreated fetal rat MMC specimens have not revealed any similar results on either gross or histological analysis.

Following injection of particles loaded with growth factor, Alginate-HAS-bFGF, 18 of 61 (30%) fetuses treated with Alginate-HSA-bFGF had evidence of soft tissue coverage that was significant compared to un-injected (P=0.0021), PBS (P=0.0297), and bFGF controls (P<0.0001). Histological analysis of specimens treated with Alginate-HSA-bFGF that were positive for tissue coverage on gross inspection also showed complete skin covering over the spinal cord.

Slides were stained with trichrome and a pan cytokeratin (AE1/AE3) immunohistochemical stain. Trichrome is excellent for visualizing normal skin as well as collagen in the dermis and subdermal tissue. The immunohistochemistry AE1/AE3 stain is a cocktail of antibodies against cytokeratin found in epithelial cells. In the skin, AE1/AE3 specifically stains the epidermis, eccrine glands, and folliculosebaceous-apocrine unit. The AE1/AE3 stain was used in a previously published study which used bFGF-impregnated gelatin sponges to grow new skin-like tissue over the defect.

In control MMC slide images, the 2 flattened and exposed hemicords were identified, adjacent thin epithelia layer, and the normal appearing skin located far from the nerve tissue. None of the control slides stained strongly for AE1/AE3, especially not within the epithelial tissues surrounding the cord. For the treated specimens, it was immediately apparent that there was abnormal-appearing skin covering the spinal cord more inferiorly within the defect, as compared to the control. Additionally, one of the specimens appeared to have a thick film overlying both the new skin and the spinal cord. The film appeared to be composed of scattered red blood cells, some nucleated cells, and spherical empty structures consistent with alginate particles. The overall structure seemed benign (it washed away during the preparation of pan cytokeratin AE1/AE3 slides). Moreover, the extremely positive staining of pan cytokeratin AE1/AE3 directly over the spinal cord indicates that those areas of skin are relatively new or at least producing excess keratins compared to the normal skin at the periphery of the slides. In sum, with trichrome staining and analysis at the same level within the MMC defect, the treated MMCs appeared to have a layer of abnormal skin covering the spinal cord, whereas the control spinal cord was flattened and completely exposed. In addition, treated specimens stained strongly for pan cytokeratin AE1/AE3 specifically in the area of the skin covering the spinal cord while the control did not have significantly increased staining in the area of the defect or the skin immediately surrounding it. This skin is notably different than the native skin that has lower levels of pan cytokeratin staining and the absence of hair follicles. Moreover, treated specimens did not have significant staining within the skin distant from the MMC defect.

Put together, the pan cytokeratin staining pattern suggests that the skin seen overlying the spinal cord in these treated specimens is new, growing rapidly, over-producing keratins, or all of the above.

### Fetal MRI for the Microstructural Analysis Offetal MIC

Micro MRI scans were completed for 4 wild type and 4 control MMC specimens, from which Diffusion Tensor Imaging (DTI) images were produced. With DTI imaging, software can be used to assign colors (red, green, or blue) to identify the direction of a water diffusion vector in plane x, y, or z. There were differences in the passive diffusion of water throughout the spinal cord and the brain. The spinal cord of the MMC specimen was almost entirely red, which indicated a significant passive diffusion of water in the cranio-caudal direction. Meanwhile, the wild type specimen had a markedly thinner stripe of red in the center of its cord, perhaps identifying the passive diffusion of water along the central canal. Altogether, there are marked differences between wild type and untreated MMC specimens on DTI images.

### CONCLUSION

Intra-amniotic injection of biocompatible PLGA and alginate particles is feasible and safe in the immediate peri-injection period. Both PLGA and alginate bind preferentially to the MMC defect compared to the fetal skin, and this preferential binding is best at gestation day E17.5 to E18. Negative zeta potential and/or surface carboxyl groups might be factors that improve binding to the MMC defect. Although PLGA and alginate particles bind to the MMC defect without any surface modifications, future attempts at improved MP targeting with surface group modifications or antibody conjugation might allow for more optimal delivery of bFGF to the MMC defect. Intra-amniotic injection of Alg-HSA-bFGF microparticles at gestational ages E17 to E18 leads to the growth of new skin over the MMC defect in the ATRA-induced rat MMC model.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method comprising administering in utero to an embryo or fetus, an effective amount of a composition comprising a therapeutic, prophylactic or diagnostic agent encapsulated, entrapped, complexed to or dispersed in particles to treat a disease or condition in the embryo or fetus.

2. The method of claim 1, wherein the particles are selected from the group consisting of microparticles, nanoparticles, and mixtures thereof.

3. The method of claim 2, wherein the particles are formed of a polymer, copolymer or polymer blend selected from the group consisting of poly(lactic acid) (PLA), poly(glycolic acid) (PGA), poly(lactide-co-glycolide) (PLGA), polyesters, polyanhydrides, poly(ortho)esters, poly(butic acid), poly(valeric acid), poly(caprolactone), poly(hydroxyalkanoates), poly(lactide-co-caprolactone), poly(beta-amino) esters (PBAEs) and poly(amine-co-ester) polymers (PACE).

4. The method of claim 2, wherein the particles comprises polymer or co-polymer selected from the group consisting of alginate, chitosan, and poly(HEMA).

5. The method of claim 1, wherein the particles comprise nanoparticles and the agent is a gene editing composition that corrects a mutation in the genome of the fetus or embryo.

6. The method of claim 5, wherein the nanoparticles comprise PLGA, a blend of PLGA and (PBAE), or PACE.

7. The method of claim 5, wherein the gene editing composition comprises a peptide nucleic acid (PNA) tail clamp that enhances recombination of a donor oligonucleotide in the fetal or embryonic genome to correct the mutation relative to the donor oligonucleotide alone.

8. The method of claim 7, wherein the gene editing composition further comprises the donor oligonucleotide.

9. The method of claim 7, wherein the PNA tail clamp comprises at least one PNA oligomer.

10. The method of claim 9, wherein the at least one PNA oligomer of the PNA tail clamp is a modified PNA oligomer comprising at least one modification at a gamma position of a backbone carbon.

11.-13. (canceled)

14. The method of claim 1, wherein the particles comprise microparticles and the active agent is a growth factor that treats a structural defect in the embryo or fetus.

15. The method of claim 14, wherein the microparticles comprise alginate.

16. The method of claim 14, wherein the growth factor is a basic fibroblast growth factor.

17. The method of claim 14, wherein the structural defect leads to muscular dystrophy, Type 2 diabetes, or spina bifida.

18. The method of claim 1, wherein the composition is administered to a fetus.

19. The method of claim 1, wherein the composition is administered by injection or infusion into the vitelline vein.

20. The method of claim 1, wherein the composition is administered by intraamniotic sac injection.

21. A composition for use in the method of claim 1 comprising a therapeutic, prophylactic or diagnostic agent encapsulated, entrapped, complexed to or dispersed in particles to treat a disease or condition in the embryo or fetus and a pharmaceutically-acceptable carrier, wherein the composition is either formulated for infusion or injection into a vitelline artery or a vitelline vein or formulated for intraamniotic sac injection.

22. (canceled)

23. The composition of claim 1, wherein the therapeutic, prophylactic or diagnostic agent is an anti-infectious agent.

24. The composition of claim 23, wherein the anti-infectious agent is an antibacterial agent.
